(19) United States
(12) Patent Application Publication
(10) Pub. No.: US 2009/0306392 A1
(43) Pub. Date: Dec. 10, 2009

(54) GSM INTERMEDIATES

(76) Inventor: Chih Yung HO, Lansdale, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/253,298

(22) Filed: Oct. 17, 2008

Related U.S. Application Data
(60) Provisional application No. 60/981,257, filed on Oct. 19, 2007.

(51) Int. Cl. C07D 211/34; C07C 229/00

(52) U.S. Cl. 546/239; 560/48

(57) ABSTRACT

The present invention relates the use of compounds having the general Formula I, wherein the definitions or R¹ and R² are provided in the specification. Said compounds of Formula I are useful for the synthesis of a variety of γ-secretase modulators, which are in turn useful for the treatment of diseases associated with γ-secretase activity, including Alzheimer's disease.

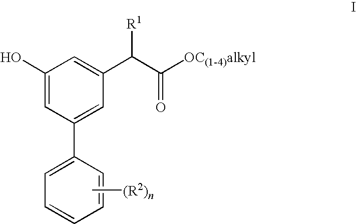

### CROSS REFERENCE TO RELATED APPLICATIONS

This application claims priority of the benefits of the filing of U.S. Provisional Application Ser. No. 60/981,257, filed Oct. 19, 2007. The complete disclosures of the aforementioned related U.S. patent application is/are hereby incorporated herein by reference for all purposes.

The present invention relates the use of compounds having the general Formula I, wherein the definitions or R¹ and R² are provided in the specification. Said compounds of Formula I are useful for the synthesis of a variety of γ-secretase modulators, which are in turn useful for the treatment of diseases associated with γ-secretase activity, including Alzheimer's disease.

### BACKGROUND OF THE INVENTION

Alzheimer's Disease (AD) is a progressive neurodegenerative disorder marked by loss of memory, cognition, and behavioral stability. AD afflicts 6-10% of the population over age 65 and up to 50% over age 85. It is the leading cause of dementia and the third leading cause of death after cardiovascular disease and cancer. There is currently no effective treatment for AD. The total net cost related to AD in the U.S. exceeds $100 billion annually.

AD does not have a simple etiology, however, it has been associated with certain risk factors including (1) age, (2) family history (3) and head trauma; other factors include environmental toxins and low level of education. Specific neuropathological lesions in the limbic and cerebral cortices include intracellular neurofibrillary tangles consisting of hyperphosphorylated tau protein and the extracellular deposition of fibrillar aggregates of amyloid beta peptides (amyloid plaques). The major component of amyloid plaques are the amyloid beta (A-beta, Abeta or Aβ) peptides of various lengths. A variant thereof, which is the Aβ1-42-peptide (Abeta-42), is believed to be the major causative agent for amyloid formation. Another variant is the Aβ1-40-peptide (Abeta-40). Amyloid beta is the proteolytic product of a precursor protein, beta amyloid precursor protein (beta-APP or APP).

Familial, early onset autosomal dominant forms of AD have been linked to missense mutations in the β-amyloid precursor protein (β-APP or APP) and in the presenilin proteins 1 and 2. In some patients, late onset forms of AD have been correlated with a specific allele of the apolipoprotein E (ApoE) gene, and, more recently, the finding of a mutation in alpha2-macroglobulin, which may be linked to at least 30% of the AD population. Despite this heterogeneity, all forms of AD exhibit similar pathological findings. Genetic analysis has provided the best clues for a logical therapeutic approach to AD. All mutations, found to date, affect the quantitative or qualitative production of the amyloidogenic peptides known as Abeta-peptides (Aβ), specifically Aβ42, and have given strong support to the “amyloid cascade hypothesis” of AD (Tanzi and Bertram, 2005, Cell 120, 545). The likely link between Aβ peptide generation and AD pathology emphasizes the need for a better understanding of the mechanisms of Aβ production and strongly warrants a therapeutic approach at modulating Aβ levels.

The release of Aβ peptides is modulated by at least two proteolytic activities referred to as β- and γ-secretase cleaving at the N-terminus (Met-Asp bond) and the C-terminus (residues 37-42) of the Aβ peptide, respectively. In the secretory pathway, there is evidence that β-secretase cleaves first, leading to the secretion of s-APPβ (sβ) and the retention of a 11 kDa membrane-bound carboxy terminal fragment (CTF). The latter is believed to give rise to Aβ peptides following cleavage by γ-secretase. The amount of the longer isoform, Aβ42, is selectively increased in patients carrying certain mutations in a particular protein (presenilin), and these mutations have been correlated with early-onset familial Alzheimer's disease. Therefore, Aβ42 is believed by many researchers to be the main culprit of the pathogenesis of Alzheimer's disease.

It has now become clear that the γ-secretase activity cannot be ascribed to a single particular protein, but is in fact associated with an assembly of different proteins. The gamma-secretase activity resides within a multiprotein complex containing at least four components: the presenilin (PS) heterodimer, nicastrin, aph-1 and pen-2. The PS heterodimer consists of the amino- and carboxyterminal PS fragments generated by endoproteolysis of the precursor protein. The two aspartates of the catalytic site are at the interface of this heterodimer. It has recently been suggested that nicastrin serves as a gamma-secretase-substrate receptor. The functions of the other members of gamma-secretase are unknown, but they are all required for activity (Steiner, 2004. Curr. Alzheimer Research 1(3): 175-181).

Thus, although the molecular mechanism of the second cleavage-step has remained elusive until present, the γ-secretase-complex has become one of the prime targets in the search for compounds for the treatment of Alzheimer's disease.

Various strategies have been proposed for targeting gamma-secretase in Alzheimer's disease, ranging from targeting the catalytic site directly, developing substrate-specific inhibitors and modulators of gamma-secretase activity (Marjaux et al., 2004. Drug Discovery Today Therapeutic Strategies, Volume 1, 1-6). Accordingly, a variety of compounds were described that have secretases as targets (Lamer, 2004. Secretases as therapeutics targets in Alzheimer's disease: patents 2000-2004. Expert Opin. Ther. Patients 14, 1403-1420.)

Indeed, this finding was recently supported by biochemical studies in which an effect of certain NSAIDs on γ-secretase was shown (Weggen et al (2001) Nature 414, 6860, 212 and WO 01/78721 and US 2002/0128319; Morihara et al (2002) J. Neurochem. 83, 1009; Eriksen (2003) J. Clin. Invest. 112, 440). Potential limitations for the use of NSAIDs to prevent or treat AD are their inhibition activity of Cox enzymes, which can lead to unwanted side effects, and their low CNS penetration (Peretto et al., 2005, J. Med. Chem. 48, 5705-5720).

Thus, there is a strong need for novel compounds which modulate γ-secretase activity thereby opening new avenues for the treatment of Alzheimer's disease.

The object of the present invention is to provide such compounds.

### SUMMARY OF THE INVENTION

The invention relates to the use of compounds of Formula I

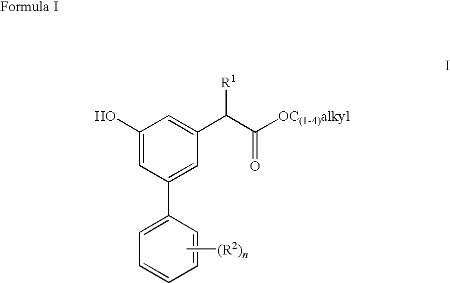

wherein:
R¹ is C₍₁₋₄₎alkyl, or C₍₁₋₅₎alkenyl;
R² is selected from the group consisting of —H, —C₍₁₋₄₎alkyl, —OC₍₁₋₄₎alkyl, —NO₂, —CN, —NH₂, —F, —Br, —Cl, and —CF₃; and
n is an integer from 1-3
to make γ-secretase modulators of Formulas XIII-XXVII

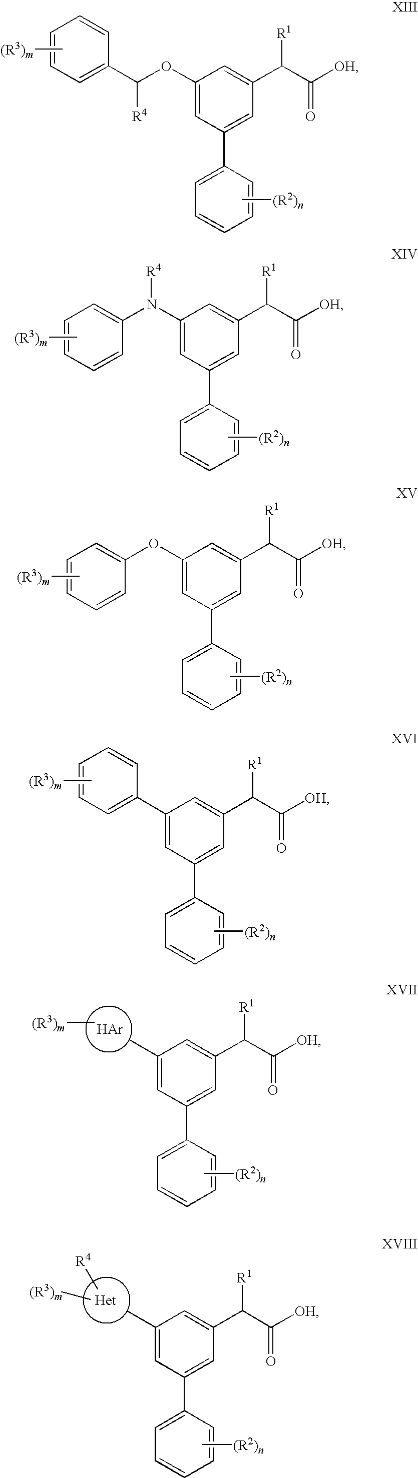

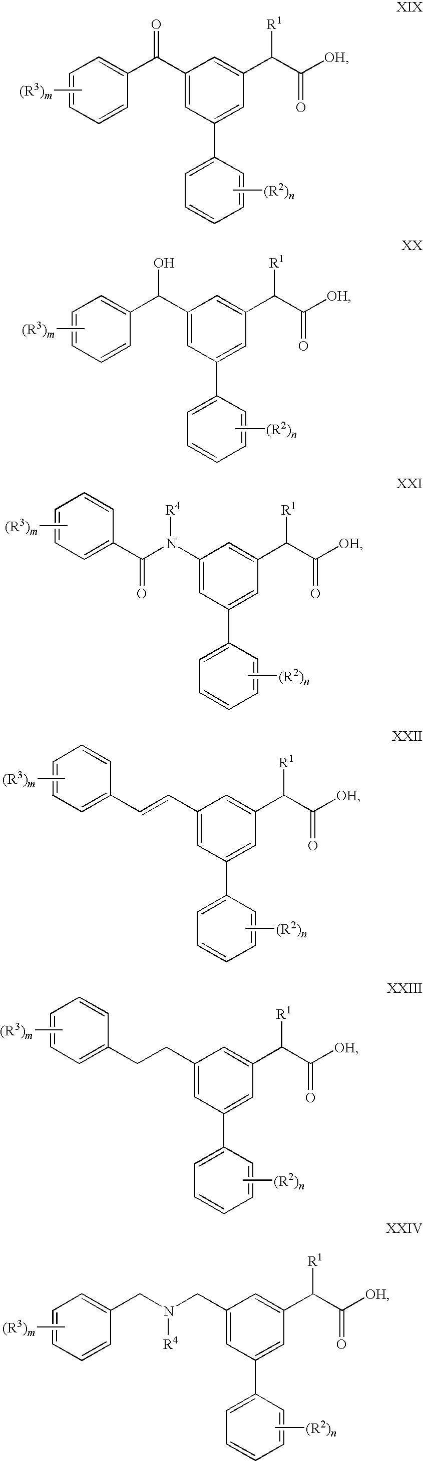

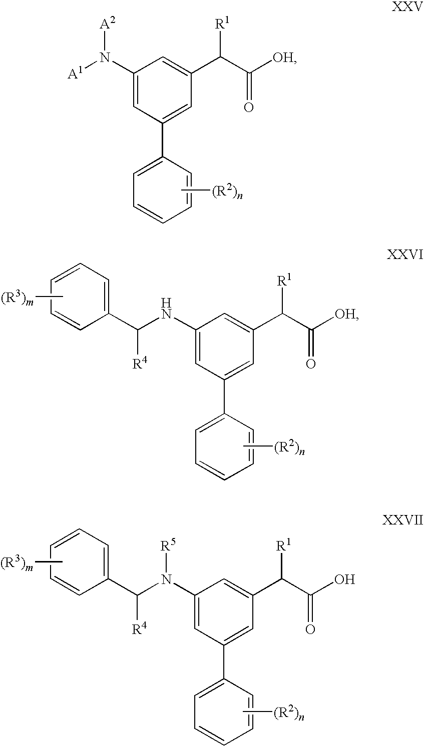

wherein
Het is heterocyclyl;
HAr is heteroaryl;
R³ is selected from the group consisting of —H, —C₍₁₋₄₎alkyl, —OC₍₁₋₄₎alkyl, —NO₂, —CN, —NH₂, —F, —Br, —Cl, and —CF₃;
R⁴ is C₍₁₋₄₎alkyl;
R⁵ is C₍₁₋₄₎alkyl;
A¹ is H or —C₍₁₋₄₎alkyl;
A² is —C₍₁₋₄₎alkyl;
alternatively, A¹ and A² may be taken together to form a nitrogen containing heterocyclic ring selected from the following:

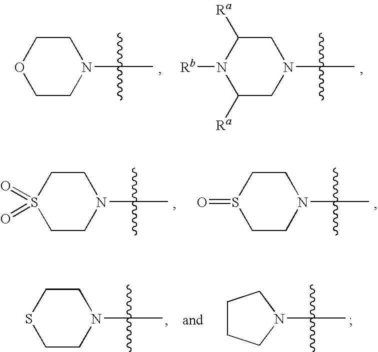

wherein:
R^{a} is H, CH₃, or CH₂CH₃; and
R^{b} is H, or CH₃;
   m is an integer from 1-3;
   n is an integer from 1-3; and
   solvates, hydrates, prodrugs, and pharmaceutically acceptable salts thereof.

### DETAILED DESCRIPTION OF THE INVENTION

The invention relates to the use of compounds of Formula I

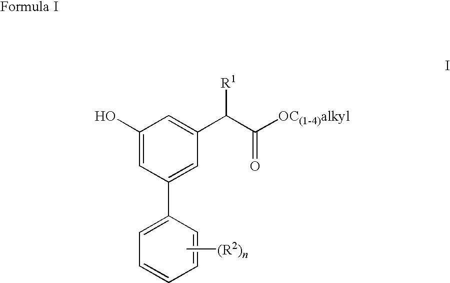

wherein:
R¹ is C₍₁₋₄₎alkyl, or C₍₁₋₅₎alkenyl;
R² is selected from the group consisting of —H, —C₍₁₋₄₎alkyl, —OC₍₁₋₄₎alkyl, —NO₂, —CN, —NH₂, —F, —Br, —Cl, and —CF₃; and
n is an integer from 1-3
to make γ-secretase modulators of Formulas XIII-XXVII

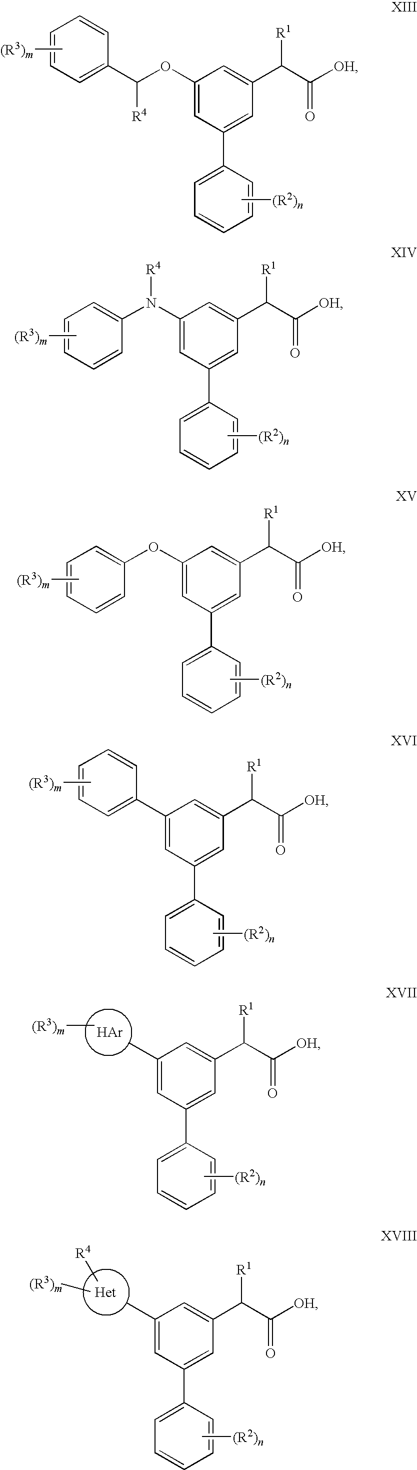

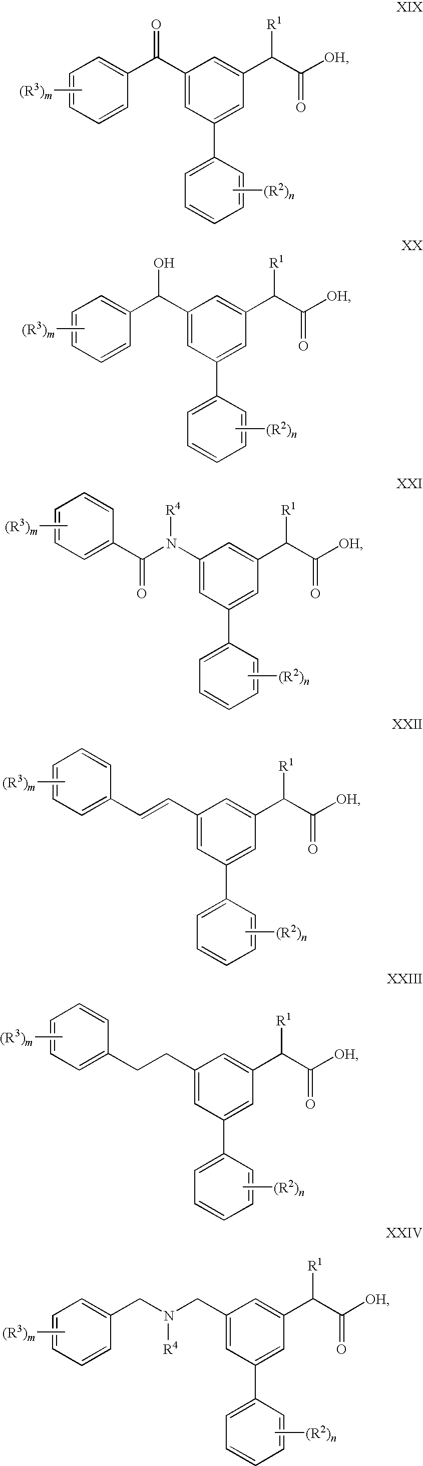

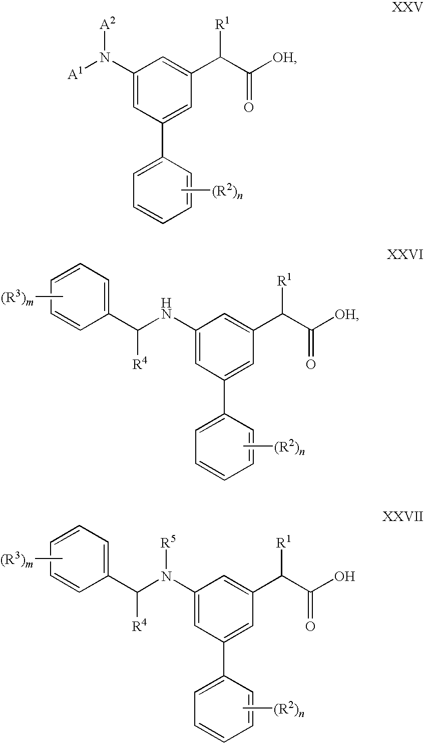

wherein
Het is heterocyclyl;
HAr is heteroaryl;
R³ is selected from the group consisting of —H, —C₍₁₋₄₎alkyl, —OC₍₁₋₄₎alkyl, —NO₂, —CN, —NH₂, —F, —Br, —Cl, and —CF₃;
R⁴ is C₍₁₋₄₎alkyl;
R⁵ is C₍₁₋₄₎alkyl;
A¹ is H or —C₍₁₋₄₎alkyl;
A² is —C₍₁₋₄₎alkyl;
alternatively, A¹ and A² may be taken together to form a nitrogen containing heterocyclic ring selected from the following:

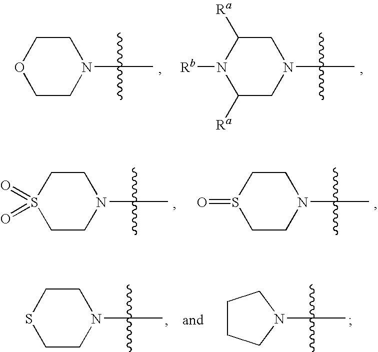

wherein:
R^{a} is H, CH₃, or CH₂CH₃; and
R^{b} is H, or CH₃;
   m is an integer from 1-3;
   n is an integer from 1-3; and
   solvates, hydrates, prodrugs, and pharmaceutically acceptable salts thereof.

In another embodiment of the invention

The invention relates to the use of compounds of Formula I

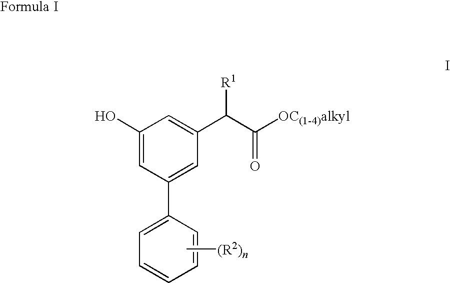

wherein:
R¹ is C₍₁₋₄₎alkyl, or C₍₁₋₅₎alkenyl;
R² is selected from the group consisting of —H, —C₍₁₋₄₎alkyl, —OC₍₁₋₄₎alkyl, —NO₂, —CN, —NH₂, —F, —Br, —Cl, and —CF₃; and
n is an integer from 1-3
to make γ-secretase modulators of Formula XIII

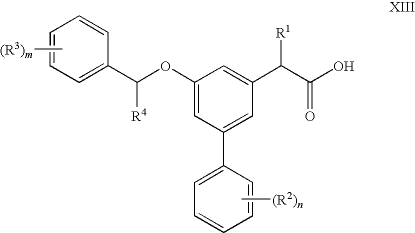

wherein
m is an integer from 1-3;
R³ is selected from the group consisting of —H, —C₍₁₋₄₎alkyl, —OC₍₁₋₄₎alkyl, —NO₂, —CN, —NH₂, —F, —Br, —Cl, and —CF₃;
R⁴ is C₍₁₋₄₎alkyl; and
solvates, hydrates, prodrugs, and pharmaceutically acceptable salts thereof.

In another embodiment of the invention

The invention relates to the use of compounds of Formula I

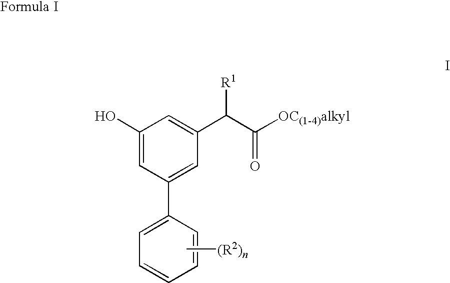

wherein:
R¹ is C₍₁₋₄₎alkyl, or C₍₁₋₅₎alkenyl;
R² is selected from the group consisting of —H, —C₍₁₋₄₎alkyl, —OC₍₁₋₄₎alkyl, —NO₂, —CN, —NH₂, —F, —Br, —Cl, and —CF₃; and
n is an integer from 1-3
to make γ-secretase modulators of Formula XIV

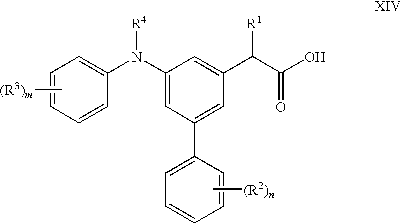

wherein
m is an integer from 1-3;
R³ is selected from the group consisting of —H, —C₍₁₋₄₎alkyl, —OC₍₁₋₄₎alkyl, —NO₂, —CN, —NH₂, —F, —Br, —Cl, and —CF₃;
R⁴ is C₍₁₋₄₎alkyl; and
solvates, hydrates, prodrugs, and pharmaceutically acceptable salts thereof.

In another embodiment of the invention

The invention relates to the use of compounds of Formula I

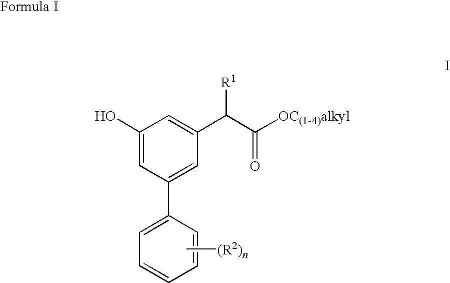

wherein:
R¹ is C₍₁₋₄₎alkyl, or C₍₁₋₅₎alkenyl;
R² is selected from the group consisting of —H, —C₍₁₋₄₎alkyl, —OC₍₁₋₄₎alkyl, —NO₂, —CN, —NH₂, —F, —Br, —Cl, and —CF₃; and
n is an integer from 1-3
to make γ-secretase modulators of Formula XV

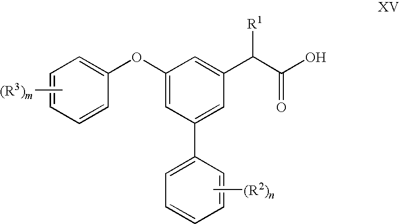

wherein
m is an integer from 1-3;
R³ is selected from the group consisting of —H, —C₍₁₋₄₎alkyl, —OC₍₁₋₄₎alkyl, —NO₂, —CN, —NH₂, —F, —Br, —Cl, and —CF₃; and
solvates, hydrates, prodrugs, and pharmaceutically acceptable salts thereof.

In another embodiment of the invention

The invention relates to the use of compounds of Formula I

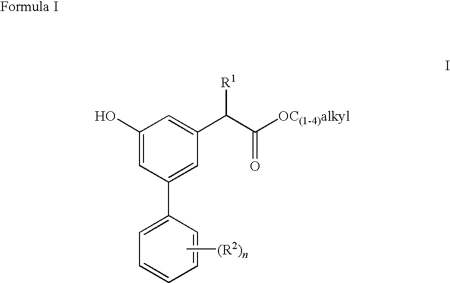

wherein:
R¹ is C₍₁₋₄₎alkyl, or C₍₁₋₅₎alkenyl;
R² is selected from the group consisting of —H, —C₍₁₋₄₎alkyl, —OC₍₁₋₄₎alkyl, —NO₂, —CN, —NH₂, —F, —Br, —Cl, and —CF₃; and
n is an integer from 1-3
to make γ-secretase modulators of Formula XVI

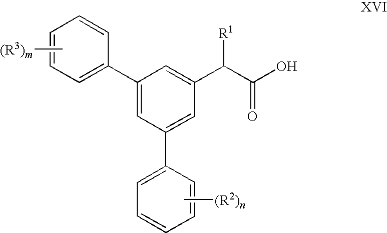

m is an integer from 1-3;
R³ is selected from the group consisting of —H, —C₍₁₋₄₎alkyl, —OC₍₁₋₄₎alkyl, —NO₂, —CN, —NH₂, —F, —Br, —Cl, and —CF₃; and
solvates, hydrates, prodrugs, and pharmaceutically acceptable salts thereof.

In another embodiment of the invention

The invention relates to the use of compounds of Formula I

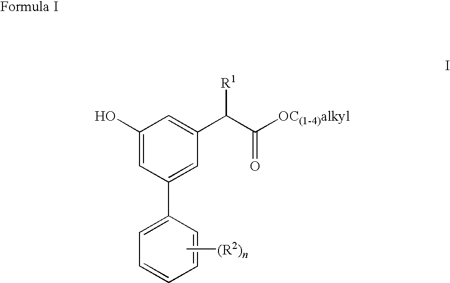

wherein:
R¹ is C₍₁₋₄₎alkyl, or C₍₁₋₅₎alkenyl;
R² is selected from the group consisting of —H, —C₍₁₋₄₎alkyl, —OC₍₁₋₄₎alkyl, —NO₂, —CN, —NH₂, —F, —Br, —Cl, and —CF₃; and
n is an integer from 1-3
to make γ-secretase modulators of Formulas XVII

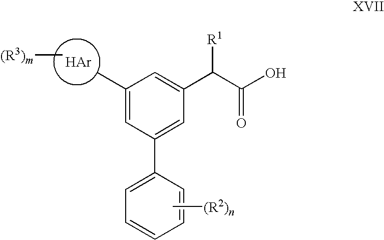

HAr is heteroaryl;
m is an integer from 1-3;
R³ is selected from the group consisting of —H, —C₍₁₋₄₎alkyl, —OC₍₁₋₄₎alkyl, —NO₂, —CN, —NH₂, —F, —Br, —Cl, and —CF₃; and
solvates, hydrates, prodrugs, and pharmaceutically acceptable salts thereof.

In another embodiment of the invention

The invention relates to the use of compounds of Formula I

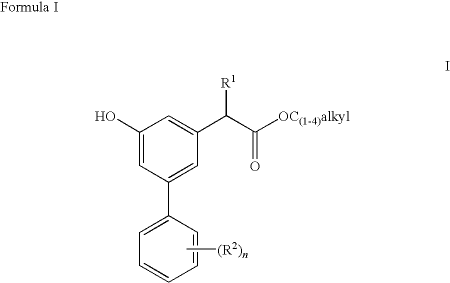

wherein:
R¹ is C₍₁₋₄₎alkyl, or C₍₁₋₅₎alkenyl;
R² is selected from the group consisting of —H, —C₍₁₋₄₎alkyl, —OC₍₁₋₄₎alkyl, —NO₂, —CN, —NH₂, —F, —Br, —Cl, and —CF₃; and
n is an integer from 1-3
to make γ-secretase modulators of Formula XVIII

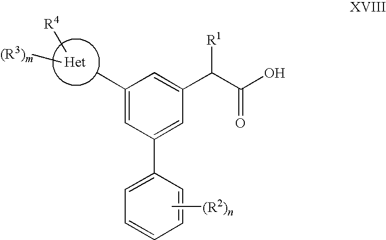

Het is heterocyclyl;
m is an integer from 1-3;
R³ is selected from the group consisting of —H, —C₍₁₋₄₎alkyl, —OC₍₁₋₄₎alkyl, —NO₂, —CN, —NH₂, —F, —Br, —Cl, and —CF₃;
R⁴ is C₍₁₋₄₎alkyl; and
solvates, hydrates, prodrugs, and pharmaceutically acceptable salts thereof.

In another embodiment of the invention

The invention relates to the use of compounds of Formula I

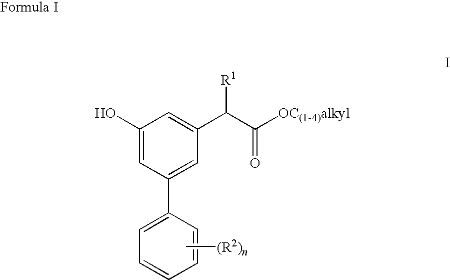

wherein:
R¹ is C₍₁₋₄₎alkyl, or C₍₁₋₅₎alkenyl;
R² is selected from the group consisting of —H, —C₍₁₋₄₎alkyl, —OC₍₁₋₄₎alkyl, —NO₂, —CN, —NH₂, —F, —Br, —Cl, and —CF₃; and
n is an integer from 1-3
to make γ-secretase modulators of Formula XIX

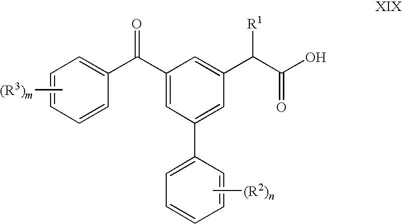

wherein
m is an integer from 1-3;
R³ is selected from the group consisting of —H, —C₍₁₋₄₎alkyl, —OC₍₁₋₄₎alkyl, —NO₂, —CN, —NH₂, —F, —Br, —Cl, and —CF₃; and
solvates, hydrates, prodrugs, and pharmaceutically acceptable salts thereof.

In another embodiment of the invention

The invention relates to the use of compounds of Formula I

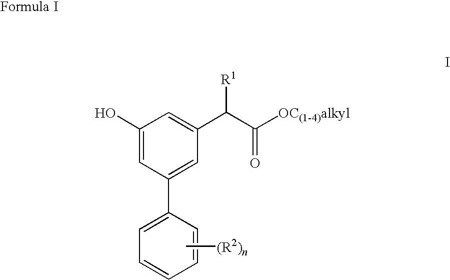

wherein:
R¹ is C₍₁₋₄₎alkyl, or C₍₁₋₅₎alkenyl;
R² is selected from the group consisting of —H, —C₍₁₋₄₎alkyl, —OC₍₁₋₄₎alkyl, —NO₂, —CN, —NH₂, —F, —Br, —Cl, and —CF₃; and
n is an integer from 1-3
to make γ-secretase modulators of Formula XX

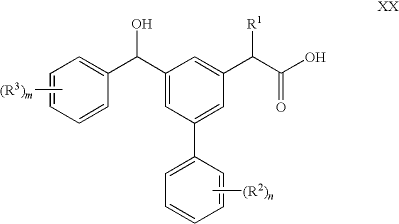

wherein
m is an integer from 1-3;
R³ is selected from the group consisting of —H, —C₍₁₋₄₎alkyl, —OC₍₁₋₄₎alkyl, —NO₂, —CN, —NH₂, —F, —Br, —Cl, and —CF₃; and
solvates, hydrates, prodrugs, and pharmaceutically acceptable salts thereof.

In another embodiment of the invention

The invention relates to the use of compounds of Formula I

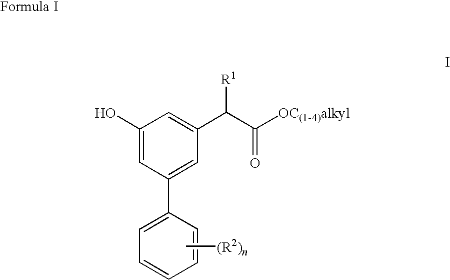

wherein:
R¹ is C₍₁₋₄₎alkyl, or C₍₁₋₅₎alkenyl;
R² is selected from the group consisting of —H, —C₍₁₋₄₎alkyl, —OC₍₁₋₄₎alkyl, —NO₂, —CN, —NH₂, —F, —Br, —Cl, and —CF₃; and
n is an integer from 1-3
to make γ-secretase modulators of Formula XXI

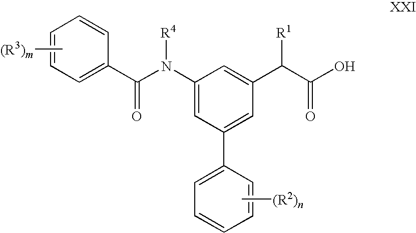

wherein
m is an integer from 1-3;
R³ is selected from the group consisting of —H, —C₍₁₋₄₎alkyl, —OC₍₁₋₄₎alkyl, —NO₂, —CN, —NH₂, —F, —Br, —Cl, and —CF₃;
R⁴ is C₍₁₋₄₎alkyl; and
solvates, hydrates, prodrugs, and pharmaceutically acceptable salts thereof.

In another embodiment of the invention

The invention relates to the use of compounds of Formula I

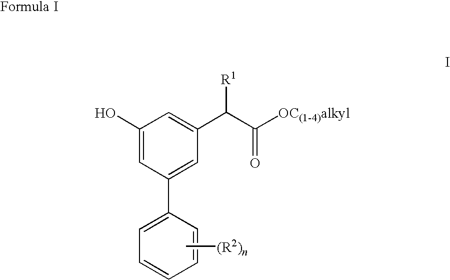

wherein:
R¹ is C₍₁₋₄₎alkyl, or C₍₁₋₅₎alkenyl;
R² is selected from the group consisting of —H, —C₍₁₋₄₎alkyl, —OC₍₁₋₄₎alkyl, —NO₂, —CN, —NH₂, —F, —Br, —Cl, and —CF₃; and
n is an integer from 1-3
to make γ-secretase modulators of Formula XXII

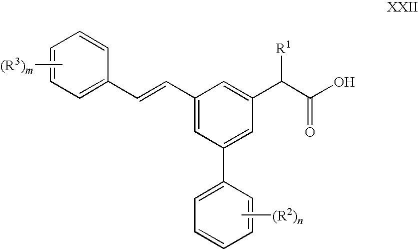

wherein
m is an integer from 1-3;
R³ is selected from the group consisting of —H, —C₍₁₋₄₎alkyl, —OC₍₁₋₄₎alkyl, —NO₂, —CN, —NH₂, —F, —Br, —Cl, and —CF₃; and
solvates, hydrates, prodrugs, and pharmaceutically acceptable salts thereof.

In another embodiment of the invention

The invention relates to the use of compounds of Formula I

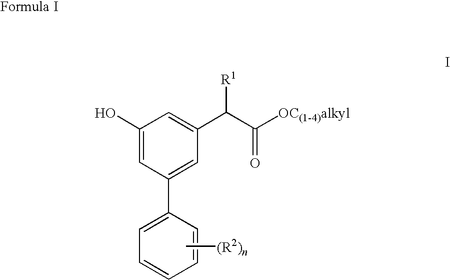

wherein:
R¹ is C₍₁₋₄₎alkyl, or C₍₁₋₅₎alkenyl;
R² is selected from the group consisting of —H, —C₍₁₋₄₎alkyl, —OC₍₁₋₄₎alkyl, —NO₂, —CN, —NH₂, —F, —Br, —Cl, and —CF₃; and
n is an integer from 1-3 to make γ-secretase modulators of Formula XXIII

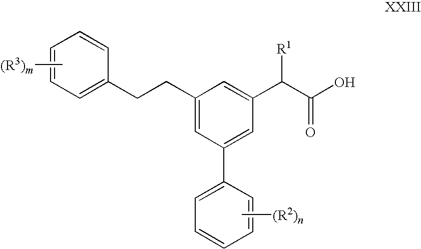

wherein
m is an integer from 1-3;
R³ is selected from the group consisting of —H, —C₍₁₋₄₎alkyl, —OC₍₁₋₄₎alkyl, —NO₂, —CN, —NH₂, —F, —Br, —Cl, and —CF₃; and
solvates, hydrates, prodrugs, and pharmaceutically acceptable salts thereof.

In another embodiment of the invention

The invention relates to the use of compounds of Formula I

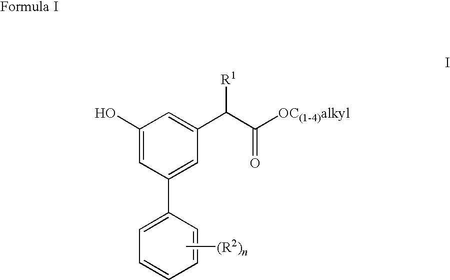

wherein:
R¹ is C₍₁₋₄₎alkyl, or C₍₁₋₅₎alkenyl;
R² is selected from the group consisting of —H, —C₍₁₋₄₎alkyl, —OC₍₁₋₄₎alkyl, —NO₂, —CN, —NH₂, —F, —Br, —Cl, and —CF₃; and
n is an integer from 1-3
to make γ-secretase modulators of Formula XXIV

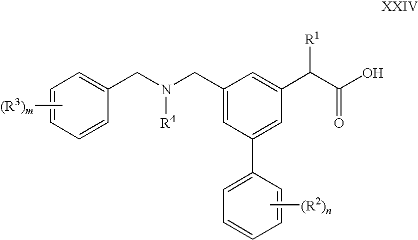

wherein
m is an integer from 1-3;
R³ is selected from the group consisting of —H, —C₍₁₋₄₎alkyl, —OC₍₁₋₄₎alkyl, —NO₂, —CN, —NH₂, —F, —Br, —Cl, and —CF₃;
R⁴ is C₍₁₋₄₎alkyl; and
solvates, hydrates, prodrugs, and pharmaceutically acceptable salts thereof.

In another embodiment of the invention

The invention relates to the use of compounds of Formula I

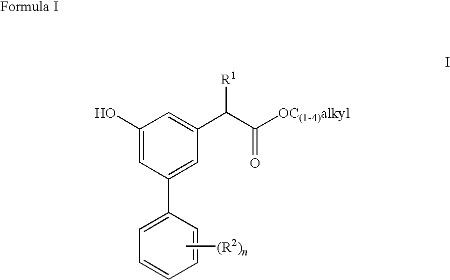

wherein:
R¹ is C₍₁₋₄₎alkyl, or C₍₁₋₅₎alkenyl;
R² is selected from the group consisting of —H, —C₍₁₋₄₎alkyl, —OC₍₁₋₄₎alkyl, —NO₂, —CN, —NH₂, —F, —Br, —Cl, and —CF₃; and
n is an integer from 1-3
to make γ-secretase modulators of Formula XXV

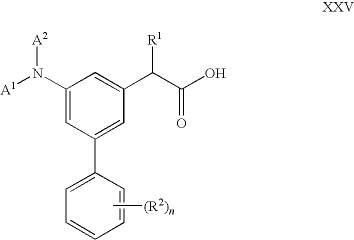

wherein
A¹ is H or —C₍₁₋₄₎alkyl;
A² is —C₍₁₋₄₎alkyl;
alternatively, A¹ and A² may be taken together to form a nitrogen containing heterocyclic ring selected from the following:

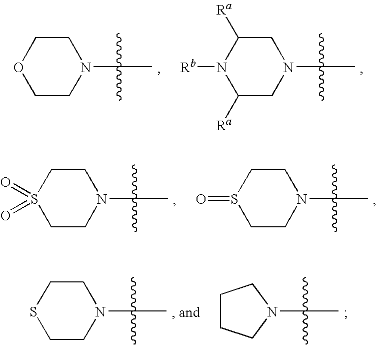

wherein:
R^{a} is H, CH₃, or CH₂CH₃;

### R^{b} is H, or CH₃; and

solvates, hydrates, prodrugs, and pharmaceutically acceptable salts thereof.

In another embodiment of the invention

The invention relates to the use of compounds of Formula I

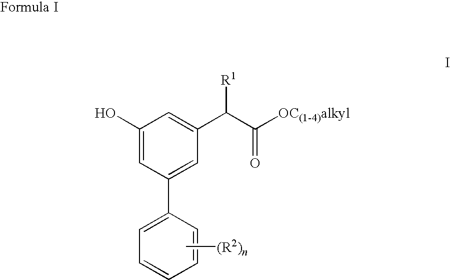

wherein:
R¹ is C₍₁₋₄₎alkyl, or C₍₁₋₅₎alkenyl;
R² is selected from the group consisting of —H, —C₍₁₋₄₎alkyl, —OC₍₁₋₄₎alkyl, —NO₂, —CN, —NH₂, —F, —Br, —Cl, and —CF₃; and
n is an integer from 1-3
to make γ-secretase modulators of Formula XXVI

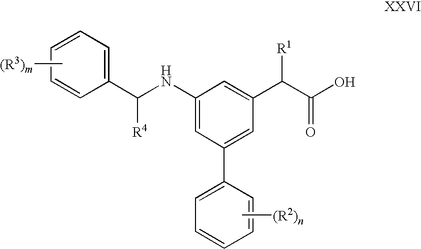

wherein
m is an integer from 1-3;
R³ is selected from the group consisting of —H, —C₍₁₋₄₎alkyl, —OC₍₁₋₄₎alkyl, —NO₂, —CN, —NH₂, —F, —Br, —Cl, and —CF₃;
R⁴ is C₍₁₋₄₎alkyl; and
solvates, hydrates, prodrugs, and pharmaceutically acceptable salts thereof.

In another embodiment of the invention

The invention relates to the use of compounds of Formula I

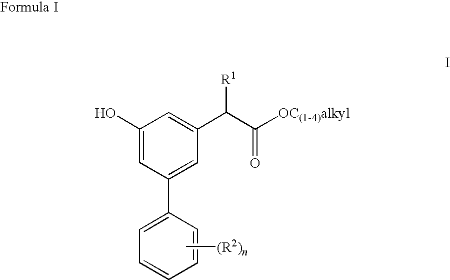

wherein:
R¹ is C₍₁₋₄₎alkyl, or C₍₁₋₅₎alkenyl;
R² is selected from the group consisting of —H, —C₍₁₋₄₎alkyl, —OC₍₁₋₄₎alkyl, —NO₂, —CN, —NH₂, —F, —Br, —Cl, and —CF₃; and
n is an integer from 1-3
to make γ-secretase modulators of Formula XXVII

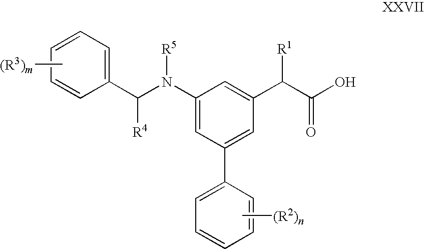

wherein
m is an integer from 1-3;
R³ is selected from the group consisting of —H, —C₍₁₋₄₎alkyl, —OC₍₁₋₄₎alkyl, —NO₂, —CN, —NH₂, —F, —Br, —Cl, and —CF₃;
R⁴ is C₍₁₋₄₎alkyl;
R⁵ is C₍₁₋₄₎alkyl; and
solvates, hydrates, prodrugs, and pharmaceutically acceptable salts thereof.

The invention relates to the use of compounds of Formula I

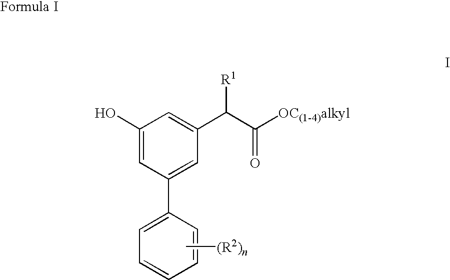

wherein:
R¹ is C₍₁₋₄₎alkyl;
R² is selected from the group consisting of —F, —Br, —Cl, and —CF₃; and
n is an integer from 1-3
to make γ-secretase modulators of Formulas XIII-XXVII

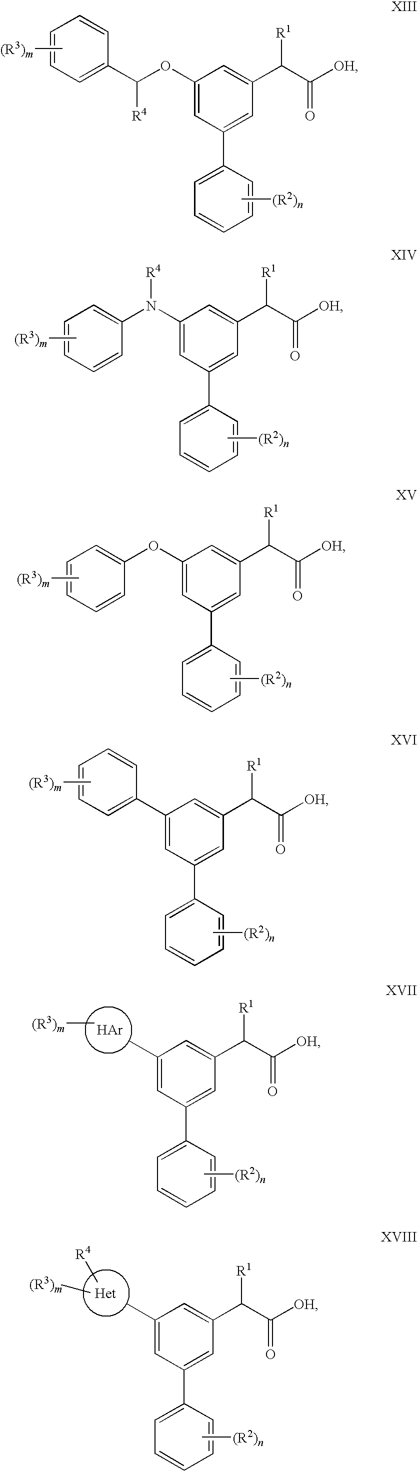

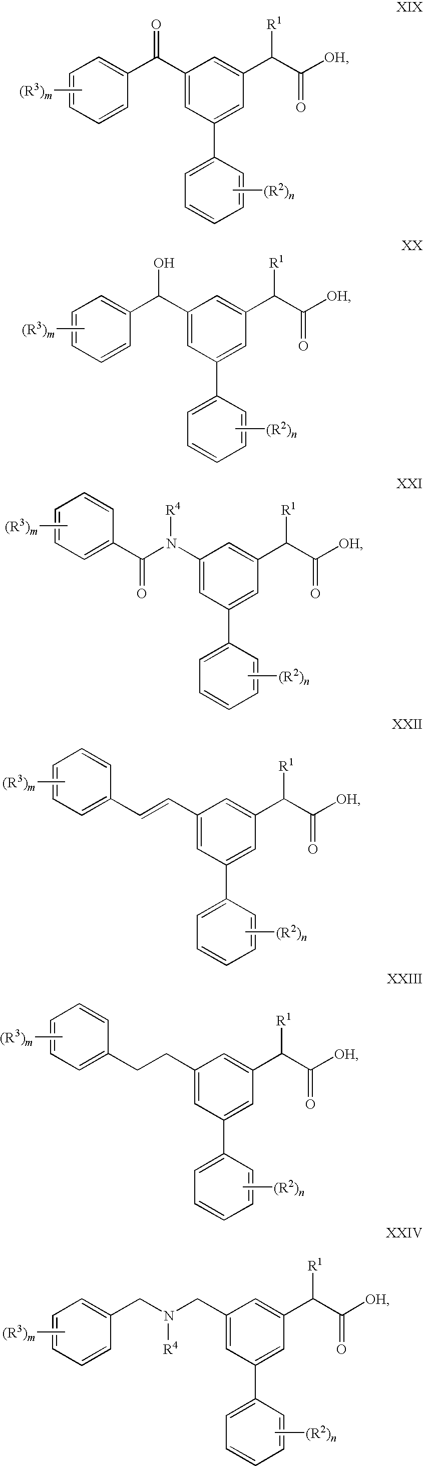

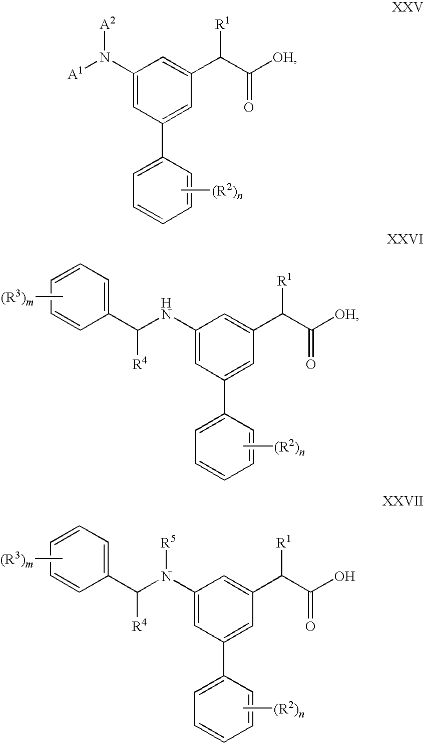

wherein
Het is heterocyclyl;
HAr is heteroaryl;
R³ is selected from the group consisting of —F, —Br, —Cl, and —CF₃;
R⁴ is C₍₁₋₄₎alkyl;
R⁵ is C₍₁₋₄₎alkyl;
A¹ is H or —C₍₁₋₄₎alkyl;
A² is —C₍₁₋₄₎alkyl;
alternatively, A¹ and A² may be taken together to form a nitrogen containing heterocyclic ring selected from the following:

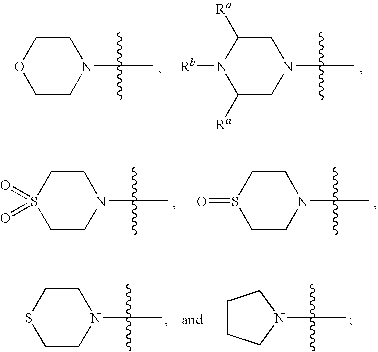

wherein:
R^{a} is H, CH₃, or CH₂CH₃; and
R^{b} is H, or CH₃;
   m is an integer from 1-3;
   n is an integer from 1-3; and
   solvates, hydrates, prodrugs, and pharmaceutically acceptable salts thereof.

In another embodiment of the invention

The invention relates to the use of compounds of Formula I

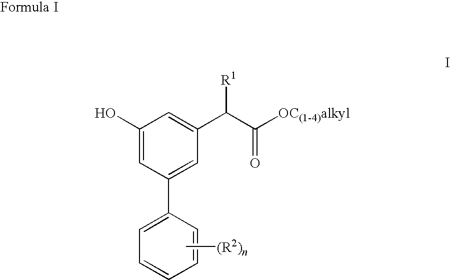

wherein:
R¹ is C₍₁₋₄₎alkyl;
R² is selected from the group consisting of F, Br, Cl, and CF₃; and
n is an integer from 1-3
to make γ-secretase modulators of Formula XIII

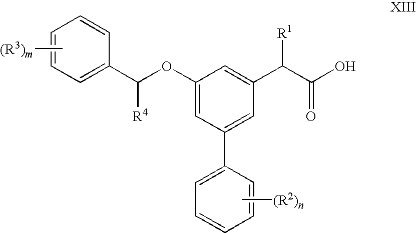

wherein
m is an integer from 1-3;
R³ is selected from the group consisting of F, Br, Cl, and CF₃;
R⁴ is C₍₁₋₄₎alkyl; and
solvates, hydrates, prodrugs, and pharmaceutically acceptable salts thereof.

In another embodiment of the invention

The invention relates to the use of compounds of Formula I

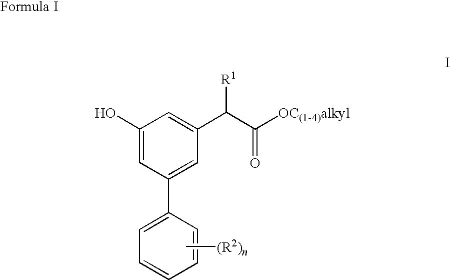

wherein:
R¹ is C₍₁₋₄₎alkyl;
R² is selected from the group consisting of F, Br, Cl, and CF₃; and
n is an integer from 1-3
to make γ-secretase modulators of Formula XIV

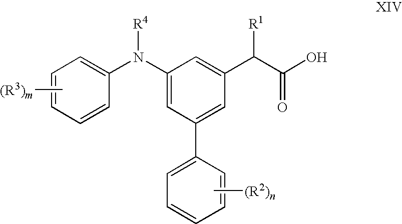

wherein
m is an integer from 1-3;
R³ is selected from the group consisting of F, Br, Cl, and CF₃;
R⁴ is C₍₁₋₄₎alkyl; and
solvates, hydrates, prodrugs, and pharmaceutically acceptable salts thereof.

In another embodiment of the invention

The invention relates to the use of compounds of Formula I

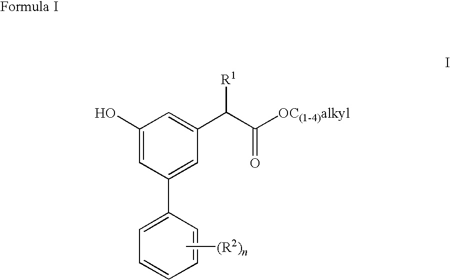

wherein:
R¹ is C₍₁₋₄₎alkyl;
R² is selected from the group consisting of F, Br, Cl, and CF₃; and
n is an integer from 1-3
to make γ-secretase modulators of Formula XV

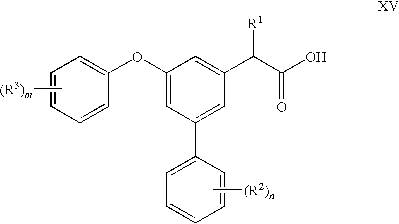

wherein
m is an integer from 1-3;
R³ is selected from the group consisting of F, Br, Cl, and CF₃; and
solvates, hydrates, prodrugs, and pharmaceutically acceptable salts thereof.

In another embodiment of the invention

The invention relates to the use of compounds of Formula I

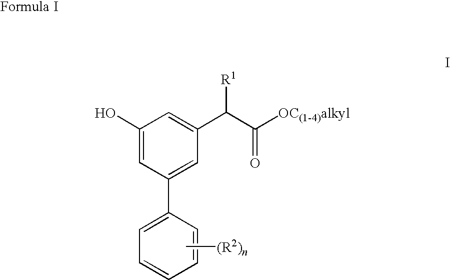

wherein:
R¹ is C₍₁₋₄₎alkyl;
R² is selected from the group consisting of F, Br, Cl, and CF₃; and
n is an integer from 1-3
to make γ-secretase modulators of Formula XVI

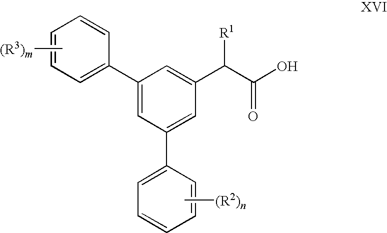

m is an integer from 1-3;
R³ is selected from the group consisting of F, Br, Cl, and CF₃; and
solvates, hydrates, prodrugs, and pharmaceutically acceptable salts thereof.

In another embodiment of the invention

The invention relates to the use of compounds of Formula I

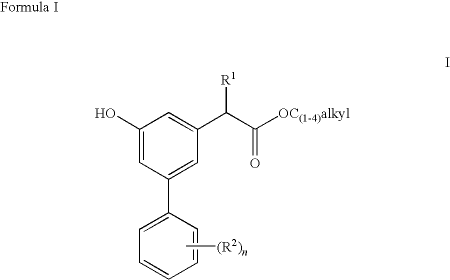

wherein:
R¹ is C₍₁₋₄₎alkyl;
R² is selected from the group consisting of F, Br, Cl, and CF₃; and
n is an integer from 1-3
to make γ-secretase modulators of Formulas XVII

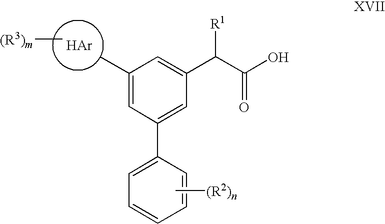

HAr is heteroaryl;
m is an integer from 1-3;
R³ is selected from the group consisting of F, Br, Cl, and CF₃; and
solvates, hydrates, prodrugs, and pharmaceutically acceptable salts thereof.

In another embodiment of the invention

The invention relates to the use of compounds of Formula I

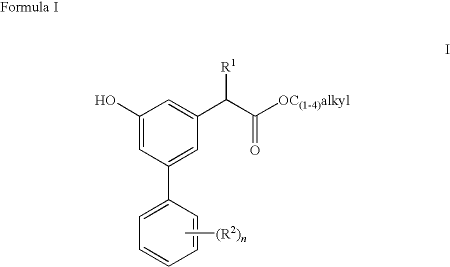

wherein:
R¹ is C₍₁₋₄₎alkyl;
R² is selected from the group consisting of F, Br, Cl, and CF₃; and
n is an integer from 1-3
to make γ-secretase modulators of Formula XVIII

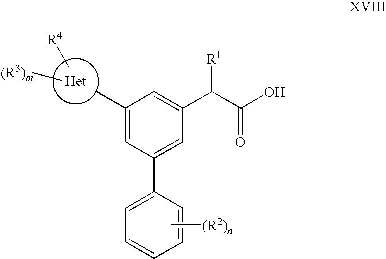

Het is heterocyclyl;
m is an integer from 1-3;
R³ is selected from the group consisting of F, Br, Cl, and CF₃;
R⁴ is C₍₁₋₄₎alkyl; and
solvates, hydrates, prodrugs, and pharmaceutically acceptable salts thereof.

In another embodiment of the invention

The invention relates to the use of compounds of Formula I

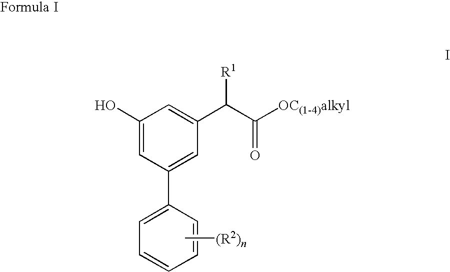

wherein:
R¹ is C₍₁₋₄₎alkyl;
R² is selected from the group consisting of F, Br, Cl, and CF₃; and
n is an integer from 1-3
to make γ-secretase modulators of Formula XIX

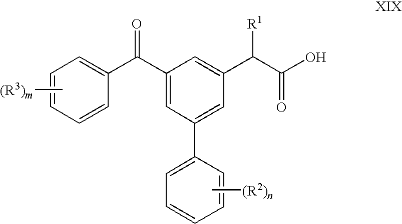

wherein
m is an integer from 1-3;
R³ is selected from the group consisting of F, Br, Cl, and CF₃; and
solvates, hydrates, prodrugs, and pharmaceutically acceptable salts thereof.

In another embodiment of the invention

The invention relates to the use of compounds of Formula I

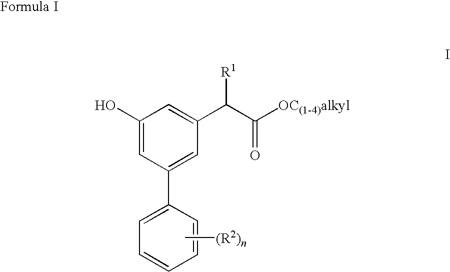

wherein:
R¹ is C₍₁₋₄₎alkyl;
R² is selected from the group consisting of F, Br, Cl, and CF₃; and
n is an integer from 1-3
to make γ-secretase modulators of Formula XX

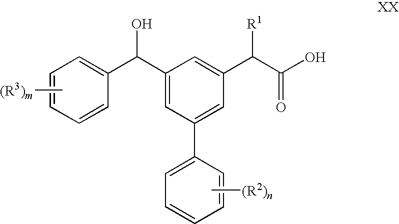

wherein
m is an integer from 1-3;
R³ is selected from the group consisting of F, Br, Cl, and CF₃; and
solvates, hydrates, prodrugs, and pharmaceutically acceptable salts thereof.

In another embodiment of the invention

The invention relates to the use of compounds of Formula I

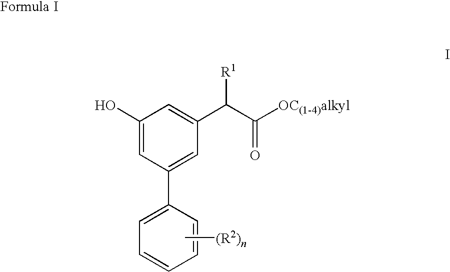

wherein:
R¹ is C₍₁₋₄₎alkyl;
R² is selected from the group consisting of F, Br, Cl, and CF₃; and
n is an integer from 1-3
to make γ-secretase modulators of Formula XXI

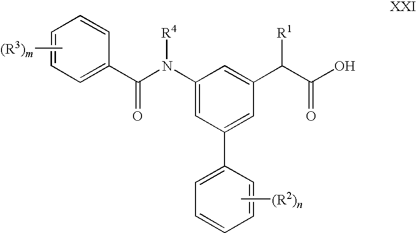

wherein
m is an integer from 1-3;
R³ is selected from the group consisting of F, Br, Cl, and CF₃;
R⁴ is C₍₁₋₄₎alkyl; and
solvates, hydrates, prodrugs, and pharmaceutically acceptable salts thereof.

In another embodiment of the invention

The invention relates to the use of compounds of Formula I

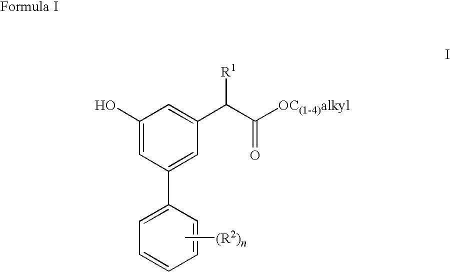

wherein:
R¹ is C₍₁₋₄₎alkyl;
R² is selected from the group consisting of F, Br, Cl, and CF₃; and
n is an integer from 1-3
to make γ-secretase modulators of Formula XXII

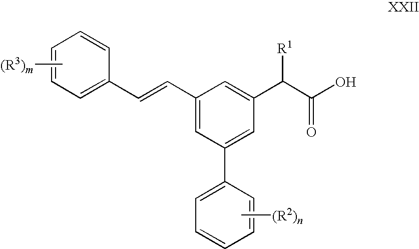

wherein
m is an integer from 1-3;
R³ is selected from the group consisting of F, Br, Cl, and CF₃; and
solvates, hydrates, prodrugs, and pharmaceutically acceptable salts thereof.

In another embodiment of the invention

The invention relates to the use of compounds of Formula I

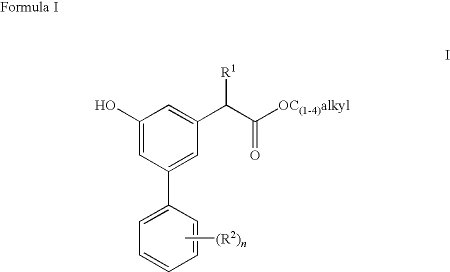

wherein:
R¹ is C₍₁₋₄₎alkyl;
R² is selected from the group consisting of F, Br, Cl, and CF₃; and
n is an integer from 1-3
to make γ-secretase modulators of Formula XXIII

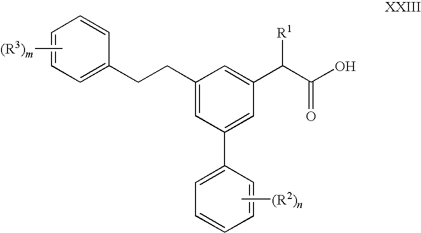

wherein
m is an integer from 1-3;
R³ is selected from the group consisting of F, Br, Cl, and CF₃; and
solvates, hydrates, prodrugs, and pharmaceutically acceptable salts thereof.

In another embodiment of the invention

The invention relates to the use of compounds of Formula I

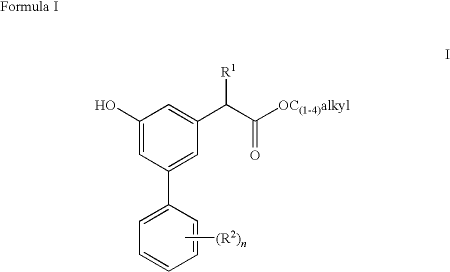

wherein:
R¹ is C₍₁₋₄₎alkyl;
R² is selected from the group consisting of F, Br, Cl, and CF₃; and
n is an integer from 1-3
to make γ-secretase modulators of Formula XXIV

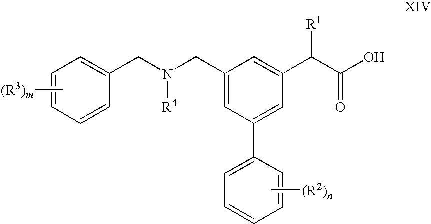

wherein
m is an integer from 1-3;
R³ is selected from the group consisting of F, Br, Cl, and CF₃;
R⁴ is C₍₁₋₄₎alkyl; and
solvates, hydrates, prodrugs, and pharmaceutically acceptable salts thereof.

In another embodiment of the invention

The invention relates to the use of compounds of Formula I

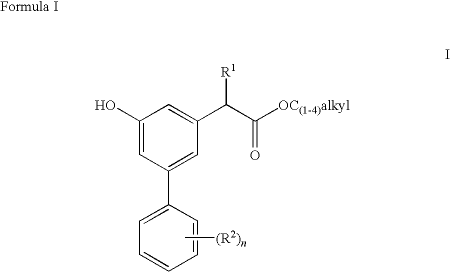

wherein:
R¹ is C₍₁₋₄₎alkyl;
R² is selected from the group consisting of F, Br, Cl, and CF₃; and
n is an integer from 1-3
to make γ-secretase modulators of Formula XXV

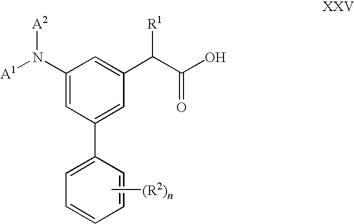

wherein
A¹ is H or —C₍₁₋₄₎alkyl;
A² is —C₍₁₋₄₎alkyl;
alternatively, A¹ and A² may be taken together to form a nitrogen containing heterocyclic ring selected from the following:

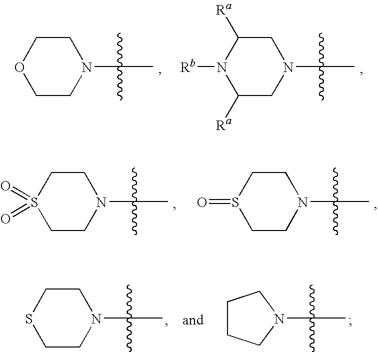

wherein:
R^{a} is H, CH₃, or CH₂CH₃;

### R^{b} is H, or CH₃; and

solvates, hydrates, prodrugs, and pharmaceutically acceptable salts thereof.

In another embodiment of the invention

The invention relates to the use of compounds of Formula I

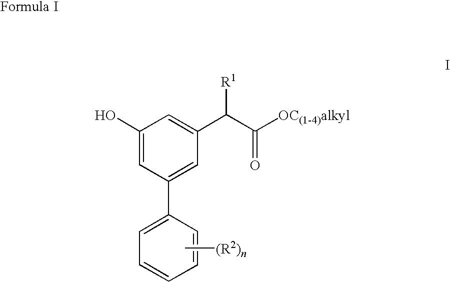

wherein:
R¹ is C₍₁₋₄₎alkyl;
R² is selected from the group consisting of F, Br, Cl, and CF₃; and
n is an integer from 1-3
to make γ-secretase modulators of Formula XXVI

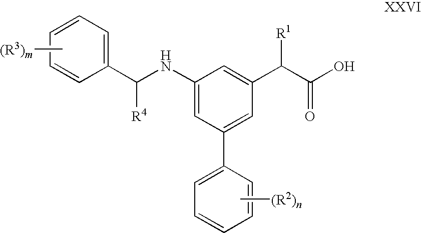

wherein
m is an integer from 1-3;
R³ is selected from the group consisting of F, Br, Cl, and CF₃;
R⁴ is C₍₁₋₄₎alkyl; and
solvates, hydrates, prodrugs, and pharmaceutically acceptable salts thereof.

In another embodiment of the invention

The invention relates to the use of compounds of Formula I

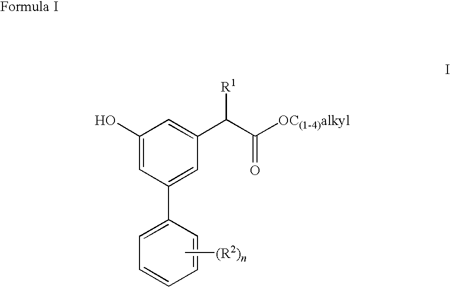

wherein:
R¹ is C₍₁₋₄₎alkyl;
R² is selected from the group consisting of F, Br, Cl, and CF₃; and
n is an integer from 1-3
to make γ-secretase modulators of Formula XXVII

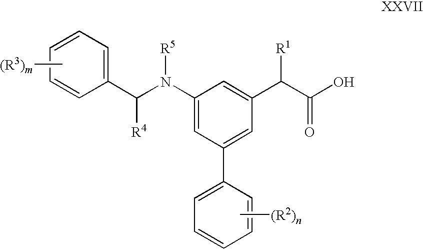

wherein
m is an integer from 1-3;
R³ is selected from the group consisting of F, Br, Cl, and CF₃;
R⁴ is C₍₁₋₄₎alkyl;
R⁵ is C₍₁₋₄₎alkyl; and
solvates, hydrates, prodrugs, and pharmaceutically acceptable salts thereof.

In another embodiment:

The invention relates to a method of selective mono-debenzylation of a 3,5 bis-benzyloxy moiety, such as:

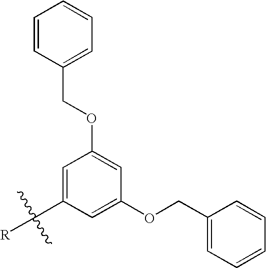

characterized by the use of 0.9 to 1.1 equivalents of a base selected from the group consisting of NaH, KH, NaOH, KOH, LiOH, KOtBu, NaOtBu, K₂CO₃, Na₂CO₃, Cs₂CO₃ and NaN(Si(CH₃)₃)₂ or LiN(Si(CH₃)₃)₂, and one equivalent of hydrogen.

One skilled in the art will recognize that the compounds of Formula I may have one or more asymmetric carbon atoms in their structure. It is intended that the present invention include within its scope single enantiomer forms of the compounds, racemic mixtures, and mixtures of enantiomers in which an enantiomeric excess is present.

Some of the compounds of the inventions and/or salts or esters thereof will exist in different stereoisomeric forms. All of these forms are subjects of the invention.

Described below are exemplary salts of the compounds according to the invention which are included herein. The list of the different salts stated below is not meant to be complete and limiting.

Compounds according to the invention which contain one or more acidic groups can be used according to the invention, e.g. as their alkali metal salts, alkaline earth metal salts or ammonium salts. More precise examples of such salts include sodium salts, potassium salts, calcium salts, magnesium salts or salts with ammonia or organic amines such as, e.g. ethylamine, ethanolamine, triethanolamine or amino acids.

The term “pharmaceutically acceptable” means approved by a regulatory agency such as the EMEA (Europe) and/or the FDA (US) and/or any other national regulatory agency for use in animals, preferably in humans.

The respective salts of the compounds according to the invention can be obtained by customary methods which are known to the person skilled in the art, for example by contacting these with an organic or inorganic base in a solvent or dispersant, or by cation exchange with other salts.

Furthermore, the invention includes all salts of the compounds according to the invention which, owing to low physiological compatibility, are not directly suitable for use in pharmaceuticals but which can be used, for example, as intermediates for chemical reactions or for the preparation of pharmaceutically acceptable salts or which might be suitable for studying γ-secretase modulating activity of a compound according of the invention in any suitable manner, such as any suitable in vitro assay.

The invention is considered to include prodrugs, i.e., derivatives of an acting drug that possess superior delivery capabilities and therapeutic value as compared to the acting drug. Prodrugs are transformed into active drugs by in vivo enzymatic or chemical processes.

The present invention furthermore includes all solvates of the compounds according to the invention.

The present invention furthermore includes derivatives/prodrugs (including the salts thereof) of the compounds according to the invention which contain physiologically tolerable and cleavable groups and which are metabolized in animals, preferably mammals, most preferably humans into a compound according to the invention.

The present invention furthermore includes the metabolites of the compounds according to the invention.

The term “metabolites” refers to all molecules derived from any of the compounds according to the invention in a cell or organism, preferably mammal.

Preferably the term “metabolites” relates to molecules which differ from any molecule which is present in any such cell or organism under physiological conditions.

The structure of the metabolites of the compounds according to the invention will be obvious to any person skilled in the art, using the various appropriate methods.

The invention also relates to compounds of the invention for use as medicaments. The compounds are as defined above, furthermore with respect to the medicaments the embodiments as described below with respect to the use of the invention, e.g. formulation, application and combination, also apply to this aspect of the invention.

In particular the compounds according to the invention are suitable for the treatment of Alzheimer's disease.

Details relating to said use are further disclosed below.

The compounds can be used for modulation of γ-secretase activity.

As used herein, the term “modulation of γ-secretase activity” refers to an effect on the processing of APP by the γ-secretase-complex. Preferably it refers to an effect in which the overall rate of processing of APP remains essentially as without the application of said compounds, but in which the relative quantities of the processed products are changed, more preferably in such a way that the amount of the Aβ42-peptide produced is reduced. For example a different Abeta species can be produced (e.g. Abeta-38 or other Abeta peptide species of shorter amino acid sequence instead of Abeta-42) or the relative quantities of the products are different (e.g. the ratio of Abeta-40 to Abeta-42 is changed, preferably increased).

Gamma secretase activity can e.g. be measured by determining APP processing, e.g. by determining the levels of Abeta peptide species produced, most importantly levels of Abeta-42 (see Example section, infra).

It has been previously shown that the γ-secretase complex is also involved in the processing of the Notch-protein. Notch is a signaling protein which plays a crucial role in developmental processes (e.g. reviewed in Schweisguth F (2004) Curr. Biol. 14, R129). With respect to the use of said compounds for the modulation of γ-secretase activity in therapy, it seems particularly advantageous not to interfere with the Notch-processing activity of the γ-secretase activity in order to avoid putative undesired side-effects. Thus, compounds are preferred which do not show an effect on the Notch-processing activity of the γ-secretase-complex.

Within the meaning of the invention, “effect on the Notch processing activity” includes both an inhibition or an activation of the Notch-processing activity by a certain factor. A compound is defined as not having an effect on the Notch processing activity, if said factor is smaller than 20, preferably smaller than 10, more preferably smaller than 5, most preferably smaller than 2 in the respective assay as described in Shimizu et al (2000) Mol. Cell. Biol, 20: 6913 at a concentration of 30 μM.

Such a γ-secretase modulation can be carried out, e.g. in animals such as mammals. Exemplary mammals are mice, rats, guinea pigs, monkeys, dogs, cats. The modulation can also be carried out in humans. In a particular embodiment of the invention, said modulation is performed in vitro or in cell culture. As known to the person skilled in the art, several in vitro and cell culture assays are available.

Exemplary assays useful for measuring the production of C-terminal APP fragments in cell lines or transgenic animals by Western blot analysis include but are not limited to those described in Yan et al., 1999, Nature 402, 533-537.

An example of an in vitro γ-secretase assay is described in WO-03/008635. In this assay a suitable peptide substrate is contacted with a γ-secretase preparation and the ability to cleave the substrate is measured.

Concentrations of the various products of the γ-secretase cleavage (the Aβ-peptides) can be determined by various methods known to a person skilled in the art. Examples for such methods include determination of the peptides by mass-spectrometry or detection by antibodies.

Exemplary assays useful for the characterization of the profile of soluble Aβ peptides in cultured cell media and biological fluids include but are not limited to those described by Wang et al., 1996, J. Biol. Chem. 271, 31894-31902. In this assay a combination of immunoprecipitation of Abeta-peptides with specific antibodies and detection and quantification of the peptide species with matrix-assisted laser desorption ionization time-of-flight mass spectrometry is used.

Exemplary assays useful for measuring the production of Abeta-40 and Abeta-42 peptides by ELISA include but are not limited to those described in Vassar et al, 1999, Science 286, 735-741. Further information is disclosed for example in N. Ida et al. (1996) J. Biol. Chem. 271, 22908, and M. Jensen et al. (2000) Mol. Med. 6, 291. Suitable antibodies are available for example from The Genetics Company, Inc., Switzerland. Antibody-based kits are also available from Innogenetics, Belgium.

Cells which can be employed in such assays include cells which endogenously express the γ-secretase complex and transfected cells which transiently or stably express some or all interactors of the γ-secretase complex. Numerous available cell lines suitable for such assays are known to the skilled person. Cells and cell lines of neuronal or glial origin are particularly suitable. Furthermore, cells and tissues of the brain as well as homogenates and membrane preparations thereof may be used (Xia et al., 1998, Biochemistry 37, 16465-16471).

Such assays might be carried out for example to study the effect of the compounds according to the invention in different experimental conditions and configurations.

Furthermore, such assays might be carried out as part of functional studies on the γ-secretase complex.

For example, either one or more interactors (either in their wild-type form or carrying certain mutations and/or modifications) of the γ-secretase complex of an animal, preferably a mammal, more preferably humans, might be expressed in certain cell lines and the effect of the compounds according to the invention might be studied.

Mutated forms of the interactor(s) used can either be mutated forms which have been described in certain animals, preferably mammals, more preferably humans or mutated forms which have not previously been described in said animals.

Modifications of the interactors of the γ-secretase complex include both any physiological modification of said interactors and other modifications which have been described as modifications of proteins in a biological system.

Examples of such modifications include, but are not limited to, glycosylation, phosphorylation, prenylation, myristylation and farnesylation.

Furthermore, the compounds according to the invention can be used for the preparation of a medicament for the modulation of γ-secretase activity.

The invention further relates to the use of compounds of Formulas XIII-XXVII for the preparation of a medicament for the modulation of γ-secretase activity.

The activity of the γ-secretase can be modulated in different ways, i.e. resulting in different profiles of the various Aβ-peptides.

Respective dosages, routes of administration, formulations etc are disclosed further below.

The invention further relates to the use of the compounds of Formula I to synthesize compounds of Formulas XIII-XXVII for the treatment of a disease associated with an elevated level of Aβ42-production. The disease with elevated levels of Abeta peptide production and deposition in the brain is typically Alzheimer's disease (AD), cerebral amyloid angiopathy, multi-infarct dementia, dementia pugilistica or Down syndrome, preferably AD.

As used herein, the term “treatment” is intended to refer to all processes, wherein there may be a slowing, interrupting, arresting, or stopping of the progression of a disease, but does not necessarily indicate a total elimination of all symptoms.

As used herein, the term “elevated level of Aβ42-production” refers to a condition in which the rate of production of Aβ42-peptide is increased due to an overall increase in the processing of APP or, preferably, it refers to a condition in which the production of the Aβ42 peptide is increased due to a modification of the APP-processing profile in comparison to the wild-type APP and non-pathological situation.

As outlined above, such an elevated Aβ42-level is a hallmark of patients developing or suffering from Alzheimer's disease.

One advantage of the compounds or a part of the compounds of the present invention may lie in their enhanced CNS-penetration.

Furthermore the invention relates to a pharmaceutical composition comprising a compound of Formula XIII-XXVII in a mixture with an inert carrier.

Modulators of γ-secretase derived from compounds of Formula I can be formulated into pharmaceutical compositions comprising a compound of Formula XIII-XXVII in a mixture with an inert carrier, where said inert carrier is a pharmaceutical carrier.

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, including but not limited to peanut oil, soybean oil, mineral oil, sesame oil and the like. Water is a preferred carrier when the pharmaceutical composition is administered orally. Saline and aqueous dextrose are preferred carriers when the pharmaceutical composition is administered intravenously. Saline solutions and aqueous dextrose and glycerol solutions are preferably employed as liquid carriers for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsions, tablets, pills, capsules, powders, sustained-release formulations and the like.

The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the patient. The formulation should suit the mode of administration.

The compounds according to the invention and their pharmaceutically acceptable salts, optionally in combination with other pharmaceutically active compounds are suitable to treat or prevent Alzheimer's disease or the symptoms thereof. Such additional compounds include cognition-enhancing drugs such as acetylcholinesterase inhibitors (e.g. Donepezil, Tacrine, Galantamine, Rivastigmin), NMDA antagonists (e.g. Memantine) PDE4 inhibitors (e.g. Ariflo) or any other drug known to a person skilled in the art suitable to treat or prevent Alzheimer's disease. Such compounds also include cholesterol-lowering drugs such as statins (e.g. simvastatin). These compounds can be administered to animals, preferably to mammals, and in particular humans, as pharmaceuticals by themselves, in mixtures with one another or in the form of pharmaceutical preparations.

Various delivery systems are known and can be used to administer a compound of the invention for the treatment of Alzheimer's disease or for the modulation of the γ-secretase activity, e.g. encapsulation in liposomes, microparticles, and microcapsules: If not delivered directly to the central nervous system, preferably the brain, it is advantageous to select and/or modify methods of administration in such a way as to allow the pharmaceutical compound to cross the blood-brain barrier.

Methods of introduction include, but are not limited to, intradermal, intramuscular, intraperitoneal, intravenous, subcutaneous, intranasal, epidural, and oral routes.

The compounds may be administered by any convenient route, for example by infusion, by bolus injection, by absorption through epithelial or mucocutaneous linings and may be administered together with other biologically active agents.

Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g. by use of an inhaler or nebulizer, and formulation with an aerosolizing agent.

Modulators of γ-secretase derived from compounds of Formula I can be delivered in a vesicle, in particular a liposome (Langer (1990) Science 249, 1527.

Modulators of γ-secretase derived from compounds of Formula I can be delivered via a controlled release system. In one embodiment, a pump may be used (Sefton (1987) CRC Crit. Ref. Biomed. Eng. 14, 201; Buchwald et al. (1980) Surgery 88, 507; Saudek et al. (1989) N. Engl. J. Med. 321, 574). In another embodiment, polymeric materials can be used (Ranger and Peppas (1983) Macromol. Sci. Rev. Macromol. Chem. 23, 61; Levy et al. (1985) Science 228, 190; During et al. (1989) Ann. Neurol. 25, 351; Howard et al. (1989) J. Neurosurg. 71, 858). In yet another embodiment, a controlled release system can be placed in proximity of the therapeutic target, i.e., the brain, thus requiring only a fraction of the systemic dose (e.g. Goodson, 1984, In: Medical Applications of Controlled Release, supra, Vol. 2, 115). Other controlled release systems are discussed in the review by Langer (1990, Science 249, 1527).

In order to select an appropriate way of administration, the person skilled in the art will also consider routes of administration which have been selected for other known Anti-Alzheimer-drugs.

For example, Aricept/Donepezil and Cognex/Tacrine (all acetylcholinesterase-inhibitors) are being taken orally, Axura/Memantine (an NMDA-receptor antagonist) has been launched both as tablets/liquid and as an i.v.-solution.

Furthermore, the skilled person in the art will take into account the available data with respect to routes of administration of members of the NSAID-family in clinical trials and other studies investigating their effect on Alzheimer's disease.

In order to select the appropriate dosage, the person skilled in the art will choose a dosage which has been shown to be not toxic in preclinical and/or clinical studies and which can be in accordance with the values given beforehand, or which may deviate from these.

The precise dose to be employed in the formulation will also depend on the route of administration, and the seriousness of the disease or disorder, and should be decided according to the judgment of the practitioner and each patient's circumstances. However, suitable dosage ranges for intravenous administration are generally about 20-500 micrograms of active compound per kilogram body weight. Suitable dosage ranges for intranasal administration are generally about 0.01 mg/kg body weight to 1 mg/kg body weight. Effective doses may be extrapolated from dose-response curves derived from in vitro or animal model test systems.

An exemplary animal model is the transgenic mouse strain “Tg2576” containing an APP695-form with the double mutation KM670/671NL. For reference see e.g. U.S. Pat. No. 5,877,399 and Hsiao et al. (1996) Science 274, 99 and also Kawarabayahsi T (2001) J. Neurosci. 21, 372; Frautschy et al. (1998) Am. J. Pathol. 152, 307; Irizarry et al. (1997) J. Neuropathol. Exp. Neurol. 56, 965; Lehman et al. (2003) Neurobiol. Aging 24, 645.

Substantial data from several studies are available to the skilled person in the art, which are instructive to the skilled person to select the appropriate dosage for the chosen therapeutic regimen.

Numerous studies have been published in which the effects of molecules on the γ-secretase activity are described. Exemplary studies are Lim et al. (2001) Neurobiol. Aging 22, 983; Lim et al. (2000) J. Neurosci. 20, 5709; Weggen et al. (2001) Nature 414, 212; Eriksen et al. (2003) J Clin Invest. 112, 440; Yan et al. (2003) J. Neurosci. 23, 7504.

### DEFINITIONS

The term “alkenyl,” whether used alone or as part of a substituent group, for example, “C₁₋₄alkenyl(aryl),” refers to a partially unsaturated branched or straight chain monovalent hydrocarbon radical having at least one carbon-carbon double bond, whereby the double bond is derived by the removal of one hydrogen atom from each of two adjacent carbon atoms of a parent alkyl molecule and the radical is derived by the removal of one hydrogen atom from a single carbon atom. Atoms may be oriented about the double bond in either the cis (Z) or trans (E) conformation. Typical alkenyl radicals include, but are not limited to, ethenyl, propenyl, allyl (2-propenyl), butenyl and the like. Examples include C₂₋₈alkenyl or C₂₋₄alkenyl groups.

The term “C_{a-b}” (where a and b are integers referring to a designated number of carbon atoms) refers to an alkyl, alkenyl, alkynyl, alkoxy or cycloalkyl radical or to the alkyl portion of a radical in which alkyl appears as the prefix root containing from a to b carbon atoms inclusive. For example, C₁₋₄ denotes a radical containing 1, 2, 3 or 4 carbon atoms.

The term “alkyl” refers to both linear and branched chain radicals of up to 12 carbon atoms, preferably up to 6 carbon atoms, unless otherwise indicated, and includes, but is not limited to, methyl, ethyl, propyl, isopropyl, n-butyl, isobutyl, sec-butyl, tert-butyl, pentyl, isopentyl, hexyl, isohexyl, heptyl, octyl, 2,2,4-trimethylpentyl, nonyl, decyl, undecyl and dodecyl.

The term “heteroaryl” refers to 5- to 7-membered mono- or 8- to 10-membered bicyclic aromatic ring systems, any ring of which may consist of from one to four heteroatoms selected from N, O or S where the nitrogen and sulfur atoms can exist in any allowed oxidation state. Examples include benzimidazolyl, benzothiazolyl, benzothienyl, benzoxazolyl, furyl, imidazolyl, isothiazolyl, isoxazolyl, oxazolyl, pyrazinyl, pyrazolyl, pyridyl, pyrimidinyl, pyrrolyl, quinolinyl, thiazolyl and thienyl.

The term “heterocyclyl” refers to a saturated or partially unsaturated monocyclic ring radical derived by the removal of one hydrogen atom from a single carbon or nitrogen ring atom. Typical heterocyclyl radicals include 2H-pyrrolyl, 2-pyrrolinyl, 3-pyrrolinyl, pyrrolidinyl, 1,3-dioxolanyl, 2-imidazolinyl (also referred to as 4,5-dihydro-1H-imidazolyl), imidazolidinyl, 2-pyrazolinyl, pyrazolidinyl, tetrazolyl, piperidinyl, 1,4-dioxanyl, morpholinyl, 1,4-dithianyl, thiomorpholinyl, piperazinyl, azepanyl, hexahydro-1,4-diazepinyl and the like.

The term “substituted,” refers to a core molecule on which one or more hydrogen atoms have been replaced with one or more functional radical moieties. Substitution is not limited to a core molecule, but may also occur on a substituent radical, whereby the substituent radical becomes a linking group.

### General Schemes Part I

### Synthesis of Compounds of Formula I

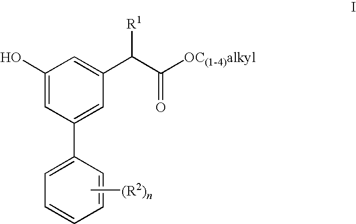

Compounds of Formula I may be prepared by debenzylation of compounds of Formula II by hydrogenation in alcohol, such as MeOH or EtOH, in the presence of Pd—C. Debenzylation can also be achieved with other methods, such as BBr₃ in DCM at room temperature, NaCN in DMSO/120-200° C., or LiCl in DMF/120-200° C.

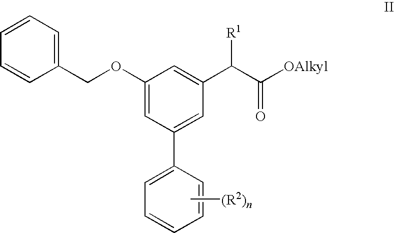

Compounds of Formula II may be prepared from alkylation of compounds of Formula III with an appropriate alkyl bromide, including either sec-butyl bromide or sec-butenyl bromide. Treatment of compounds of Formula III in THF or other aprotic solvent with a base, such as lithium bis(trimethylsilyl)amide, sodium bis(trimethylsilyl)amide, or lithium diisopropylamide at −78° C., followed by the addition of an alkyl bromide yields alkylated II.

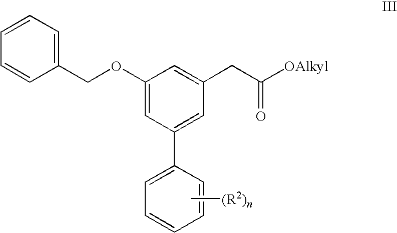

Compounds of Formula III may be prepared from IV through a coupling reaction with an arylboronic acid under Suzuki conditions of aqueous sodium carbonate in DME in the presence of Pd(PPh₃)₄.

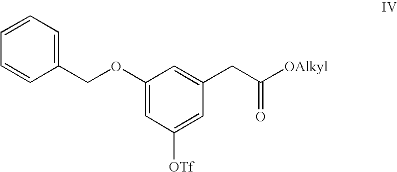

Intermediate IV may be prepared from compounds of Formula V with trifluoromethanesulfonic anhydride in DCM, in the presence of one equivalent of pyridine at 0° C., or the triflate can be prepared from V with N-phenyl-bis-(trifluoromethanesulfonimide) and triethylamine in THF at reflux.

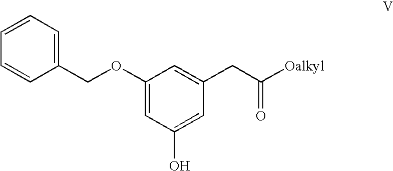

Intermediate phenolic ester V can be prepared from mono-debenzylation of VI. Selective mono-debenzylation of VI can be achieved by hydrogenation with 1.1 equivalents of base, e.g. sodium hydroxide or potassium hydroxide, in ethanol or methanol solution in the presence of Pd—C catalyst under hydrogen atmosphere (<60 psi) in a Parr shaker. The reaction is allowed to proceed until one equivalent of hydrogen is consumed.

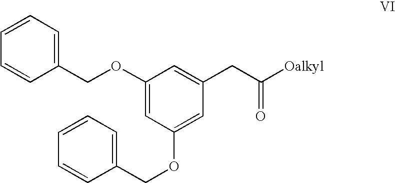

Intermediate VI can be easily prepared from reaction of 3,5-dihydroxyphenyl acetic acid methyl ester (commercially available) with benzyl bromide and potassium carbonate in DMF at room temperature.

Compounds of Formula I have a chiral center α to the carboxylic group, and can exist as one of two enantiomers (or a mixture thereof, wherein an enantiomeric excess may or may not be present). The enantiomers Ia (R enantiomer) and Ib (S enantiomer) are shown. The pure enantiomers Ia and Ib be obtained by chiral separation using a chiral column. The enantiomers Ia and Ib may also be separated by resolutions through forming chiral amine salts of the corresponding acids by fractional recrystallizations. The enantiomers Ia and Ib also may be obtained from kinectic resolution of the racemate of corresponding esters using lipase enzymes, such as Amano lipase Ak, Amano lipase PS, Amano lipase A, Amano lipase M, Amano lipase F-15, or Amano lipase G (from Biocatalytics Inc) in aqueous organic solvents, e.g. aqueous DMF, DMSO, t-butyl-ethyl ether or triton X-100 aqueous solutions.

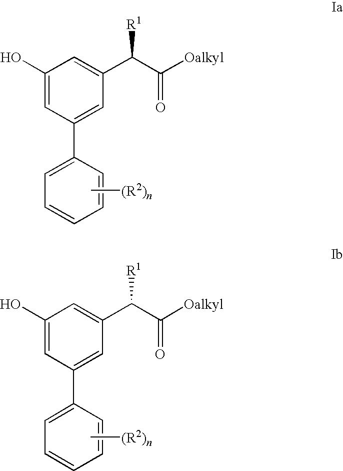

Both enantiomers of I may be prepared from chiral syntheses. Compounds of Formulas Ia and Ib may be obtained from esterification reactions following the removal of the chiral auxiliary groups from VIIa and VIIb respectively with lithium hydroxide in aqueous THF in the presence of hydrogen peroxide.

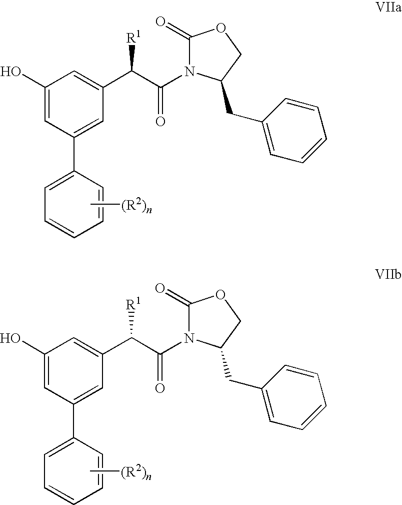

Compounds of Formulas VIIa and VIIb may be prepared from debenzylation of VIIIa and VIIIb respectively by hydrogenation in an alcohol solvent, e.g. MeOH or EtOH, in the presence of Pd—C.

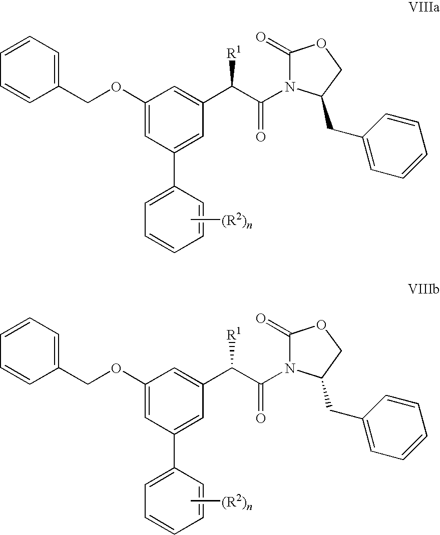

Compounds of Formulas VIIIa and VIIb may be prepared from the alkylation of IXa and IXb respectively with an appropriate alkyl bromide, including sec-butyl bromide or sec-butenyl bromide. Treatment of IXa and IXb in THF or other aprotic solvents with a base, such as lithium bis(trismethylsilyl)amide, sodium bis(trismethylsilyl)amide, or lithium diisopropylamide at −78° C., followed by the addition of an electrophile, for example sec-butyl bromide or sec-butenyl bromide gives alkylated compounds of Formulas VIIIa and VIIIb.

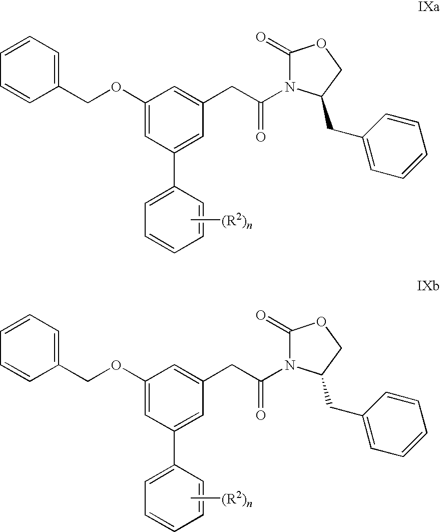

Compounds of Formulas IXa and IXb may be prepared from intermediate X by coupling with either R-isomer of 4-benzyl-oxazolidin-one XIa or S-isomer of 4-benzyl-oxazolidin-one XIb by Evans's procedures. Intermediate X may be reacted with pivaloyl chloride, oxalyl chloride or isopropyl chloroformate in THF in the presence of a base, e.g. triethylamine or N-methylmorpholine, to generate the mix anhydrides or acid chlorides which then are reacted with the lithium salt of XIa or XIb in THF. Other chiral auxillary groups may also be used in the chiral syntheses, e.g. chiral pseudoephedrine via the A. G. Myers conditions (J. Am. Chem. Soc. 1994, 116, 9361-9362). Treatment of either the carboxylic acid chloride or anhydride with an enantiomer of pseudoephedrine leads to amide derivative such as XIIa and XIIb. The amide is then treated with a strong base, e.g. lithium diisopropyl amide in the presence of lithium chloride, followed by the addition of an alkylating agent to yield the corresponding alkylated products XIIb and XIIc. The benzyl protecting group can be removed by hydrogenation or BBr₃ in DCM to give the chiral phenols XIIe and XIIf. The chiral auxillary group then can be removed in acid hydrolysis to give the homochiral targets Ia and Ib.

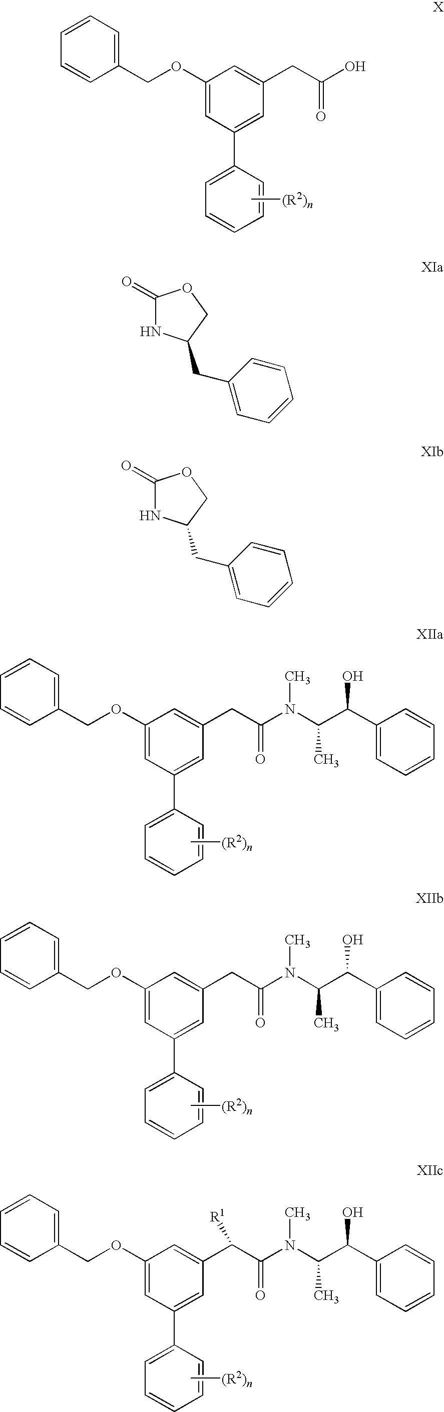

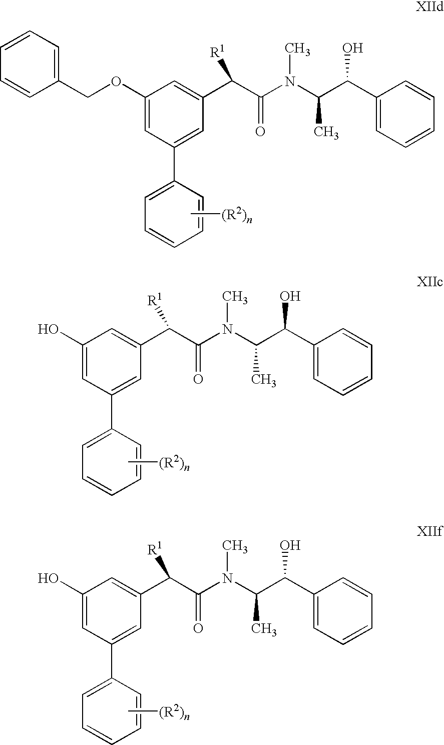

Intermediate X may be obtained from ester hydrolysis of III with base in aqueous alcohol solution, e.g. LiOH or NaOH in aqueous methanol solution.

### General Schemes Part II

Synthesis of γ-Secretase Modulators from Compounds of Formula I.

Scheme A illustrates the use of compounds of Formula I to generate γ-secretase modulators of Formula XIII. Alkylation of compounds of Formula I with benzyl bromides, benzyl chlorides, benzyl tosylates, or benzyl mesylates under typical benzylation conditions, e.g. in DMF or THF in the presence of base, such as potassium carbonate or cesium carbonate with temperature rages from 25-120° C. adds a benzyl group to I. Benzyl groups may also be added under Mitsnobu conditions, e.g. in THF or toluene in the presence of diethyl azodicarboxylate and triphenylphosphine. Ester hydrolysis of the benzylated intermediate under basic conditions yields compounds of Formula XIII.

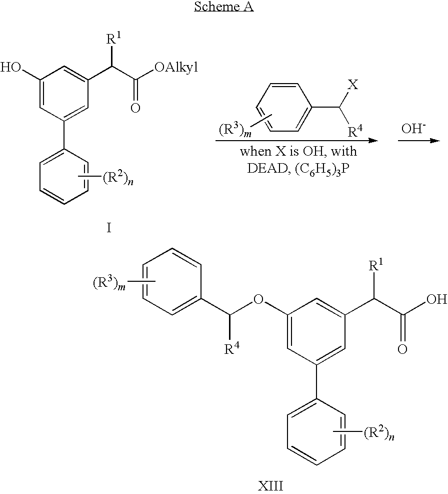

wherein X is Br, Cl, OMs, OH, OTos

Scheme B illustrates the use of compounds of Formula I to generate γ-secretase modulators of Formula XIV. Alcohol I is converted to the triflate by addition of triflic anhydride in pyridine and DCM at 0° C. and allowed to warm to room temperature; the triflate may alternatively be produced with N-phenyl-bis-(trifluoromethanesulfonimide) in THF in the presence of an amine base such as Et₃N at reflux. The resulting triflate is coupled with an aryl amine under typical Buckwald or Hartwig conditions, e.g. in toluene, dioxane or THF in the presence of potassium t-butoxide and a catalyst, e.g. palladium (II) acetate [Pd(OAc)₂] or Palladium (0) trans, trans-dibenzylideneacetone at elevated temperature (range from 80-180° C.) or the reaction may be performed in a microwave reactor to give the coupled product. The resulting aniline can be hydrolyzed under basic conditions to give compounds of Formula XIV, where R⁴ is H. Alternatively, said aniline may be alkylated with an alkyl halide, tosylate or mesylate and followed by hydrolysis to give compound XIV, where R⁴ is alkyl.

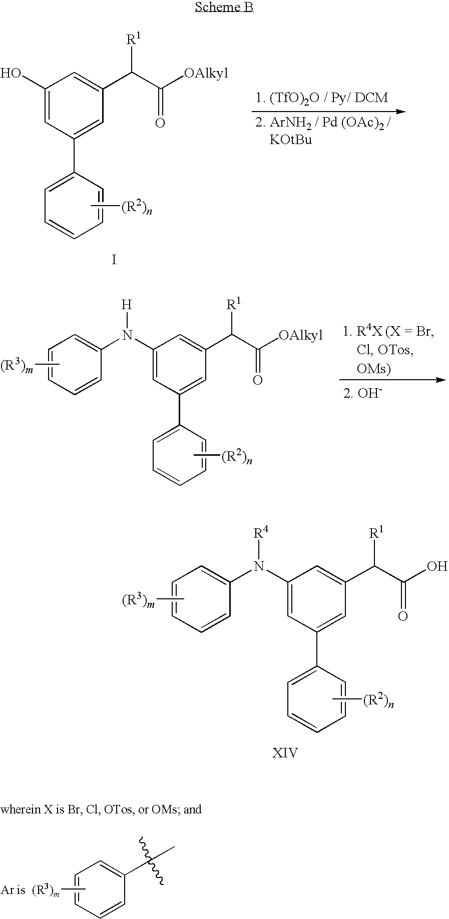

Scheme C illustrates the use of compounds of Formula I to generate γ-secretase modulators of Formula XV. Coupling compounds of Formula I with an aryl boronic acids in dicholoromethane (DCM) in the presence of a base, such as dimethylaminopyridine (DMAP) or triethylamine, molecular sieves, and Cu(OAc)₂ at room temperature gives a bi-arylether as described by D. Evans, et. al. (Tetrahedron Lettters (1980, 39(19), 2937-2940). Various additional reaction conditions for diaryl ether synthesis have been described in a review article by Rok Frlan and Danijel Kikkelj (Synthesis 2006, No 14, pp 2271-2285). Alternatively, compounds of Formula I can be converted to the triflate by reaction with triflic anhydride in DCM in the presence of pyridine; the triflate can then be coupled with a phenol to give a bi-arylether. All aforementioned bi-arylether intermediates can then be hydrolyzed under basic conditions to form γ-secretase modulators of Formula XV.

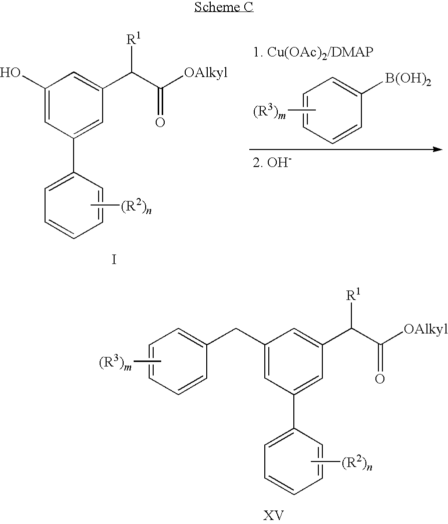

Scheme D illustrates the use of compounds of Formula I to generate γ-secretase modulators of Formula XVI. Compounds of Formula I can be converted to the triflate with triflic anhydride in pyridine and DCM or with N-phenyl-bis-(trifluoromethanesulfonimide) in THF in the presence of amine base such as Et₃N at reflux. The resulting triflate can be coupled with an aryl boronic acid under typical Suzuki coupling conditions, e.g. in DME, dioxane or THF in the presence of aqueous sodium carbonate solution and catalyst, e.g. tetrakis(triphenylphosphine)palladium(0) at a temperature range from 60-180° C. The reaction also can be carried out in a microwave reactor. The ester functionality of the resulting bi-phenyl intermediate may then be hydrolysed under basic conditions to give γ-secretase modulators of Formula XVI.

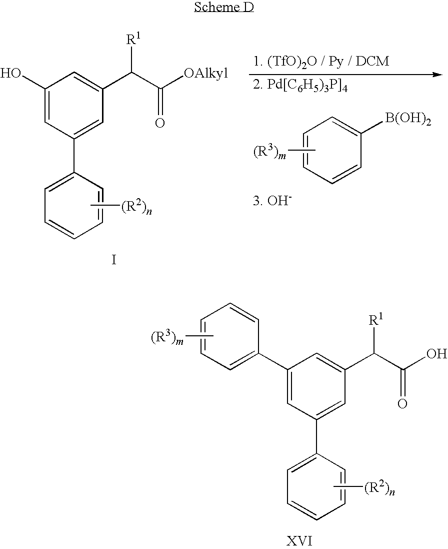

Scheme E illustrates the use of compounds of Formula I to generate γ-secretase modulators of Formulas XVII and XVIII. Compounds of Formula I can be converted to the triflate with triflic anhydride in pyridine and DCM or with N-phenyl-bis-(trifluoromethanesulfonimide) in THF in the presence of amine base such as Et₃N at reflux. The resulting triflate is treated with a heteroaryl boronic acid, including pyridyl boronic acid, under typical Suzuki coupling conditions, e.g. in DME, dioxane or THF in the presence of aqueous sodium carbonate solution and catalyst, e.g. tetrakis(triphenylphosphine)palladium(0) at a temperature range from 60-180° C. The Suzuki reaction can also be carried out in a microwave reactor. The coupled heteroaryl-phenyl ester is then hydrolyzed under basic conditions such as sodium hydroxide in aqueous alcohol solution to give γ-secretase modulators of Formula XVII. Compounds of Formula XVIII can be prepared from reduction of the heteroaryl (including pyridine) ring of the ester of XVII by hydrogenation with catalytic platinum oxide in an acidic alcohol medium such as methanol or ethanol. In cases where the resulting heterocycle contains N, compounds of Formula XVIII may be further derivatized by reductive alkylation with an aldehyde or alkylation with an alkyl halide or mesylate.

Alternatively, compounds of Formula XVIII can also be prepared from the alkylation the aforementioned pyridine-phenyl coupled Suzuki product with an alkyl halide or alkyl mesylate, followed by hydrogenation, and then basic hydrolysis as described above.

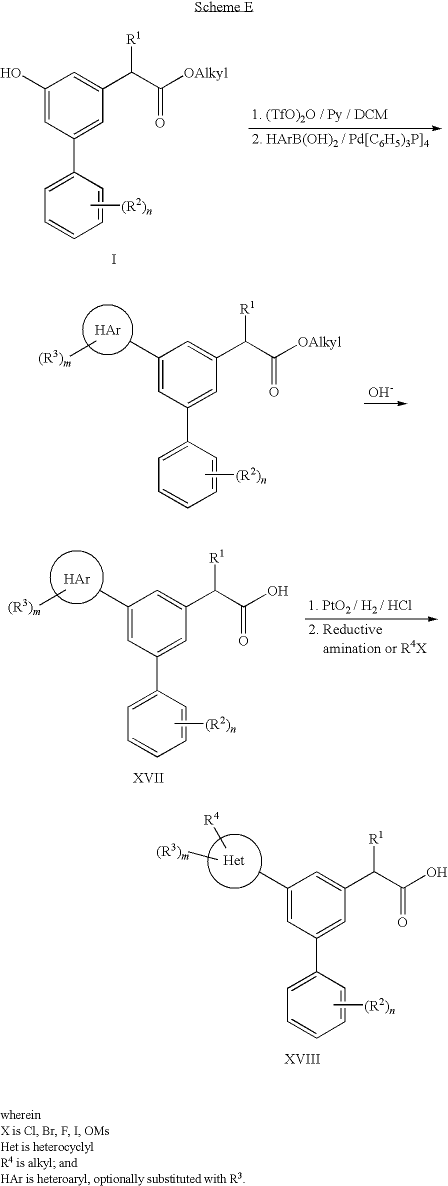

Scheme F illustrates the use of compounds of Formula I to generate γ-secretase modulators of Formulas XIX and XX. Compounds of Formula I can be converted to the triflate with triflic anhydride in pyridine and DCM or with N-phenyl-bis-(trifluoromethanesulfonimide) in THF in the presence of amine base such as Et₃N at reflux. The resulting triflate is then coupled with an arylboronic acid in the presence of potassium carbonate and potassium iodide with a catalytic amount of Pd(dppf)₂Cl₂ under a carbon monodioxide atmosphere. Hydrolysis under basic conditions gives compounds of Formula XIX. Alternatively, ester hydrolysis can be proceeded by ketone reduction using reagents such as sodium borohydride in order to give γ-secretase modulators of Formula XX.

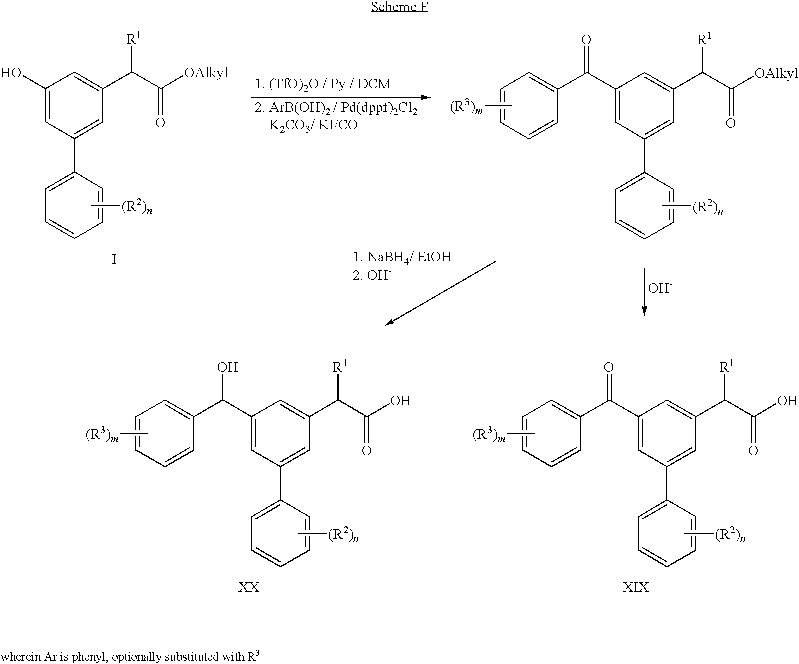

Scheme G illustrates the use of compounds of Formula I to generate γ-secretase modulators of Formula XXI. Compounds of Formula I can be converted to the triflate with triflic anhydride in pyridine and DCM or with N-phenyl-bis-(trifluoromethanesulfonimide) in THF in the presence of amine base such as Et₃N at reflux. The resulting triflate is then coupled with benzamide (optionally substituted with R³) in toluene under Buchwald conditions; in the presence of 2-(di-t-butylphosphino) 1,1′-binaphtahthyl and sodium-t-butoxide and a catalytic amount of Pd(OAc)₂ at elevated temperature (80-160° C.). The resulting intermediate is optionally alkylated using an alkyl halide, prior to ester hydrolysis yielding γ-secretase modulators of Formula XXI.

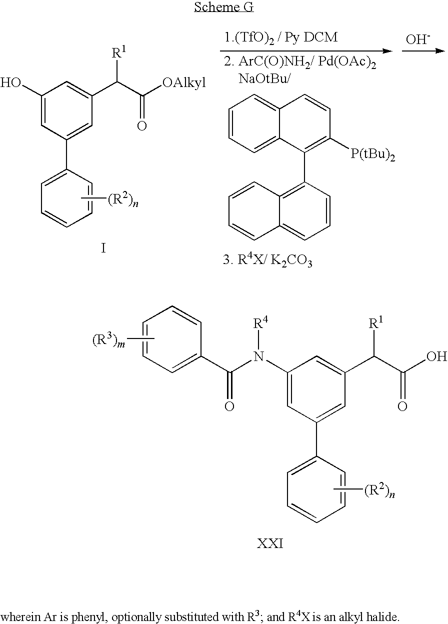

Scheme H illustrates the use of compounds of Formula I to generate γ-secretase modulators of Formulas XXII and XXIII. Compounds of Formula I can be converted to the triflate with triflic anhydride in pyridine and DCM or with N-phenyl-bis-(trifluoromethanesulfonimide) in THF in the presence of amine base such as Et₃N at reflux. The resulting triflate can then be coupled with an arylvinylboronic acid (optionally substituted with R³) under Suzuki coupling conditions. The resulting ester intermediate can be hydrolysed under basic conditions to yield γ-secretase modulators of Formula XXII. Alternatively, the same intermediate can be reduced by hydrogenation over catalytic Pd/C, followed by base mediated hydrolysis to give γ-secretase modulators of Formula XXIII.

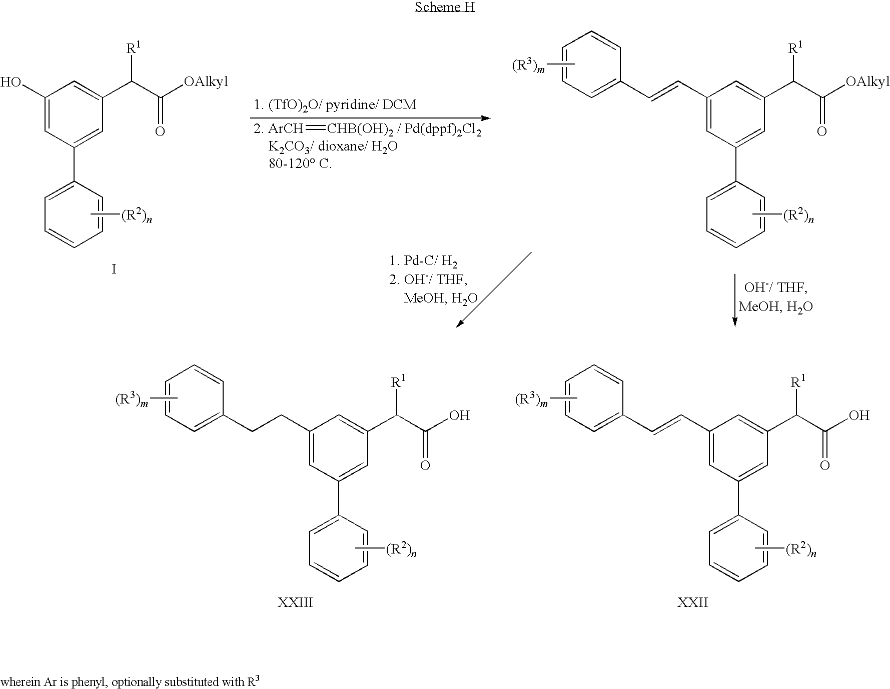

Scheme I illustrates the use of compounds of Formula I to generate γ-secretase modulators of Formula XXIV. Compounds of Formula I can be converted to the triflate with triflic anhydride in pyridine and DCM or with N-phenyl-bis-(trifluoromethanesulfonimide) in THF in the presence of amine base such as Et₃N at reflux. The resulting triflate can be reacted with zinc cyanide in an aprotic polar solvent, such as THF or DMF in the presence of triphenyl phosphine and a catalytic amount of tetrakis(triphenylphosphine) palladium (0) to give the corresponding cyano compound, which in turn can be reduced to the amine by hydrogenation with platinum oxide and hydrogen in an alcohol solvent. Alkylation of the resulting amine by reductive alkylation using an alkyl aldehyde and sodium triacetoxy borohydride, or sodium borohydride, and/or reaction with alkyl halides in DMF with potassium carbonate can install one or more alkyl groups on the amine functionality. Subsequent hydrolysis under basic conditions, such as NaOH or LiOH in THF/methanol/H₂O, yields γ-secretase modulators of Formula XXIV.

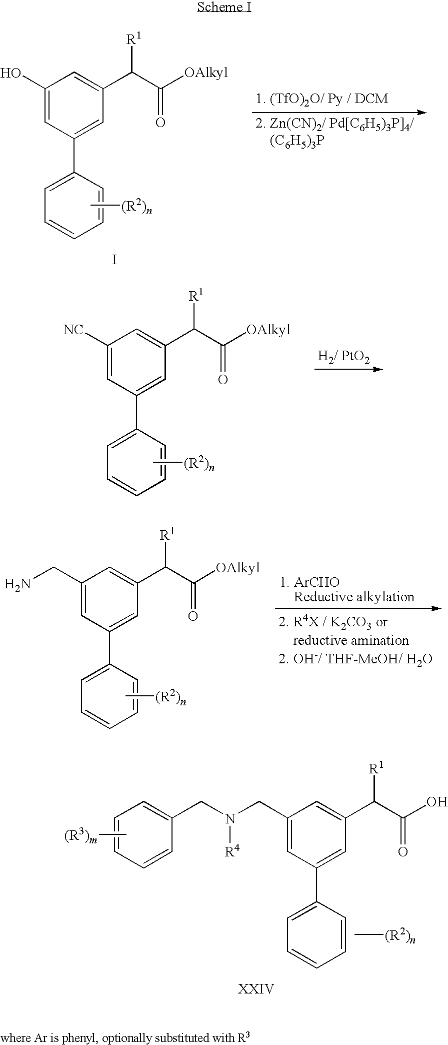

Scheme J illustrates the use of compounds of Formula I to generate γ-secretase modulators of Formula XXV. Compounds of Formula I can be converted to the triflate with triflic anhydride in pyridine and DCM or with N-phenyl-bis-(trifluoromethanesulfonimide) in THF in the presence of amine base such as Et₃N at reflux. The resulting triflate is then coupled with an amine in toluene under Buchwald conditions, such as potassium t-butoxide and catalytic Pd(OAc)₂ at 80-160° C., followed by basic hydrolysis to yield γ-secretase modulators of Formula XXV.

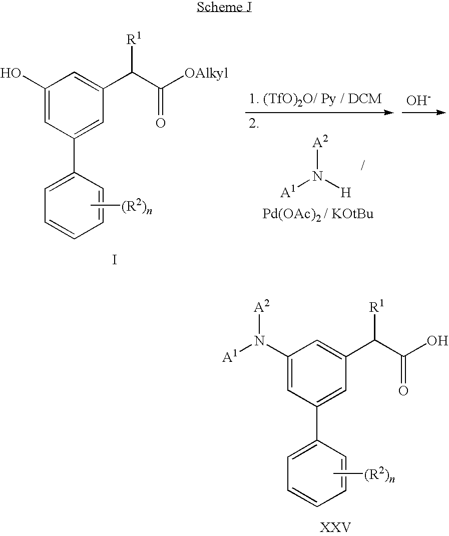

Scheme K illustrates the use of compounds of Formula I to generate γ-secretase modulators of Formulas XXVI and XXVII. Compounds of Formula I can be converted to the triflate with triflic anhydride in pyridine and DCM or with N-phenyl-bis-(trifluoromethanesulfonimide) in THF in the presence of amine base such as Et₃N at reflux. The resulting triflate is then reacted with diphenylketone imine in the presence of triphenylphosphine and a catalytic amount tetrakis(triphenylphosphine) palladium (0) at 50-160° C., followed by hydrolysis of the imine to obtain the amine. The resulting amino compound can then be functionalized via reductive amination with an aryl ketone or aryl aldehyde using sodium borohydride or triacetoxyboronhydride. Compounds of Formula XXVI can be generated by base mediated hydrolysis, or further alkylation of the amine by means known in the art, such as reaction with an alkyl halide or reductive alkylation with alkyl aldehyde or ketone, can precede the aforementioned hydrolysis to generate γ-secretase modulators of Formula XXVII.

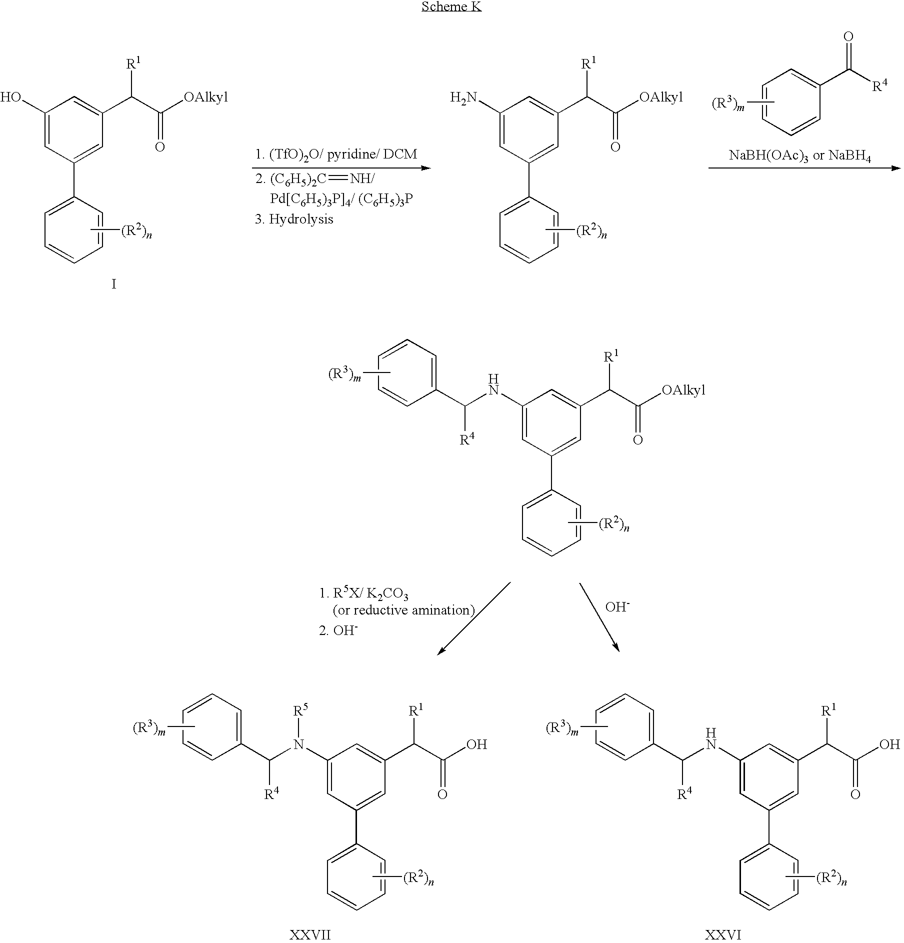

### Screening of the Compounds of the Invention for γ-Secretase-Modulating Activity

Screening was carried out using SKNBE2 cells carrying the APP 695-wild type, grown in DMEM/NUT-mix F12 (HAM) provided by Gibco (cat no. 31330-38) containing 5% Serum/Fe supplemented with 1% non-essential amino acids.

Cells were grown to near confluency.

The screening was performed using the assay as described in Citron et al (1997) Nature Medicine 3: 67.

Examples of the γ-secretase modulating activity of representative products of the invention are shown in the following table.

| | General | | | EC 50 |
|---|---|---|---|---|
| | Scheme | Structure | Chemical Name | WTAPP |
| 1 | A | 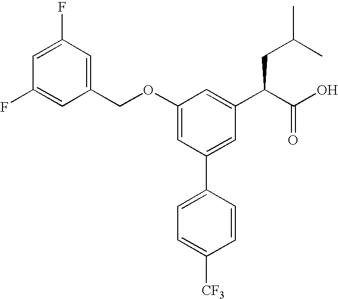 | (R) 2-[5-(3,5-Difluoro- benzyloxy)-4′- trifluoromethyl-biphenyl- 3-yl]-4-methyl-pentanoic acid | 0.53 |
| 2 | A | 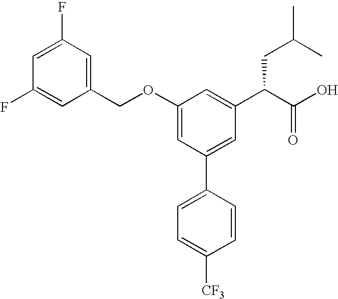 | (S) 2-[5-(3,5-Difluoro- benzyloxy)-4′- trifluoromethyl-biphenyl- 3-yl]-4-methyl-pentanoic acid | 1.52 |
| 3 | A | 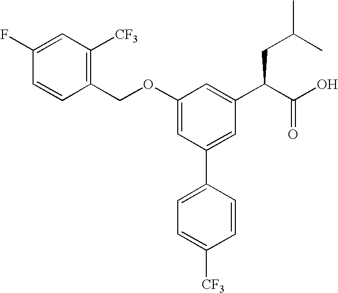 | (R)-2-[5-(4-fluoro-2- trifluoromethyl- benzyloxy)-4′- trifluoromethyl-biphenyl- 3-yl]-4-methyl-pentanoic acid | 0.57 |
| 4 | A | 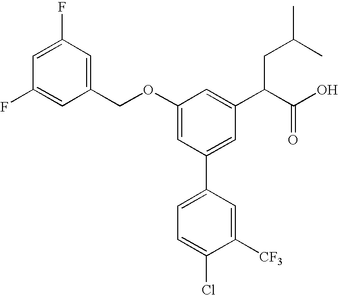 | 2-[4′-Chloro-5-(3,5- difluoro-2-benzyloxy)-3′- trifluoromethyl-biphenyl- 3-yl]-4-methyl-pentanoic acid | 0.31 |
| 5 | D | 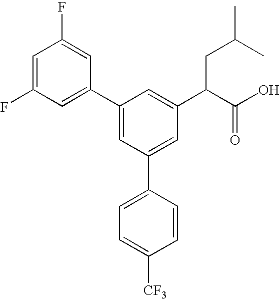 | 2-(3,5-Difluoro-4″- trifluoromethyl- [1,1′;3′,1″]terphenyl-5′-yl)- 4-methyl-pentanoic acid | 0.19 |
| 6 | D | 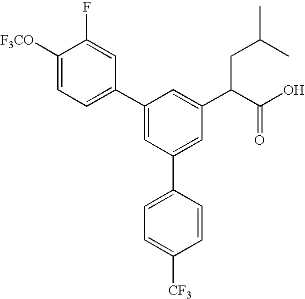 | 2-(3-Fluoro-4- trifluoromethoxy-4′- trifluoromethyl- [1,1′;3′,1′]terphenyl-5′-yl)- 4-methyl-pentanoic acid | 0.14 |
| 7 | D | 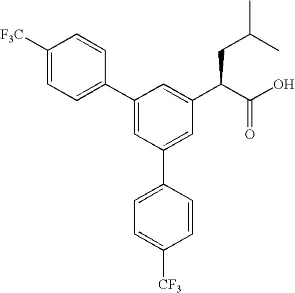 | (R)-2-(4,4″-Bis- trifluoromethyl- [1,1′;3′,1″]terphenyl-5′-yl)- 4-methyl-pentanoic acid | 0.22 |
| 8 | D | 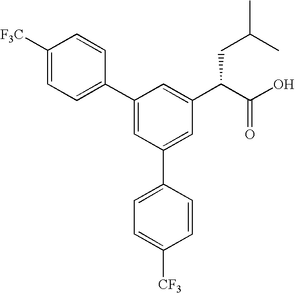 | (S)-2-(4,4″-Bis- trifluoromethyl- [1,1′;3′,1″]terphenyl-5′-yl)- 4-methyl-pentanoic acid | 0.25 |
| 9 | B | 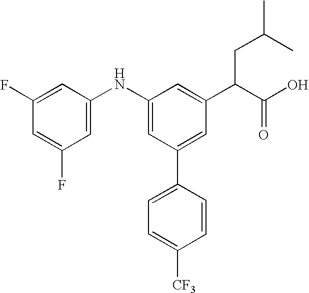 | 2-[5-(3,5-Difluoro- phenylamino)-4′- trifluoromethyl-biphenyl- 3-yl]-4-methyl-pentanoic acid | 73% inhibition @ 3 uM |
| 10 | B | 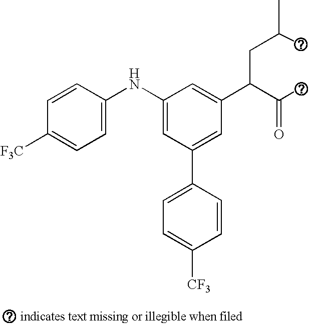 | 2-[5-(4-Fluoro-2- trifluoromethyl- phenylamino)-4′- trifluoromethyl-biphenyl- 3-yl]-4-methyl-pentanoic acid | 0.28 |
| 11 | B | 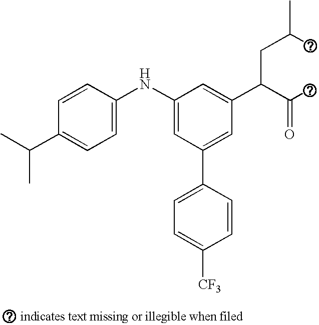 | 2-[5-(4-Isopropyl- phenylamino)-4′- trifluoromethyl-biphenyl- 3-yl]-4-methyl-pentanoic acid | 0.29 |
| 12 | B | 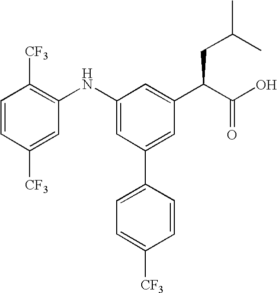 | (R)2-[5-(2,5-Bis- trifluoromethyl- phenylamino)-4′- trifluoromethyl-biphenyl- 3-yl]-4-methyl-pentanoic acid | 0.15 |
| 13 | B | 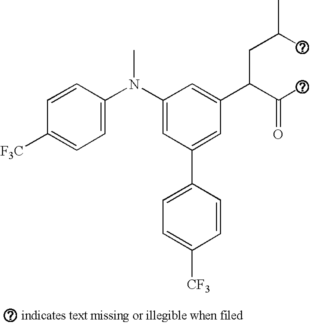 | 4-Methyl-2-{5-[methyl-(4- trifluoromethyl-phenyl)- amino]-4′-trifluoromethyl- biphenyl-3-yl}-pentanoic acid | 21% @ 0.3 uM |
| 14 | B | 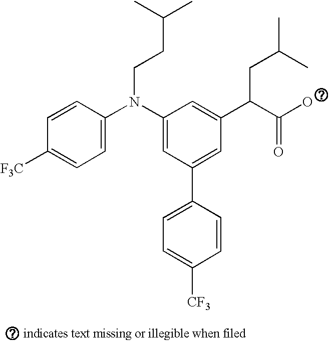 | 4-Methyl-2-{5-[(3-methyl- butyl)-(4-trifluoromethyl- phenyl)-amino]-4′- trifluoromethyl-biphenyl- 3-yl}-pentanoic acid | 0.17 |
| 15 | B | 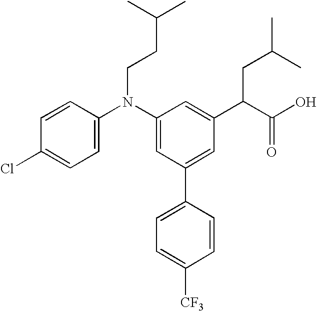 | 2-{5-[(4-Chloro-phenyl)- (3-methyl-butyl)-amino]- 4′-trifluoromethyl- biphenyl-3-yl}-4-methyl- pentanoic acid | 0.15 |
| 16 | C | 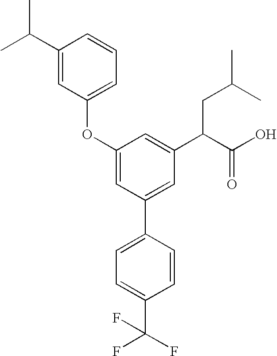 | 2-[5-(3-Isopropyl- phenoxy)-4′- trifluoromethyl-biphenyl- 3-yl]-4-methyl-pentanoic acid | 64% inhibition @ 3 uM |
| 17 | C | 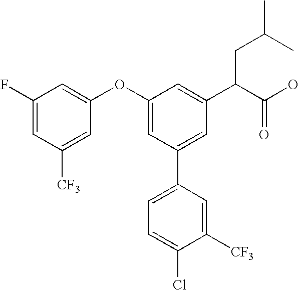 | 4-Methyl-2-[4′-chloro-3′- trifluoromethyl-5-(3- fluoro-5- trifluoromethylphenoxy)- biphenyl-3-yl]-pentanoic acid | 0.14 |
| 18 | C | 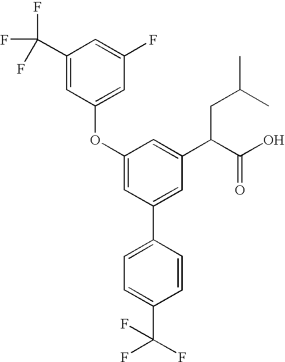 | 2-[5-(3-Fluoro-5- trifluoromethyl-phenoxy)- 4′-trifluoromethyl- biphenyl-3-yl]-4-methyl- pentanoic acid | 0.33 |
| 19 | E | 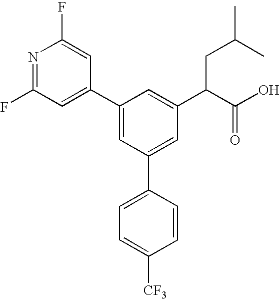 | 2-[5-(2,6-Difluoro-pyridin- 4-yl)-4′-trifluoromethyl- biphenyl-3-yl]-4-methyl- pentanoic acid | 0.22 |
| 20 | E | 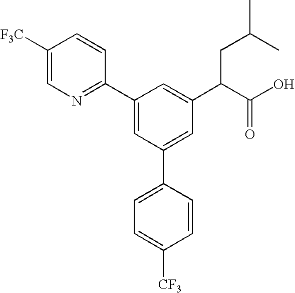 | 4-Methyl-2-[4′- trifluoromethyl-5-(5- trifluoromethyl-pyridin-2- yl)-biphenyl-3-yl]- pentanoic acid | 0.32 |
| 21 | A | 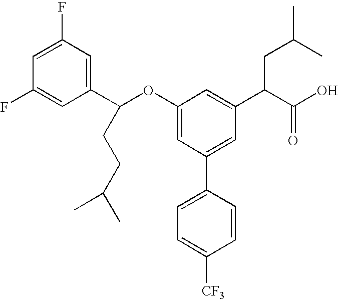 | 2-{5-[1-(3,5-Difluoro- phenyl)-4-methyl- pentyloxy]-4′-trifluoromethyl- biphenyl-3-yl}-4-methyl- pentanoic acid | 0.23 |
| 22 | F | 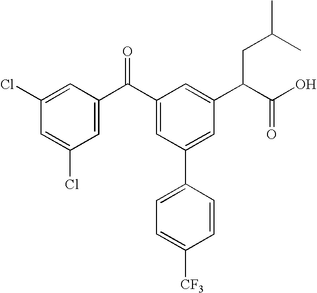 | 2-[5-(3,5-Dichloro- benzoyl)-4′-trifluoromethyl- biphenyl-3-yl]-4-methyl- pentanoic acid | 0.71 |
| 23 | H | 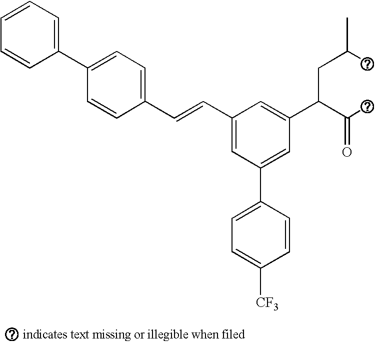 | 2-[5-(2-Biphenyl-4-yl- vinyl)-4′-trifluoromethyl- biphenyl-3-yl]-4-methyl- pentanoic acid | 0.14 |
| 24 | H | 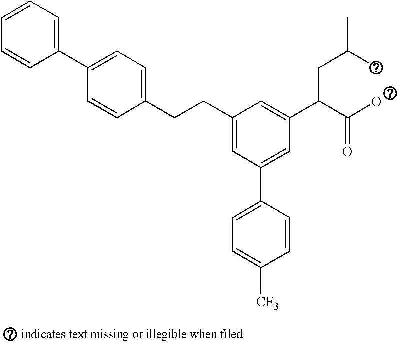 | 2-[5-(2-Biphenyl-4-yl- ethyl)-4′-trifluoromethyl- biphenyl-3-yl]-4-methyl- pentanoic acid | 0.21 |
| 25 | G | 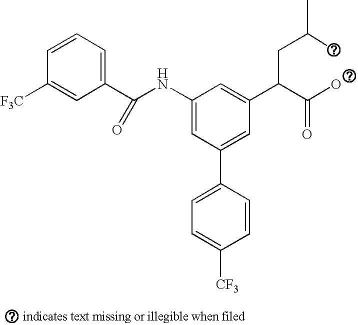 | 4-Methyl-2-[4′- trifluoromethyl-5-(3- trifluoromethyl- benzoylamino)-biphenyl-3- yl]-pentanoic acid | 1.79 |
| 26 | E | 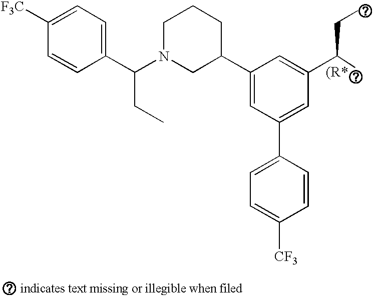 | (R*) 4-Methyl-2-(4′- trifluoromethyl-5-{1-[1-(4- trifluoromethyl-phenyl)- propyl]-piperidin-3-yl}- biphenyl-3-yl)-pentanoic acid | 0.36 |
| 27 | E | 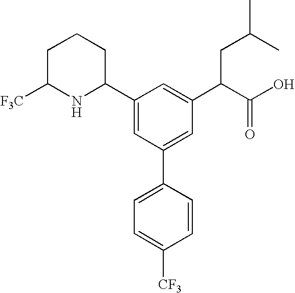 | 4-Methyl-2-[4′- trifluoromethyl-5-(6- trifluoromethyl-piperidin- 2-yl)-biphenyl-3-yl]- pentanoic acid | 0.7 |
| 28 | E | 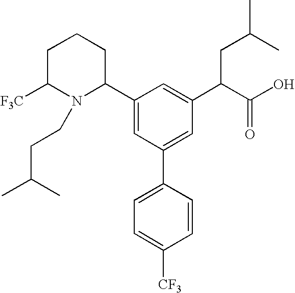 | 4-Methyl-2-{5-[1-(3- methyl-butyl)-6- trifluoromethyl-piperidin- 2-yl]-4′-trifluoromethyl- biphenyl-3-yl}-pentanoic acid | 0.11 |
| 29 | I | 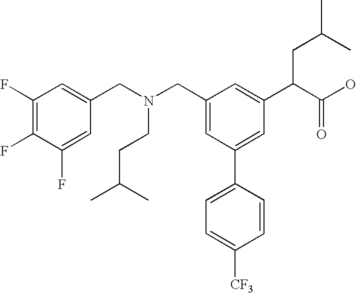 | 4-Methyl-2-(5-{[(3- methyl-butyl)-(3,4,5- trifluoro-benzyl)-amino]- methyl}-4′-trifluoromethyl- biphenyl-3-yl)-pentanoic acid | 0.27 |
| 30 | I | 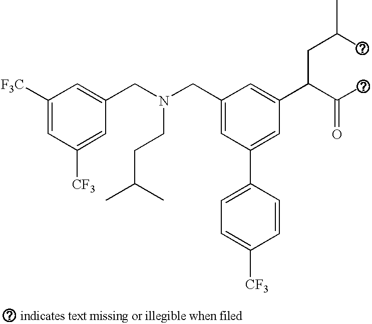 | 2-(5-{[(3,5-Bis- trifluoromethyl-benzyl)-(3- methyl-butyl)-amino]- methyl}-4′-trifluoromethyl- biphenyl-3-yl)-4-methyl- pentanoic acid | 0.25 |
| 31 | E | 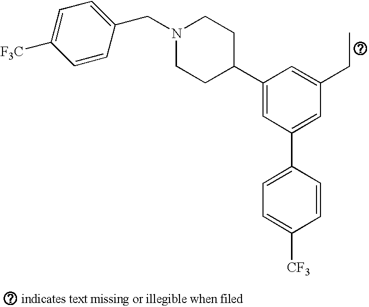 | 4-Methyl-2-{4′- trifluoromethyl-5-[1-(4- trifluoromethyl-benzyl)- piperidin-4-yl]-biphenyl-3- yl}-pentanoic acid | 8% @ 0.3 uM |
| 32 | E | 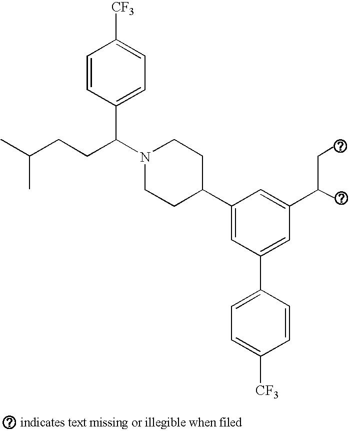 | 4-Methyl-2-(5-{1-[4- methyl-1-(4- trifluoromethyl-phenyl)- pentyl]-piperidin-4-yl}-4′- trifluoromethyl-biphenyl- 3-yl)-pentanoic acid | 2.08 |
| 33 | J | 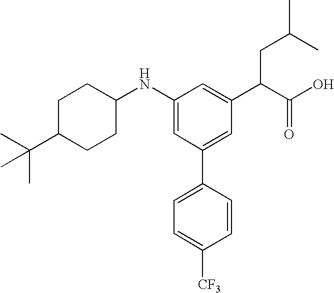 | 2-[5-(4-tert-Butyl- cyclohexylamino)-4′- trifluoromethyl-biphenyl- 3-yl]-4-methyl-pentanoic acid | 0.064 |
| 34 | J | 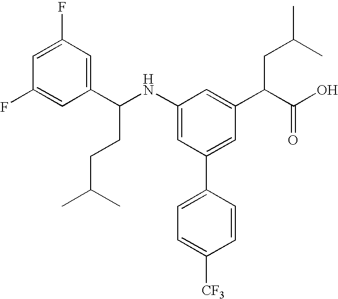 | 2-{5-[1-(3,5-Difluoro- phenyl)-4-methyl- pentylamino]-4′- trifluoromethyl-biphenyl- 3-yl}-4-methyl-pentanoic acid | 0.33 |
| 35 | E | 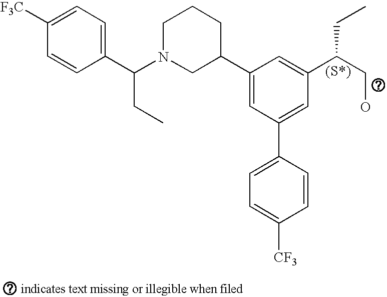 | (S*) 4-Methyl-2-(4′- trifluoromethyl-5-{1-[1-(4- trifluoromethyl-phenyl)- propyl]-piperidin-3-yl}- biphenyl-3-yl)-pentanoic acid | 0.59 |
| 36 | E | 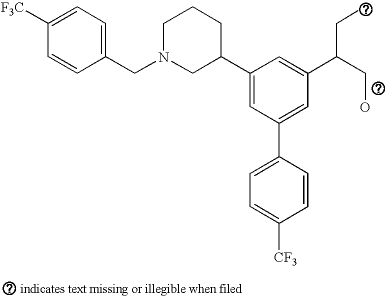 | 4-Methyl-2-{4′- trifluoromethyl-5-[1-(4- trifluoromethyl-benzyl)- piperidin-3-yl]-biphenyl-3- yl}-pentanoic acid | 0.51 |
| 37 | E | 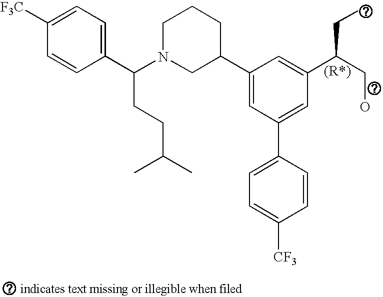 | (R*) 4-Methyl-2-(5-{1-[4- methyl-1-(4- trifluoromethyl-phenyl)- pentyl]-piperidin-3-yl}-4′- trifluoromethyl-biphenyl- 3-yl)-pentanoic acid | 44% @ 0.3 uM |
| 38 | E | 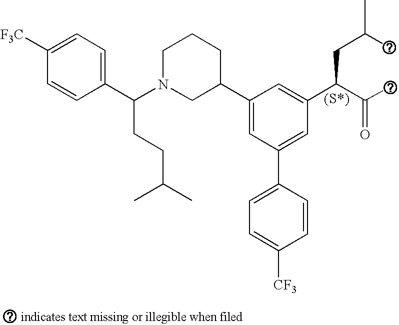 | (S*) 4-Methyl-2-(5-{1-[4- methyl-1-(4- trifluoromethyl-phenyl)- pentyl]-piperidin-3-yl}-4′- trifluoromethyl-biphenyl- 3-yl)-pentanoic acid | 0.45 |
| 39 | J | 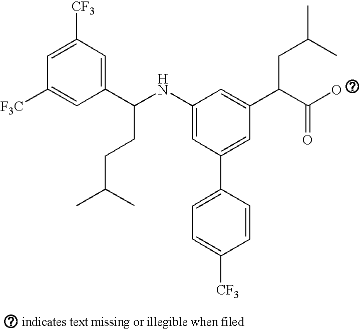 | 2-{5-[1-(3,5-Bis- trifluoromethyl-phenyl)-4- methyl-pentylamino]-4′- trifluoromethyl-biphenyl- 3-yl}-4-methyl- pentanoic acid | 0.22 |
| 40 | K | 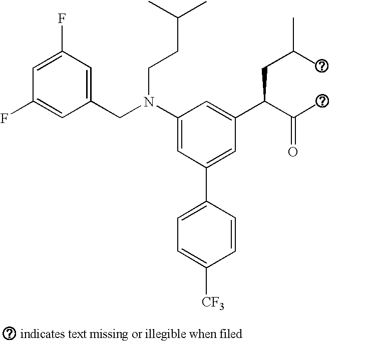 | 2-{5-[(3,5-Difluoro- benzyl)-(3-methyl-butyl)- amino]-4′-trifluoromethyl- biphenyl-3-yl}-4-methyl- pentanoic acid | 0.19 |

### Synthetic Procedures

All reactions were carried out under inert atmosphere unless otherwise stated. NMR spectra were obtained on a Bruker dpx400. LCMS was carried out on an Agilent 1100 using a ZORBAX® SB-C18, 4.6×75 mm, 3.5 micron column for method A. Column flow was 1 ml/min and solvents used were water and acetonitrile (0.1% TFA) with an injection volume of 10 ul. Wavelengths were 254 and 210 nm. The chiral purity analyses were performed by chiral columns

### Abbreviations

| | |
|---|---|
| Ac | Acetyl |
| tBu | tert-butyl |
| d | Doublet |
| dppf | 1,1′-bis(diphenylphosphino)ferrocene |
| DCM | Dichloromethane |
| DEAD | Diethylazodicarboxylate |
| DMAP | Dimethylaminopyridine |
| DME | 1,2-dimethoxyethane |
| DMF | N,N-dimethylformamide |
| DMSO | Dimethyl sulfoxide |
| Et | Ethyl |
| EtOAc | ethyl acetate |
| g | Gram |
| h | Hour |
| ISCO | Telydyne ISCO Chromatography |
| HPLC | high pressure liquid chromatography |
| K₂CO₃ | Potassium carbonate |
| l | Litre |
| LCMS | liquid chromatography-mass spectrometry |
| LDA | lithium diisopropylamide |
| M | Molar |
| m | Multiplet |
| Me | Methyl |
| min | Minute |
| mol | Mole |
| NMR | nuclear magnetic resonance |
| OMs | Mesylate |
| OTf | Triflate |
| OTs | Tosylate |
| Py | Pyridine |
| q | Quartet |
| RT or rt | Room temperature |
| s | Singlet |
| sat | Saturated |
| t | Triplet |
| TFA | Trifluoroacetic acid |
| THF | Tetrahydrofuran |

### EXAMPLES

### Example 1

### (R)2-[5-(3,5-Difluoro-benzyloxy)-4′-trifluoromethyl-biphenyl-3-yl]-4-methyl-pentanoic Acid

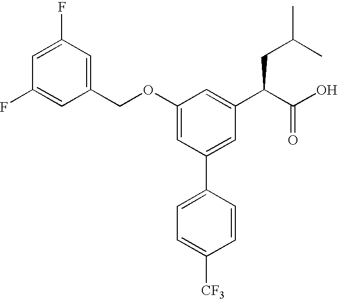

### Racemic Synthesis and Chiral Separation

### a) (3,5-Bis-benzyloxy-phenyl)-acetic Acid Methyl Ester

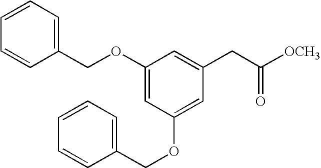

A mixture of (3,5-dihydroxy-phenyl)-acetic acid methyl ester (from Aldrich, 70 g, 0.385 mol), benzylbromide (137 mL, 1.16 mol), potassium carbonate (160 g, 1.16 mol) and DMF (1.5 L) under N₂ was mechanically stirred at room temperature overnight. The resulting reaction mixture was poured into a mixture of 1.5 L of ice-water with stirring. The precipitate was obtained by filtration and washed with heptane successively to remove benzyl bromide to give the title compounds (123.7 g) as a brown solid which was air dried for the next reaction. ¹H-NMR (CDCl₃): δ 3.60 (s, 2H), 3.71 (s, 3H), 5.05 (s, 4H), 6.60 (s, 3H), 7.35-7.50 (m, 10H); Calcd for C23H22O4 (M+H) 363.15, Found 363.

### b) 3-Benzyloxy-5-hydroxy-phenyl)-acetic Acid Ethyl Ester

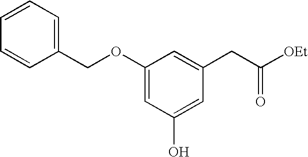

A solution of 3,5-Bis-benzyloxy-phenyl)-acetic acid methyl ester from the previous step (50 g, 1.38 mol) and NaOH (6.6 g, 1.65 mole) in 1 L of EtOH in the presence of 10% of Pd—C was hydrogenated in a Parr shaker until one equivalent of hydrogen was consumed. The mixture was acidified with concentrated HCl and then the catalyst and solvent were removed to give an oil residue. The crude product was purified by ISCO silica gel column chromatography (ISCO) using EtOAC-heptane as eluents (gradient from 10% to 75% of EtOAc) to give 25 g of (65% yield) the title compound ¹H-NMR (CDCl₃): δ 1.15-1.20 (t, 3H), 3.4-(s, 2H), 4.05-4.1 (q, 2H), 4.9 (s, 2H), 5.5 (s, 1H), 6.4 (s, 2H), 6.5 (s, 1H), 7.207.35 (m, 5H); Calcd for C17H18O4 (M+H) 287.3, Found 287.

### c) (3-Benzyloxy-5-trifluoromethanesulfonyloxy-phenyl)-acetic Acid Ethyl Ester

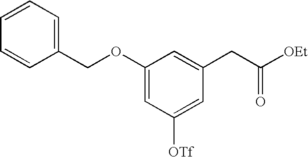

To a solution of 3-(benzyloxy-5-hydroxy-phenyl)-acetic acid ethyl ester from the previous step (74.4 g, 0.26 mol) in dichloromethane (700 mL) was added pyridine (62.5 mL, 0.78 mol). The mixture was cooled to 0° C. To this cold solution was added trifluoromethanesulfonic anhydride (65.6 mL, 0.39 mol), over 1.5 h, maintaining the internal temperature below 5° C. and stirred for an additional 0.5 h at 0° C. This reaction mixture was poured to a mixture of 1 N HCl (420 mL), and ice (105 g) and stirred for 0.5 h. The aqueous layer was extracted with dichloromethane (2×100 mL). Combined fractions were washed with water (2×100 mL), saturated aqueous NaHCO₃ solution (2×100 mL), and brine (2×100 mL). The organics were dried (MgSO₄) and concentrated in vacuo to receive a reddish liquid (108 g) which was carried on to the next step without further purification.

Calcd for C18H17F3O6S (M+H) 419.07, Found 419.1.

### d) (5-Benzyloxy-4′-trifluoromethyl-biphenyl-3-yl)-acetic Acid Ethyl Ester

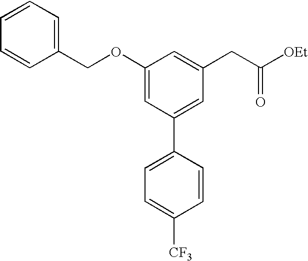

A mixture of (3-benzyloxy-5-trifluoromethanesulfonyloxy-phenyl)-acetic acid ethyl ester from the previous step (108 g, 0.26 mol), 4-(trifluoromethyl)phenylboronic acid (55.6 g, 0.29 mol), 1,2-dimethoxyethane (1.1 L) and aqueous Na₂CO₃ (2 M, 129 mL, 0.26 mol) was mechanically stirred while purging with N₂ at room temperature for 10 min. To this system was added Pd(Ph₃)₄ (480 mg, 0.42 mmol) and heated to reflux (95° C.) for 2.5 h. The red-brown mixture was diluted with EtOAc (0.5 L) and washed with saturated aqueous NaHCO₃ solution (3×200 mL) and brine (2×200 mL). The organic fraction was dried (Na₂SO₄) and concentrated in vacuo. The crude mixture was purified by ISCO column chromatography to obtain (5-benzyloxy-4′-trifluoromethyl-biphenyl-3-yl)-acetic acid ethyl ester (107 g, 100%).

¹H-NMR (CDCl₃): δ 1.26 (t, 3H), 3.66 (s, 2H), 4.17 (q, 2H), 5.12 (s, 2H), 6.99 (s, 1H), 7.12 (s, 2H), 7.34-7.49 (m, 5H), 7.67 (s, 4H); Calcd for C24H21F3O3 (M+H) 415.14, Found 415.2.

### e) 2-(5-Benzyloxy-4′-trifluoromethyl-biphenyl-3-yl)-4-methyl-pent-4-enoic Acid Ethyl Ester

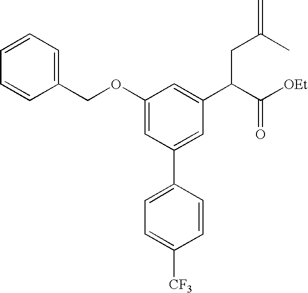

To a solution of compound 1d (4.9 g, 11.8 mmol) in THF (50 mL) at −78° C. was added Li[N(SiMe₃)₂] (1N in THF, 14.2 mL, 14.2 mmol) dropwise. The reaction mixture was stirred for 1 h at −78° C. and then 3-bromo-2-methyl-propene (1.25 mL, 12.4 mmol) was added dropwise. The solution was slowly warmed up to −35° C. and stirred at −35° C. for 0.5 h. The reaction was quenched with NH₄Cl saturated solution and extracted with EtOAc. The organic extracts was dried (Na₂SO₄), concentrated and purified by column chromatography give compound 1e (5.1 g, 92%) as a clear oil; ¹H NMR (400 MHz, CHLOROFORM-D) δ ppm 1.19-1.29 (m, 3H), 1.74 (s, 3H), 2.47 (m, 1H), 2.85 (m, 1H), 3.83 (m, 1H), 4.11 (m, 2H), 4.72 (s, 1H), 4.77 (s, 1H), 5.12 (s, 2H), 7.03 (s, 1H), 7.10 (s, 1H), 7.15 (s, 1H), 7.35-7.48 (m, 5H), 7.67 (s, 4H); Calcd for C28H27F3O3 (M+H) 469.19, Found 469.

### f) 2-(5-Hydroxy-4′-trifluoromethyl-biphenyl-3-yl)-4-methyl-pentanoic Acid Ethyl Ester

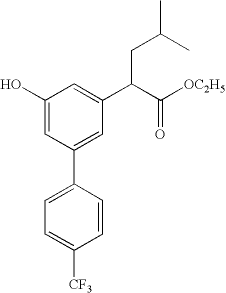

A mixture of compound 1e (5.1 g, 10.9 mmol), 10% Pd/C (500 mg) in EtOH (50 mL) was hydrogenated under H₂ (40 psi) in par-shaker for 20 h. The resulting reaction mixture was filtered through a celite pad and the filtrate was concentrated to give compound 1f (4.2 g, 100%) as a clear oil; 1H NMR (300 MHz, CHLOROFORM-D) δ ppm 0.92 (d, J=6.6 Hz, 6H), 1.25 (m, 3H), 1.49-1.61 (m, 1H), 1.65-1.70 (m, 1H), 1.95-2.05 (m, 1H), 3.67 (t, J=7.7 Hz, 1H), 4.10-4.29 (m, 2H), 6.91 (s, 1H), 6.97 (t, J=2.0 Hz, 1H), 7.08 (s, 1H), 7.65 (s, 4H); Calcd for C21H23F3O3 (M+H) 381.16, Found 381.

### g) 2-[5-(3,5-Difluoro-benzyloxy)-4′-trifluoromethyl-biphenyl-3-yl]-4-methyl-pentanoic Acid

To a solution of compound 1f (4 g, 10 mmol) in DMF was added cesium carbonate (4.89 g, 15 mmol) and then 3, 5 difluorobenzylbromide (3.18 g 15 mmol). The resulting solution was stirred at room temperature for 18 h and then was quenched with water. The aqueous solution was extracted with EtOAc. The organic layer was washed, dried and evaporated to give a residue (5 g). The crude was then in 1N KOH in MeOH (3 eq.) at rt overnight. The solution was acidified with con. HCl and then was extracted with EtOAc. The organic layer was then washed with water, dried over Na2SO4, then evaporated on a rotary evaporator to give a crude product. The crude was triturated heptane to afford 4.3 g (91% yield) of (R) and (S) product.

The enantiomers were resolved by chromatographic separation using a Chiralpak AD column using methanol and acetonitrile containing 0.1% of formic acid as an eluent to obtain (R) enantiomer, Compound 1, and (S) enantiomer, Compound 2, respectively. The (R) enantiomer was found to has rotation −27.29 degrees in MeOH and the (S) enantiomer has rotation +25.2 degrees in MeOH. The absolute stereochemistry centers were assigned by correlation with the synthetic materials described below.

### Chrial Synthesis of (R)-2-[5-(3,5-Difluoro-benzyloxy)-4′-trifluoromethyl-biphenyl-3-yl]-4-methyl-pentanoic Acid

### h) 5-Benzyloxy-4′-trifluoromethyl-biphenyl-3-yl)-acetic Acid

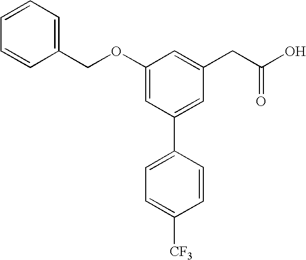

To a solution of (5-benzyloxy-4′-trifluoromethyl-biphenyl-3-yl)-acetic acid ethyl ester (example 1d, 120 g, 0.29 mol) in THF (1.2 L) was added water (240 mL), LiOH.H2O (16 g, 0.32 mol) and the resulting mixture was stirred at room temperature for 16 h. The solution was filtered and concentrated in vacuo to remove THF. The resulting thick liquid was acidified to pH 2 by adding 2N aqueous HCl solution and the white suspension was mechanically stirred for 1 h at room temperature. The wet white product was recovered after filtration and dissolved in EtOAc (500 mL). The organic layer was separated from water, dried (MgSO₄) and concentrated in vacuo to obtain (5-benzyloxy-4′-trifluoromethyl-biphenyl-3-yl)-acetic acid (105 g, 94%).

¹H-NMR (d₆-DMSO): δ 3.64 (s, 2H), 5.18 (s, 2H), 7.02 (s, 1H), 7.24 (d, 2H), 7.34-7.50 (m, 5H), 7.81 (d, 2H), 7.89 (d, 2H), 12.25 (bs, 0.6H); Calcd for C22H17F3O3 (M+H) 387.11, Found 387.1.

### i) 4-Benzyl-3-[2-(5-benzyloxy-4′-trifluoromethyl-biphenyl-3-yl)-acetyl]-oxazolidin-2-one

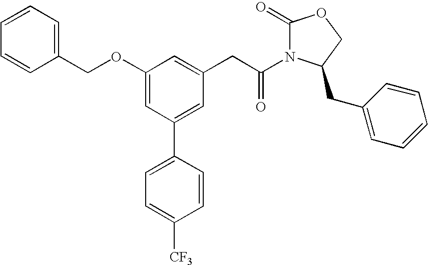

To a mechanically stirred solution of (5-benzyloxy-4′-trifluoromethyl-biphenyl-3-yl)-acetic acid from the previous step (20 g, 52 mmol) in THF (104 mL) at −78° C. was added N-methyl morpholine (NMM) (6.3 mL, 57 mmol) and trimethylacetyl chloride (7.0 mL, 57 mmol) maintaining the internal temperature below −70° C. This mixture was stirred at −78° C. for 15 minutes and at 0° C. for 1 h. The white solid was filtered off and the filtrate containing the mixed anhydride cooled back to −78° C. for the subsequent reaction. In a separate flask, to a solution of (R)-(+)-4-benzyl-2-oxazolidinone (9.6 g, 54.4 mmol) in THF (109 mL) at −78° C. was added nBuLi (1.6M in hexanes, 34 mL, 54.4 mol), drop-wise, maintaining the internal temperature below −70° C. and stirred for 45 min. This metalated chiral auxiliary was cannulated to add to a reaction flask containing the anhydride solution at −78° C. The reaction was stirred and allowed to warm to 0° C. over 1.5 h. The resulting mixture was stirred further at 0° C. for 30 minute and quenched by adding excess saturated aqueous NH₄Cl solution. The solution was diluted with EtOAc (200 mL) and the organic phase was washed with saturated aqueous NaHCO₃ solution (3×100 mL) and brine (2×100 mL). The solution was dried over MgSO₄ and the solvent was removed in vacuo. The crude material was purified by ISCO silica gel column chromatography to yield 20.3 g (72%) of 4-benzyl-3-[2-(5-benzyloxy-4′-trifluoromethyl-biphenyl-3-yl)-acetyl]-oxazolidin-2-one as a white solid.

¹H-NMR (CDCl₃): δ 2.76 (dd, 1H), 3.26 (dd, 1H), 4.19 (m, 2H), 4.35 (q, 2H), 4.69 (m, 1H), 5.13 (s, 2H), 7.04-7.46 (m, 13H), 7.67 (s, 4H); Calcd for C32H26F3NO4 (M+H) 546.18, Found 546.3.

### j) 4-Benzyl-3-[2-(5-benzyloxy-4′-trifluoromethyl-biphenyl-3-yl)-4-methyl-pent-4-enoyl]-oxazolidin-2-one

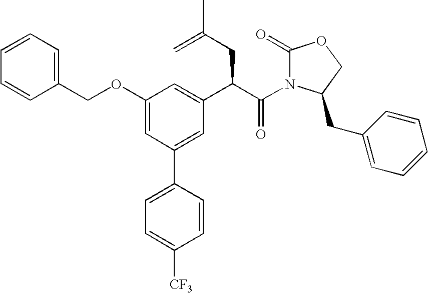

To a colorless solution of 4-benzyl-3-[2-(5-benzyloxy-4′-trifluoromethyl-biphenyl-3-yl)-acetyl]-oxazolidin-2-one from the previous step (6.0 g, 11.00 mmol) in dry THF (22 mL) at −78° C. was added sodium bis(trimethylsilyl)amide (NaHMDS) (1 M in THF solution, 12.11 mL, 12.11 mmol), drop-wise, maintaining the internal temperature below −75° C. The resulting red solution was stirred at −78° C. for 30 minutes. To this was added 3-bromo-2-methyl propene (4.44 mL, 44 mmol) maintaining the temperature below −75° C. When the addition was near completion, the reaction mixture turned green. At this point the dry-ice bath was quickly removed and replaced with water-ice bath, and the addition was completed. The reaction mixture was stirred at 0° C. for an additional 30 min and quenched with saturated aqueous NH₄Cl solution. The system was diluted with EtOAC (100 mL) and the organic phase was washed with saturated aqueous NaHCO₃ solution (3×50 mL) and dried (MgSO₄). Solvent was removed in vacuo and the crude mixture was purified by ISCO silica gel column to yield 4-benzyl-3-[2-(5-benzyloxy-4′-trifluoromethyl-biphenyl-3-yl)-4-methyl-pent-4-enoyl]-oxazolidin-2-one (6.3 g, 95%).

¹H-NMR (CDCl₃): δ 1.80 (s, 3H), 2.46 (dd, 1H), 2.75 (dd, 1H), 3.05 (dd, 1H), 3.32 (dd, 1H), 4.08 (m, 2H), 4.59 (m, 1H), 4.80 (d, 2H), 5.13 (s, 2H), 5.48 (dd, 1H), 7.11 (d, 2H), 7.21-7.49 (m, 11H), 7.67 (s, 4H); Calcd for C36H32F3NO4 (M+H) 600.23, Found 600.3.

### k) 4-Benzyl-3-[2-(5-hydroxy-4′-trifluoromethyl-biphenyl-3-yl)-4-methyl-pentanoyl]-oxazolidin-2-one

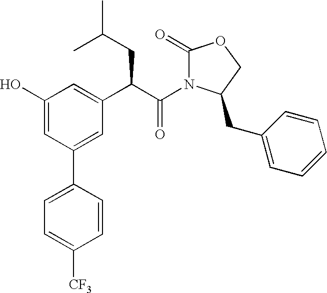

To a solution of 4-benzyl-3-[2-(5-benzyloxy-4′-trifluoromethyl-biphenyl-3-yl)-4-methyl-pent-4-enoyl]-oxazolidin-2-one from the previous step (6.7 g, 11.2 mmol) in MeOH (150 mL) was added 10% Pd/C (670 mg, 10 w %). The black suspension was hydrogenated at 5-50 psi overnight. The mixture was filtered through a celite pad and the solvent was removed in vacuo to obtain relatively pure 4-benzyl-3-[2-(5-hydroxy-4′-trifluoromethyl-biphenyl-3-yl)-4-methyl-pentanoyl]-oxazolidin-2-one (5.4 g, 93%).

¹H-NMR (CDCl₃): δ 0.94 (d, 3H), 0.98 (d, 3H), 1.54 (m, 1H), 1.74 (m, 1H), 2.12 (m, 1H), 2.79 (dd, 1H), 3.36 (dd, 1H), 4.11 (m, 2H), 4.62 (m, 1H), 5.25 (t, 1H), 6.97 (m, 2H), 7.21-7.37 (m, 6H), 7.67 (s, 4H); Calcd for C29H28F3NO4 (M+H) 512.20, Found 512.3.

### l) 4-Benzyl-3-{2-[5-(3,5-difluoro-benzyloxy)-4′-trifluoromethyl-biphenyl-3-yl]-4-methyl-pentanoyl}-oxazolidin-2-one

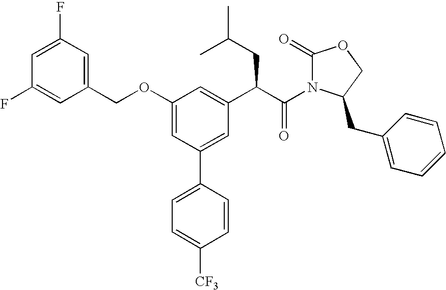

To a solution of 4-benzyl-3-[2-(5-hydroxy-4′-trifluoromethyl-biphenyl-3-yl)-4-methyl-pentanoyl]-oxazolidin-2-one from the previous step (18.77 g, 36.73 mmol) in acetonitrile (184 mL) at 0° C. was added 1-bromomethyl-3,5-difluoro-benzene (7.13 mL, 55.10 mmol) and Cs₂CO₃ (23.94 g, 73.46 mmol) in portions over 5 minutes. The resulting white suspension was stirred at room temperature for 2 h. The white solid was filtered off and the solvent was removed in vacuo to obtain relatively pure 4-benzyl-3-{2-[5-(3,5-difluoro-benzyloxy)-4′-trifluoromethyl-biphenyl-3-yl]-4-methyl-pentanoyl}-oxazolidin-2-one.

### m) 2-[5-(3,5-Difluoro-benzyloxy)-4′-trifluoromethyl-biphenyl-3-yl]-4-methyl-pentanoic Acid

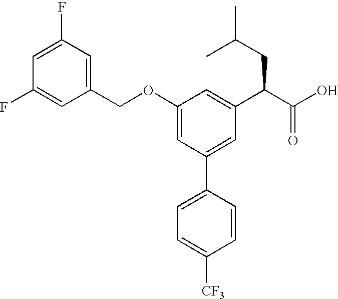

To a solution of 4-benzyl-3-{2-[5-(3,5-difluoro-benzyloxy)-4′-trifluoromethyl-biphenyl-3-yl]-4-methyl-pentanoyl}-oxazolidin-2-one (23.40 g, 36.73 mmol) in THF (180 mL) was added water (60 mL). The system was cooled to 0° C. To this cold solution was added LiOH.H2O (1.54 g, 36.73 mmol) and 30% H₂O₂ (16.65 mL, 146.92 mmol), drop-wise, maintaining the internal temperature below 5° C. The resulting cloudy solution was stirred at 0° C. for 20 min. The excess H₂O₂ was quenched by adding 1.5 M aqueous Na₂SO₃ solution (97.9 mL, 146.92 mmol) and stirred at room temperature for 15 min. The organic solvent was removed in vacuo. The resulting liquid was acidified to pH 2 by adding 1 N aqueous HCl solution. The aqueous layer was extracted with EtOAc (3×200 mL), dried over MgSO₄, and concentrated in vacuo resulting in a crude mixture which was purified by ISCO silica gel column chromatography to yield (R)-2-[5-(3,5-difluoro-benzyloxy)-4′-trifluoromethyl-biphenyl-3-yl]-4-methyl-pentanoic acid (12.25 g, 70%).

¹H-NMR (CDCl₃): δ 0.93 (d, 6H), 1.51 (m, 1H), 1.72 (m, 1H), 1.98 (m, 1H), 3.72 (t, 1H), 5.09 (s, 2H), 6.76 (m, 1H), 6.98 (m, 3H), 7.07 (t, 1H), 7.17 (s, 1H), 7.66 (m, 4H); Calcd for C26H23F5O3 (M+H) 479.45, Found 479.2.

### Example 2

### (S)-2-[5-(3,5-Difluoro-benzyloxy)-4′-trifluoromethyl-biphenyl-3-yl]-4-methyl-pentanoic Acid

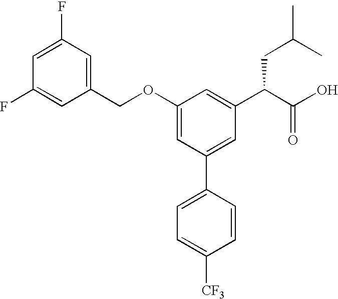

### a) 4-Benzyl-3-[2-(5-hydroxy-4′-trifluoromethyl-biphenyl-3-yl)-4-methyl-pentanoyl]-oxazolidin-2-one

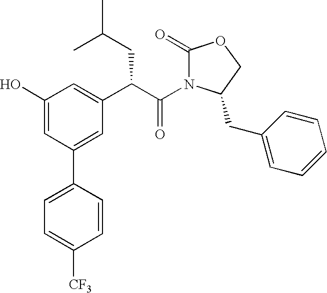

The title compound was prepared from 4-benzyl-3-[2-(5-benzyloxy-4′-trifluoromethyl-biphenyl-3-yl)-4-methyl-pent-4-enoyl]-oxazolidin-2-one following the same procedure as for the synthesis of Example 1, step (k).

¹H-NMR (CDCl₃): δ 0.94 (d, 3H), 0.98 (d, 3H), 1.54 (m, 1H), 1.74 (m, 1H), 2.12 (m, 1H), 2.79 (dd, 1H), 3.36 (dd, 1H), 4.11 (m, 2H), 4.62 (m, 1H), 5.25 (t, 1H), 6.97 (m, 2H), 7.21-7.37 (m, 6H), 7.67 (s, 4H); Calcd for C29H28F3NO4 (M+H) 512.20, Found 512.3.

### b) 4-Benzyl-3-{2-[5-(3,5-difluoro-benzyloxy)-4′-trifluoromethyl-biphenyl-3-yl]-4-methyl-pentanoyl}-oxazolidin-2-one

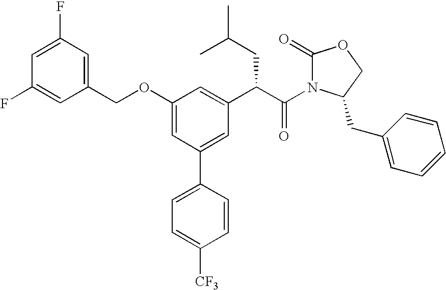

To a solution of 4-benzyl-3-[2-(5-hydroxy-4′-trifluoromethyl-biphenyl-3-yl)-4-methyl-pentanoyl]-oxazolidin-2-one from the previous step (0.40 g, 0.78 mmol) in acetonitrile (3 mL) at room temperature was added 1-bromomethyl-3,5-difluoro-benzene (0.243 g, 1.17 mmol) and Cs₂CO₃ (0.508 g, 1.56 mmol). The resulting white suspension was stirred for 1 h. The white solid was filtered off and the solvent was removed in vacuo to obtain relatively pure 4-benzyl-3-{2-[5-(3,5-difluoro-benzyloxy)-4′-trifluoromethyl-biphenyl-3-yl]-4-methyl-pentanoyl}-oxazolidin-2-one.

### c) 2-[5-(3,5-Difluoro-benzyloxy)-4′-trifluoromethyl-biphenyl-3-yl]-4-methyl-pentanoic Acid

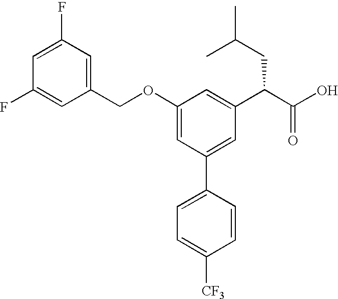

To a solution of 4-benzyl-3-{2-[5-(3,5-difluoro-benzyloxy)-4′-trifluoromethyl-biphenyl-3-yl]-4-methyl-pentanoyl}-oxazolidin-2-one from the previous step (0.425 g, 0.67 mmol) in THF (10 mL) was added water (3.5 mL). The system was cooled to 0° C. To this cold solution was added LiOH.H2O (0.028 g, 0.67 mmol) and 30% H₂O₂ (304 mL, 2.68 mmol), drop-wise, maintaining the internal temperature below 5° C. The resulting cloudy solution was stirred at 0° C. for 20 min. The excess H₂O₂ was quenched by adding 1.5 M aqueous Na₂SO₃ solution (1.79 mL, 2.68 mmol) and stirred at room temperature for 5 min. The organic solvent was removed in vacuo. The resulting liquid was acidified to pH 2 by adding 1 N aqueous HCl solution. The aqueous layer was extracted with EtOAc (3×25 mL) and dried (MgSO₄). The mixture was concentrated in vacuo and then purified by ISCO silica gel column chromatography to yield (S)-2-[5-(3,5-difluoro-benzyloxy)-4′-trifluoromethyl-biphenyl-3-yl]-4-methyl-pentanoic acid (0.295 g, 92%).

¹H-NMR (CDCl₃): δ 0.93 (d, 6H), 1.51 (m, 1H), 1.72 (m, 1H), 1.98 (m, 1H), 3.72 (t, 1H), 5.09 (s, 2H), 6.76 (m, 1H), 6.98 (m, 3H), 7.07 (t, 1H), 7.17 (s, 1H), 7.66 (m, 4H); Calcd for C26H23F5O3 (M+H) 479.45, Found 479.2.

### Example 3

### (R)-2-[5-(4-fluoro-2-trifluoromethyl-benzyloxy)-4′-trifluoromethyl-biphenyl-3-yl]-4-methyl-pentanoic Acid

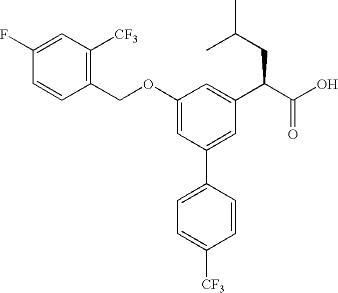

### a) 4-Benzyl-3-{2-[5-(4-fluoro-2-trifluoromethyl-benzyloxy)-4′-trifluoromethyl-biphenyl-3-yl]-4-methyl-pentanoyl}-oxazolidin-2-one

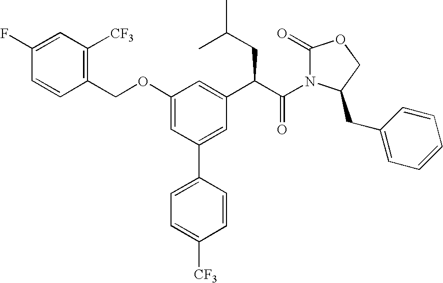

To a solution of 4-benzyl-3-[2-(5-hydroxy-4′-trifluoromethyl-biphenyl-3-yl)-4-methyl-pentanoyl]-oxazolidin-2-one (prepared in Example 1, step (k)) (0.400 g, 0.78 mmol) in acetonitrile (3.9 mL) was added 1-bromomethyl-4-fluoro-2-trifluoromethyl-benzene (0.181 mL, 1.17 mmol) and Cs₂CO₃ (0.508 g, 1.56 mmol). The resulting white suspension was stirred at room temperature for 1 h. The white solid was filtered off and the solvent was removed in vacuo to obtain relatively pure 4-benzyl-3-{2-[5-(4-fluoro-2-trifluoromethyl-benzyloxy)-4′-trifluoromethyl-biphenyl-3-yl]-4-methyl-pentanoyl}-oxazolidin-2-one.

### b) 2-[5-(4-Fluoro-2-trifluoromethyl-benzyloxy)-4′-trifluoromethyl-biphenyl-3-yl]-4-methyl-pentanoic Acid

To a solution of 4-benzyl-3-{2-[5-(4-fluoro-2-trifluoromethyl-benzyloxy)-4′-trifluoromethyl-biphenyl-3-yl]-4-methyl-pentanoyl}-oxazolidin-2-one from the previous step (0.535 g, 0.78 mmol) in THF (9 mL) was added water (3 mL). The system was cooled to 0° C. To this cold solution was added LiOH.H2O (33 mg, 0.78 mmol) and 30% H₂O₂ (0.354 mL, 3.12 mmol) and stirred at 0° C. for 20 min. The excess H₂O₂ was quenched by adding 1.5 M aqueous Na₂SO₃ solution (2.08 mL, 3.12 mmol) and stirred at room temperature for 5 min. The organic solvent was removed in vacuo. The resulting liquid was acidified to pH 2 by adding 1 N aqueous HCl solution. The aqueous layer was extracted with EtOAc (3×50 mL) and dried (MgSO₄). The mixture was concentrated in vacuo to receive a crude mixture which was purified by ISCO silica gel column chromatography to yield (R)-2-[5-(4-Fluoro-2-trifluoromethyl-benzyloxy)-4′-trifluoromethyl-biphenyl-3-yl]-4-methyl-pentanoic acid (310 mg).

¹H-NMR (CDCl₃): δ 0.92 (d, 6H), 1.52 (m, 1H), 1.71 (m, 1H), 1.99 (m, 1H), 3.73 (t, 1H), 5.27 (s, 2H), 6.98 (bs, 1H), 7.06 (bs, 1H), 7.17 (bs, 1H), 7.29 (m, 1H), 7.42 (m, 1H), 7.68 (m, 5H); Calcd for C27H23F7O3 (M+H) 529.46, Found 529.2.

### Example 4

### 2-[4′-Chloro-5-(3,5-difluoro-2-benzyloxy)-3′-trifluoromethyl-biphenyl-3-yl]-4-methyl-pentanoic Acid

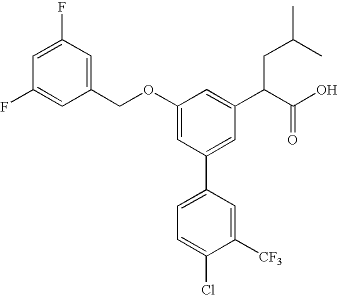

### a) 2-(3,5-Bis-benzyloxy-phenyl)-4-methyl-pent-4-enoic Acid Methyl Ester

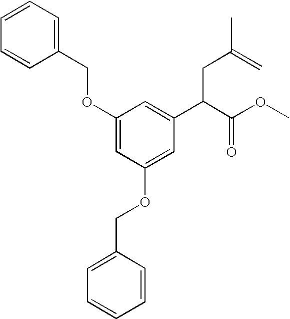

A 2M solution of LDA in THF-heptane-ethylbenzene (21.5 mL, 43.0 mmol) was added dropwise over 12 min to a stirred solution of (3,5-bis-benzyloxyphenyl)acetic acid methyl ester (prepared in Example 1, step (a)) (13.0 g, 35.9 mmol) in THF (80 mL) at −78° C. under a nitrogen atmosphere. The temperature was maintained below −70° C. for an additional 50 min, then 3-bromo-2-methylpropene (4.0 mL, 39.7 mmol) was added in one portion and the reaction mixture was warmed to 0° C. After 2 h the mixture was concentrated in vacuo, diluted with sat. aq. NH₄Cl (100 mL) and extracted with EtOAc (100 mL). The organic layer was washed with brine (100 mL), dried (MgSO₄), concentrated in vacuo, and purified by flash chromatography (silica, 0-10% EtOAc in petroleum ether) to afford the title product as a yellow oil (14.1 g, 94%).

¹H-NMR (400 MHz, CDCl₃): δ 7.42-7.25 (m, 10H), 6.58 (s, 2H), 6.52 (s, 1H), 5.02 (s, 4H), 4.74 (s, 1H), 4.66 (s, 1H), 3.74 (t, 1H), 3.64 (s, 3H), 2.79 (dd, 1H), 2.38 (dd, 1H), 1.70 (s, 3H).

### b) 2-(3-Benzyloxy-5-hydroxy-phenyl)-4-methyl-pentanoic Acid Ethyl Ester

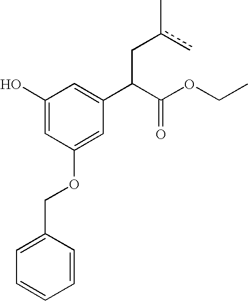

A mixture of intermediate 4a (20 g, 48 mmol), NaOH (2.3 g, 57 mmol) in EtOH (500 mL) was added 0.5 g Pd—C 10% on activated carbon under N₂, the mixture was subjected to hydrogenation under 40 psi for 30 min, at which point LC/MS indicated that the starting material was consumed. The catalyst was filtered out and EtOH was evaporated. Column chromatography (0-40% EtOAc/Heptane) gave 11.8 g (75% yield) colorless oil, as a mixture of methyl and ethyl esters and the unreduced double bond ester. MH⁺ 341 (Ethyl ester with unreduced double bond); 343 (ethyl ester with reduced isopropyl branch); 327 (methyl ester with unreduced double bond).

### c) 2-[3-Benzyloxy-5-(3,5-difluoro-benzyloxy)-phenyl]-4-methyl-pentanoic Acid Ethyl Ester

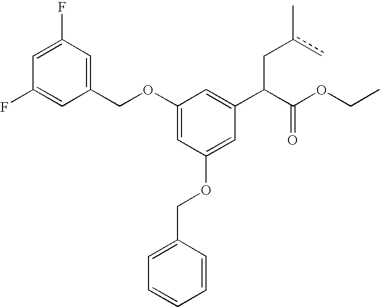

The mixture from procedure 4b (5 g, 15 mmol), K₂CO₃ (4.1 g, 30 mmol), and 3,5 di-fluoro benzyl bromide (2.9 mL, 22 mmol) in DMF (70 mL) was heated to 80° C. for one hour. DMF was removed by vacuum and the crude product was purified by column chromatography (0-30% EtOAc/heptane) to give 4.5 g product (66% yield). MH⁺ 453.1 and other molecular ions (methyl ester and the corresponding olefins).

### d) 2-[3-(3,5-Difluoro-benzyloxy)-5-trifluoromethanesulfonyloxy-phenyl]-4-methyl-pentanoic Acid Ethyl Ester

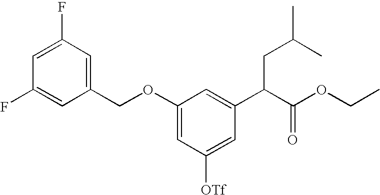

To a solution of intermediate 4c (4.5 g, 10 mmol) in MeOH (100 mL) was added 0.45 g Pd—C 10% on activated carbon under N₂; the mixture was subjected to hydrogenation under 20 psi for two hours. The catalyst was filtered out and MeOH was evaporated. Column chromatography (0-50% EtOAc/Heptane) gave 3.0 g phenol as colorless oil. The obtained phenol was dissolved in 50 mL of DCM and cooled to 0° C., followed by the addition of pyridine (2 mL, 40 mmol) and trifluoromethanesulfonic acid anhydride (2 mL, 12 mmol). The solution was stirred at 0° C. for one hour before being poured it into 1N HCl solution (20 mL), extracted with DCM (200 mL), and washed with NaHCO₃/NaCl aq. The DCM layer was dried over Mg₂SO₄ and evaporated to give 4.0 g yellow oil. (78% two steps). MH⁺ 511.2

### e) [4′-Chloro-5-(3,5-difluoro-benzyloxy)-3′-trifluoromethyl-biphenyl-3-yl]-4-methyl-pentanoic Acid Ethyl Ester

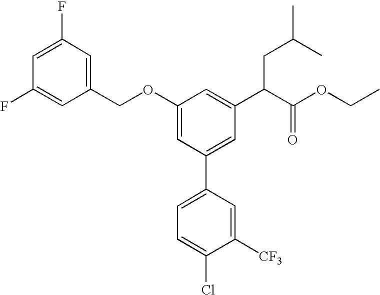

A solution of 3-trifluoro-4-chloro-benzenboronic acid (3.6 g, 16 mmol), triflate 4d (4 g, 7.8 mmol), (PPh₃)₄Pd (0.5 g, 0.4 mmol), K₂CO₃ (2.2 g, 16 mmol), in toluene/EtOH/H₂O (20/10/5 mL) was placed in a sealed reaction tube and heated to 80° C. for one hour. EtOAc (200 mL) added and washed with brine. The EtOAc layer was dried over Mg₂SO₄ and evaporated. Column chromatography (0-20%/EtOAc/Hexane) yielded 3.05 g colorless oil (74%). MH⁺ 541.3

### f) [4′-Chloro-5-(3,5-difluoro-2-benzyloxy)-3′-trifluoromethyl-biphenyl-3-yl]-4-methyl-pentanoic Acid

A solution of intermediate 4e (3 g, 5.5 mmol), 1N NaOH (16 mL) in THF/MeOH (50/50 mL) was stirred at room temperature for one day. The solution was concentrated and EtOAc (500 mL) was added. After washing with 1N HCl and brine, the EtOAc layer was dried over Mg₂SO₄ and evaporated. Column chromatography (0-30%/EtOAc/Hexane) yielded 2.7 g white solid (71%). The solid was then dissolved in EtOAc (100 mL) and added to 1N NaOH (5.26 mL, 5 mmol) and stirred at room temperature for 10 min. The solvent was then removed by vacuum and compound was obtained as its sodium salt. MH⁺ 513.2 (weak peak). ¹H NMR (300 MHz, CD₃OD): δ0.94 (d, 6H, J=6.51 Hz), δ1.5-1.67 (m, 2H), δ1.9-2.0 (m, 1H), δ3.67 (t, 1H, J=7.85 Hz), δ5.2 (s, 2H), δ6.89 (m, 1H), δ7.1 (m, 4H), δ7.27 (s, 1H), δ7.68 (d, 1H, J=8.42 Hz), δ7.85 (m, 1H), δ7.97 (d, 1H, J=2.0 Hz).

### Example 5

### 2-(3,5-Difluoro-4″-trifluoromethyl-[1,1′;3′,1″]terphenyl-5′-yl)-4-methyl-pentanoic Acid

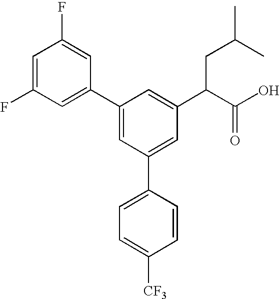

### a) 4-Methyl-2-(5-trifluoromethanesulfonyloxy-4′-trifluoromethyl-biphenyl-3-yl)-pentanoic Acid Ethyl Ester

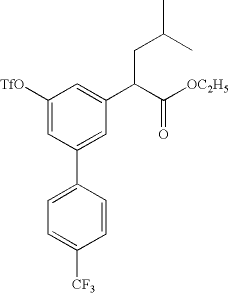

To a solution of compound 1f, 2-(5-Hydroxy-4′-trifluoromethyl-biphenyl-3-yl)-4-methyl-pentanoic acid ethyl ester, 2.8 g, 7.36 mmol) and N-phenyl-bis-(trifluoromethanesulfonimide) (3.16 g, 8.83 mmol) in THF (30 mL) under N₂ was added Et₃N (2.05 mL, 14.7 mmol). The reaction mixture was heated to reflux overnight. After cooling to room temperature, the solution was concentrated and purified by column chromatography to give the title compound (3.7 g, 98%) as a colorless thick oil; ¹H NMR (400 MHz, CHLOROFORM-D) δ ppm 0.94 (dd, J=6.60, 1.47 Hz, 6H), 1.22-1.28 (m, 3H), 1.46-1.52 (m, 1H), 1.69 (ddd, J=13.82, 7.09, 6.97 Hz, 1H), 1.98-2.06 (m, 1H), 3.75 (t, J=7.83 Hz, 1H), 4.10-4.21 (m, 2H), 7.31 (s, 1H), 7.38 (s, 1H), 7.57 (s, 1H), 7.65-7.75 (m, 4H); Calcd for C22H22F6O5S (M+H) 513.11, Found 513.

### b) 2-(3,5-Difluoro-4″-trifluoromethyl-[1,1′;3′,1″]terphenyl-5′-yl)-4-methyl-pentanoic acid

A mixture of compound 5a (50 mg, 0.098 mmol), 3,5-difluorobenzeneboronic acid (23 mg, 0.146 mmol), Pd(PPh₃)₄ (23 mg, 0.0196 mmol) and Na₂CO₃ (2N in H₂O, 0.098 mL, 0.196 mmol) in DME (1 mL) was heated at 85° C. for 3 h. After cooling to room temperature, the solution was partitioned between EtOAc and H₂O. The organic layer was dried (Na₂SO₄), concentrated, and purified by column chromatography to give an ethyl ester intermediate.

A mixture of the above intermediate and NaOH solution (2N in H₂O, 0.147 mL, 0.294 mmol) in THF-MeOH (0.6 mL-0.6 mL) was stirred for 18 h and concentrated. CH₂Cl₂ and water were added, and the mixture was acidified with 1N HCl. The organic phase was separated and the aqueous phase was extracted with CH₂Cl₂. The combined organic layers were dried, concentrated, and purified by column chromatography to give 30 mg (69%, 2 steps) of the title compound as a white solid; 1H NMR (400 MHz, MeOD) δ ppm 0.88 (dd, J=6.60, 3.18 Hz, 6H), 1.43-1.50 (m, 1H), 1.66 (ddd, J=13.82, 7.09, 6.97 Hz, 1H), 1.92-1.98 (m, 1H), 3.76 (t, J=7.83 Hz, 1H), 6.87 (tt, J=9.08, 2.29 Hz, 1H), 7.21-7.26 (m, 2H), 7.55 (d, J=1.47 Hz, 1H), 7.58-7.60 (m, 1H), 7.66-7.72 (m, 3H), 7.79 (d, J=8.07 Hz, 2H).

### Experiment 6

### 2-(3-Fluoro-4-trifluoromethoxy-4″-trifluoromethyl-[1,1′;3′,1″]terphenyl-5′-yl)-4-methyl-pentanoic Acid

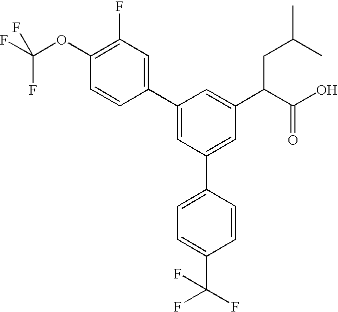

### a) 2-(3-Fluoro-4-trifluoromethoxy-4″-trifluoromethyl-[1,1′;3′,1″]terphenyl-5′-yl)-4-ethyl-pentanoic Acid Methyl Ester

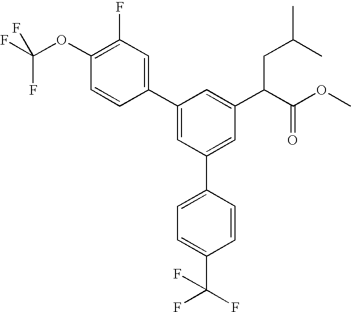

The title compound was prepared from coupling 4-methyl-2-(5-trifluoromethanesulfonyloxy-4′-trifluoromethyl-biphenyl-3-yl)-pentanoic acid methyl ester, compound 5a, with 3-fluoro-4-trifluoromethoxyphenylboronic acid under the conditions described in step as described in preparation of compound 5b in 53% yield.

¹H-NMR (400 MHz, CD₃Cl): δ ¹H-NMR (400 MHz, CDCl₃): δ 7.73 (br, s, 4H), 7.63 (t, 1H), 7.57 (t, 1H), 7.51 (t, 1H), 7.45 (m, 1H), 7.40 (m, 2H), 3.80 (m, 1H), 3.70 (s, 3H), 2.07 (m, 1H), 1.75 (m, 1H), 1.55 (m, 1H), 0.95 (d, 6H).

### b) 2-(3-Fluoro-4-trifluoromethoxy-4″-trifluoromethyl-[1,1′;3′,1″]terphenyl-5′-yl)-4-ethyl-pentanoic Acid

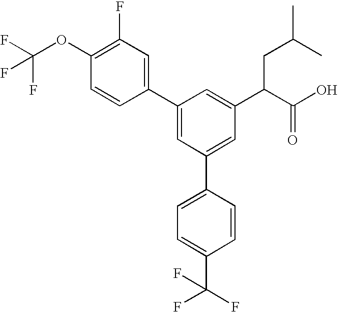

A mixture of 2-(3-fluoro-4-trifluoromethoxy-4″-trifluoromethyl-[1,1′;3′,1″]terphenyl-5′-yl)-4-ethyl-pentanoic acid methyl ester (20 mg) from the previous step in THF (1 mL), 10% LiOH in MeOH (0.3 mL) and H₂O (0.3 mL) was stirred at 30° C. for 3 h. The solution was concentrated in vacuo, diluted with H₂O, and acidified with concentrated HCl. The aqueous solution was extracted with DCM and filtered through polytetrafluoroethylene filter. The solution was concentrated in vacuo to give a solid residue. The solid was purified using reverse phase preparative HPLC (MeCN, H₂O) to give the title product (11 mg, 44%).

¹H-NMR (CD₃Cl; 400 MHz): δ 7.70 (br. s, 4H), 7.61 (t, 1H), 7.55 (t, 1H), 7.51 (t, 1H), 7.43 (m, 1H), 7.38 (m, 2H), 3.79 (m, 1H), 2.05 (m, 1H), 1.76 (m, 1H), 1.55 (m, 1H), 0.93 (d, 6H).

### Example 7

### (R)-2-(4,4″-Bis-trifluoromethyl-[1,1′;3′,1″]terphenyl-5′-yl)-4-methyl-pentanoic Acid

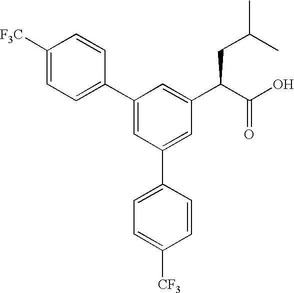

### a) Trifluoro-methanesulfonic acid 5-[1-(4-benzyl-2-oxo-oxazolidine-3-carbonyl)-3-methyl-butyl]-4′-trifluoromethyl-biphenyl-3-yl Ester

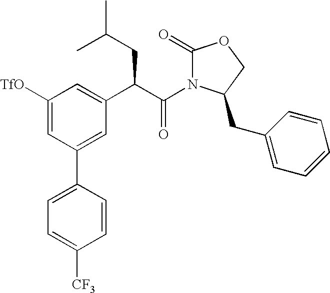

To a solution of 4-benzyl-3-[2-(5-hydroxy-4′-trifluoromethyl-biphenyl-3-yl)-4-methyl-pentanoyl]-oxazolidin-2-one (32 g, 62.6 mmol) (intermediate from Example 1k) in dichloromethane (170 mL) was added pyridine (15.0 mL). The system was cooled to 0° C. To this cold solution was added trifluoromethanesulfonic anhydride (16 mL, 94 mmol) maintaining the internal temperature below 5° C. and stirred for an additional 0.5 h at 0° C. This reaction mixture was poured to a mixture of 1 N HCl (100 mL), and wet-ice (25 g) and stirred for 0.5 h. The aqueous layer was extracted with dichloromethane (2×100 mL). Combined fractions were washed with water (2×100 mL), saturated aqueous NaHCO₃ solution (2×100 mL), and brine (2×100 mL). The organics were dried (MgSO₄) and concentrated in vacuo to receive a reddish liquid which was purified by ISCO column chromatography to yield trifluoro-methanesulfonic acid 5-[1-(4-benzyl-2-oxo-oxazolidine-3-carbonyl)-3-methyl-butyl]-4′-trifluoromethyl-biphenyl-3-yl ester (34 g, 84%).

¹H-NMR (CDCl₃): δ 0.96 (d, 3H), 0.98 (d, 3H), 1.52 (m, 1H), 1.77 (m, 1H), 2.13 (m, 1H), 2.79 (dd, 1H), 3.37 (dd, 1H), 4.14 (m, 2H), 4.67 (m, 1H), 5.33 (t, 1H), 7.20-7.38 (m, 7H), 7.70 (m, 5H); Calcd for C30H27F6NO6S (M+H) 644.15, Found 644.2.

### b) 4-Benzyl-3-[2-(4,4″-bis-trifluoromethyl-[1,1′;3′,1″]terphenyl-5′-yl)-4-methyl-pentanoyl]-oxazolidin-2-one

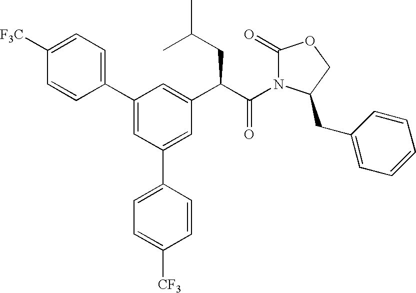

A mixture of trifluoro-methanesulfonic acid 5-[1-(4-benzyl-2-oxo-oxazolidine-3-carbonyl)-3-methyl-butyl]-4′-trifluoromethyl-biphenyl-3-yl ester from the previous step (4.03 g, 6.27 mmol), 4-(trifluoromethyl)phenylboronic acid (1.34 g, 7.05 mmol), 1,2-dimethoxyethane (24 mL) and aqueous Na₂CO₃ (2 M, 3.2 mL, 6.4 mmol) was stirred while purging with N₂ at room temperature for 10 min. To this system was added Pd[P(C₆H₅)₃]₄ (1.45 g, 1.25 mmol) and heated to reflux (95° C.) for 1 h. The red-brown mixture was diluted with EtOAc (50 mL) and washed with saturated aqueous NaHCO₃ solution (3×50 mL) and brine (2×50 mL). The organic fraction was dried (Na₂SO₄) and concentrated in vacuo. The crude mixture was purified by ISCO column chromatography to obtain 4-benzyl-3-[2-(4,4″-bis-trifluoromethyl-[1,1′;3′,1″]terphenyl-5′-yl)-4-methyl-pentanoyl]-oxazolidin-2-one (3.2 g, 79%).

¹H-NMR (CDCl₃): δ 0.97 (d, 3H), 0.99 (d, 3H), 1.58 (m, 1H), 1.80 (m, 1H), 2.17 (m, 1H), 2.79 (dd, 1H), 3.39 (dd, 1H), 4.12 (m, 2H), 4.65 (m, 1H), 5.35 (t, 1H), 7.22-7.37 (m, 5H), 7.68-7.76 (m, 11H); Calcd for C36H31F6NO3 (M+H) 640.22, Found 640.3.

### c) (R)-2-(4,4″-Bis-trifluoromethyl-[1,1′;3′,1″]terphenyl-5′-yl)-4-methyl-pentanoic Acid

To a solution of 4-benzyl-3-[2-(4,4″-bis-trifluoromethyl-[1,1′;3′,1″]terphenyl-5′-yl)-4-methyl-pentanoyl]-oxazolidin-2-one from the previous step (3.66 g, 5.7 mmol) in THF (24 mL) was added water (8 mL). The system was cooled to 0° C. To this cold solution was added LiOH.H₂O (240 mg, 5.7 mmol) and 30% H₂O₂ (1.95 mL, 17.2 mmol) and stirred at 0° C. for 15 min. The excess H₂O₂ was quenched by adding 1.5 M aqueous Na₂SO₃ solution (11.5 mL, 17.2 mmol) and stirred at room temperature for 10 min. The organic solvent was removed in vacuo. The resulting liquid was acidified to pH 2 by adding 1 N aqueous HCl solution. The aqueous layer was extracted with EtOAc (3×50 mL) and dried (MgSO₄). The mixture was concentrated in vacuo, and purified by ISCO silica gel column chromatography to yield (R)-2-(4,4″-bis-trifluoromethyl-[1,1′;3′,1″]terphenyl-5′-yl)-4-methyl-pentanoic acid (2.5 g, 92%).

¹H-NMR (CDCl₃): δ 0.96 (d, 6H), 1.59 (m, 1H), 1.79 (m, 1H), 2.08 (m, 1H), 3.83 (t, 1H), 7.58 (d, 2H), 7.69 (t, 1H), 7.72 (s, 8H); Calcd for C26H22F6O2 (M+H) 481.15, Found 481.2.

### Example 8

### (S)-2-(4,4″-Bis-trifluoromethyl-[1,1′;3′,1″]terphenyl-5′-yl)-4-methyl-pentanoic Acid

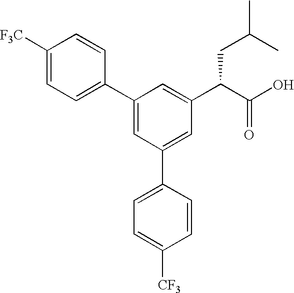

### a) Trifluoro-methanesulfonic acid 5-[1-(4-benzyl-2-oxo-oxazolidine-3-carbonyl)-3-methyl-butyl]-4′-trifluoromethyl-biphenyl-3-yl Ester

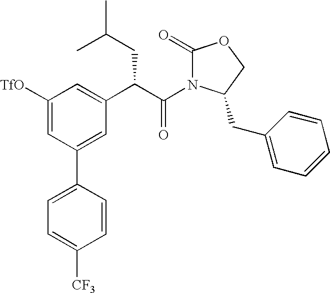

The title compound was prepared from 4-benzyl-3-[2-(5-hydroxy-4′-trifluoromethyl-biphenyl-3-yl)-4-methyl-pentanoyl]-oxazolidin-2-one (intermediate 2a) following the same procedure as for the synthesis of compound 7a.

¹H-NMR (CDCl₃): δ 0.96 (d, 3H), 0.98 (d, 3H), 1.52 (m, 1H), 1.77 (m, 1H), 2.13 (m, 1H), 2.79 (dd, 1H), 3.37 (dd, 1H), 4.14 (m, 2H), 4.67 (m, 1H), 5.33 (t, 1H), 7.20-7.38 (m, 7H), 7.70 (m, 5H); Calcd for C30H27F6NO6S (M+H) 644.15, Found 644.2.

### b) 4-Benzyl-3-[2-(4,4″-bis-trifluoromethyl-[1,1′;3′,1″]terphenyl-5′-yl)-4-methyl-pentanoyl]-oxazolidin-2-one

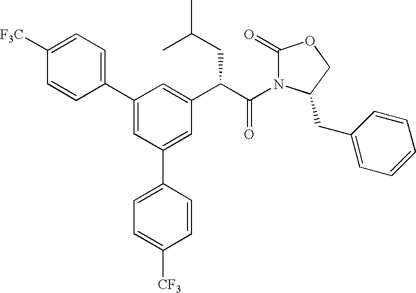

The title compound was prepared from trifluoro-methanesulfonic acid 5-[1-(4-benzyl-2-oxo-oxazolidine-3-carbonyl)-3-methyl-butyl]-4′-trifluoromethyl-biphenyl-3-yl ester from the previous step following the same procedure as for the synthesis of compound 7b.

¹H-NMR (CDCl₃): δ 0.97 (d, 3H), 0.99 (d, 3H), 1.58 (m, 1H), 1.80 (m, 1H), 2.17 (m, 1H), 2.79 (dd, 1H), 3.39 (dd, 1H), 4.12 (m, 2H), 4.65 (m, 1H), 5.35 (t, 1H), 7.22-7.37 (m, 5H), 7.68-7.76 (m, 11H); Calcd for C36H31F6NO3 (M+H) 640.22, Found 640.3.

### c) (S)-2-(4,4″-Bis-trifluoromethyl-[1,1′;3′,1″]terphenyl-5′-yl)-4-methyl-pentanoic Acid

The title compound was prepared from 4-benzyl-3-[2-(4,4″-bis-trifluoromethyl-[1,1′;3′, 1″]terphenyl-5′-yl)-4-methyl-pentanoyl]-oxazolidin-2-one from the previous step following the same procedure as for the synthesis of compound 7.

¹H-NMR (CDCl₃): δ 0.96 (d, 6H), 1.59 (m, 1H), 1.79 (m, 1H), 2.08 (m, 1H), 3.83 (t, 1H), 7.58 (d, 2H), 7.69 (t, 1H), 7.72 (s, 8H); Calcd for C26H22F6O2 (M+H) 481.15, Found 481.2.

### Example 9

### 2-[5-(3,5-Difluoro-phenylamino)-4′-trifluoromethyl-biphenyl-3-yl]-4-methyl-pentanoic Acid

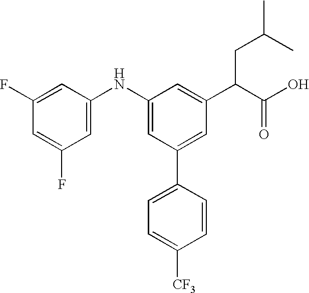

A mixture of compound 5a (50 mg, 0.098 mmol), 3,5-difluoro-aniline (20 mg, 0.156 mmol), Pd(OAc)₂ (6.6 mg, 0.029 mmol), racemic-2-(di-t-butylphosphino)-1,1′-binaphthyl (35 mg, 0.088 mmol) and sodium tert-butoxide (NaOt-Bu) (11.3 mg, 0.12 mmol) in toluene (1.5 mL) was heated to 85° C. for 17 h. After cooling to room temperature, the solution was partitioned between EtOAc and H₂O. The organic layer was dried (Na₂SO₄), concentrated and purified by column chromatography to give an ethyl ester intermediate. A mixture of the above obtained intermediate and NaOH (2N in H₂O, 0.147 mL, 0.294 mmol) in THF-MeOH (0.6 mL-0.6 mL) was stirred for 18 h and concentrated. CH₂Cl₂ and water were added, and the mixture was acidified with 1N HCl. The organic phase was separated and the aqueous phase was extracted with CH₂Cl₂. The combined organic layers were dried, concentrated, and purified by column chromatography to give 38 mg (84%, 2 steps) of the title compound as a white solid; 1H NMR (400 MHz, CHLOROFORM-D) δ ppm 0.91-1.00 (m, 6H), 1.51-1.62 (m, 1H), 1.70-1.80 (m, 1H), 1.99 (dd, J=7.83, 5.87 Hz, 1H), 3.71 (t, J=7.70 Hz, 1H), 6.01 (brs, 1H), 6.30-6.40 (m, 1H), 6.50-6.60 (m, 2H), 7.13 (d, J=1.71 Hz, 1H), 7.18-7.29 (m, 2H), 7.62-7.72 (m, 4H); Calcd for C25H22F5NO2 (M+H) 464.16, Found 464.

### Example 10

### 4-Methyl-2-[4′-trifluoromethyl-5-(4-trifluoromethyl-phenylamino)-biphenyl-3-yl]-pentanoic Acid

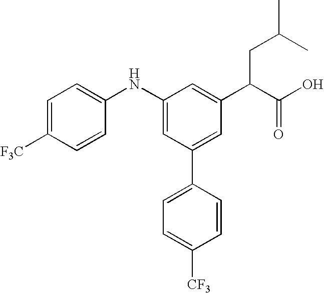

The title compound was prepared from 4-trifluoromethyl-aniline and compound 5a under the condition described in Example 9; ¹H NMR (400 MHz, CHLOROFORM-D) δ ppm 0.95 (d, J=6.36 Hz, 6H), 1.57 (dt, J=13.27, 6.69 Hz, 1H), 1.74 (ddd, J=13.69, 7.21, 6.97 Hz, 1H), 1.96-2.05 (m, 1H), 3.66-3.76 (m, 1H), 7.07-7.12 (m, 2H), 7.14-7.20 (m, 2H), 7.25-7.29 (m, 1H) 7.50 (d, J=8.56 Hz, 2H) 7.62-7.72 (m, 4H); Calcd for C26H23F6NO2 (M+H) 496.16, Found 496.

### Example 11

### 2-[5-(4-Isopropyl-phenylamino)-4′-trifluoromethyl-biphenyl-3-yl]-4-methyl-pentanoic Acid

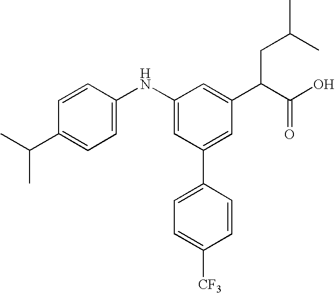

The title compound was prepared from 4-isopropyl-aniline and compound 5a under the condition described in Example 9; H NMR (400 MHz, CHLOROFORM-D) δ ppm 0.87-0.98 (m, 6H), 1.20-1.31 (m, 6H), 1.52-1.63 (m, 1H), 1.72 (ddd, J=13.69, 7.21, 6.97 Hz, 1H), 1.94-2.05 (m, 1H), 2.88 (dt, J=13.69, 6.85 Hz, 1H), 3.67 (t, J=7.70 Hz, 1H), 6.99-7.10 (m, 4H), 7.11-7.20 (m, 3H), 7.59-7.69 (m, 4H); Calcd for C28H30F3NO2 (M+H) 470.22, Found 470.

### Example 12

### (R)2-[5-(2,5-Bis-trifluoromethyl-phenylamino)-4′-trifluoromethyl-biphenyl-3-yl]-4-methyl-pentanoic Acid

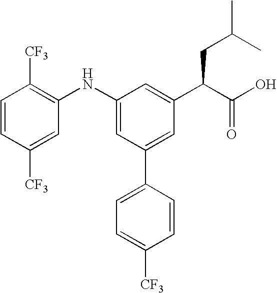

### a) 4-Benzyl-3-{2-[5-(2,5-bis-trifluoromethyl-phenylamino)-4′-trifluoromethyl-biphenyl-3-yl]-4-methyl-pentanoyl}-oxazolidin-2-one

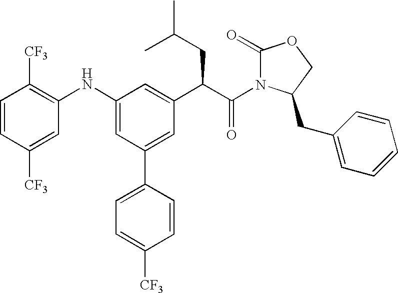

To a solution of trifluoro-methanesulfonic acid 5-[1-(4-benzyl-2-oxo-oxazolidine-3-carbonyl)-3-methyl-butyl]-4′-trifluoromethyl-biphenyl-3-yl ester (compound 7a, 4.84 g, 7.53 mmol) in toluene (38 mL) in a sealed tube was added 2,5-bis-trifluoromethyl-phenylamine (1.42 mL, 9.04 mmol), [1,1′]binaphthalenyl-2-yl-di-tert-butyl-phosphane (300 mg, 0.75 mmol), Pd(OAc)₂ (169 mg, 0.75 mmol) and KOtBu (7.53 mL of 1.0 M solution in THF, 7.53 mmol). The reaction mixture was heated to 120° C. for 1 h. To this was added another portion each of [1,1′]binaphthalenyl-2-yl-di-tert-butyl-phosphane (300 mg, 0.75 mmol), Pd(OAc)₂ (169 mg, 0.75 mmol) and KOtBu (3.77 mL of 1.0 M solution in THF, 3.77 mmol) and heated for further 1 h. The system was cooled to room temperature and quenched by slow addition of water. The mixture was extracted with EtOAc (3×50 mL). The organic phase was washed with saturated NaHCO₃ solution and brine. The organic fraction was dried (Na₂SO₄) and concentrated in vacuo. The crude mixture was purified by ISCO column chromatography to obtain. 4-benzyl-3-{2-[5-(2,5-bis-trifluoromethyl-phenylamino)-4′-trifluoromethyl-biphenyl-3-yl]-4-methyl-pentanoyl}-oxazolidin-2-one (2.32 g, 43%).

¹H-NMR (CDCl₃): δ 0.97 (d, 3H), 0.99 (d, 3H), 1.56 (m, 1H), 1.76 (m, 1H), 2.10 (m, 1H), 2.78 (dd, 1H), 3.37 (dd, 1H), 4.14 (m, 2H), 4.65 (m, 1H), 5.28 (t, 1H), 6.32 (s, 1H), 7.17-7.40 (m, 9H), 7.59 (s, 1H), 7.69 (m, 5H); Calcd for C37H31F9N2O3 (M+H) 723.22, Found 723.3.

### b) (R)-2-[5-(2,5-Bis-trifluoromethyl-phenylamino)-4′-trifluoromethyl-biphenyl-3-yl]-4-methyl-pentanoic Acid

To a solution of 4-benzyl-3-{2-[5-(2,5-bis-trifluoromethyl-phenylamino)-4′-trifluoromethyl-biphenyl-3-yl]-4-methyl-pentanoyl}-oxazolidin-2-one (2.55 g, 3.53 mmol), from the previous step, in THF (15 mL) was added water (5 mL). The reaction mixture was cooled to 0° C. To this cold solution was added LiOH.H2O (148 mg, 3.53 mmol) and 30% H₂O₂ (1.20 mL, 10.59 mmol) and stirred at 0° C. for 15 min. The excess H₂O₂ was quenched by adding 1.5 M aqueous Na₂SO₃ solution (7.06 mL, 10.59 mmol) and stirred at room temperature for 10 min. The organic solvent was removed in vacuo. The resulting liquid was acidified to pH 2 by adding 1 N aqueous HCl solution. The aqueous layer was extracted with EtOAc (3×50 mL) and dried (MgSO₄). The mixture was concentrated in vacuo, and purified by ISCO silica gel column chromatography to yield (R)2-[5-(2,5-Bis-trifluoromethyl-phenylamino)-4′-trifluoromethyl-biphenyl-3-yl]-4-methyl-pentanoic acid (1.15 g, 58%).

¹H-NMR (CDCl₃): δ 0.94 (d, 6H), 1.56 (m, 1H), 1.76 (m, 1H), 2.00 (m, 1H), 3.74 (t, 1H), 6.32 (s, 1H), 7.17-7.29 (m, 4H), 7.60 (s, 1H), 7.67 (m, 5H); Calcd for C27H22F9NO2 (M+H) 564.15, Found 564.3.

### Example 13

### 4-Methyl-2-{5-[methyl-(4-trifluoromethyl-phenyl)-amino]-4′-trifluoromethyl-biphenyl-3-yl}-pentanoic Acid

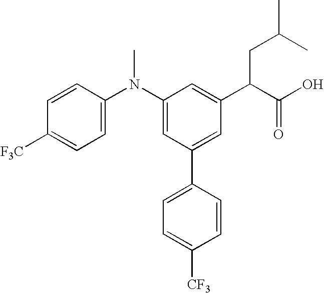

### a) 4-Methyl-2-[4′-trifluoromethyl-5-(4-trifluoromethyl-phenylamino)-biphenyl-3-yl]-pentanoic Acid Ethyl Ester

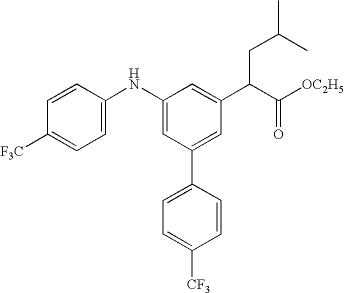

A mixture of compound 5a (50 mg, 0.098 mmol), 4-trifluoromethyl-aniline (25 mg, 0.156 mmol), Pd(OAc)₂ (6.6 mg, 0.029 mmol), racemic-2-(di-t-butylphosphino)-1,1′-binaphthyl (35 mg, 0.088 mmol) and NaOt-Bu (11.3 mg, 0.12 mmol) in toluene (1.5 mL) was heated at 85° C. for 17 h. After cooling to room temperature, the solution was partitioned between EtOAc and H₂O. The organic layer was dried (Na₂SO₄), concentrated and purified by column chromatography to give the title compound.

### b) 4-Methyl-2-{5-[methyl-(4-trifluoromethyl-phenyl)-amino]-4′-trifluoromethyl-biphenyl-3-yl}-pentanoic Acid

To a solution of the above aniline ester intermediate (40 mg, 0.076 mmol) in acetonitrile (1 mL) was added MeI (0.048 mL, 0.76 mmol) and Et₃N (0.032 mL, 0.228 mmol). The mixture was heated to 85° C. for 17 h. After cooling to room temperature, the solution was partitioned between EtOAc and H₂O. The organic layer was dried (Na₂SO₄), concentrated and purified by column chromatography to give an ethyl ester intermediate.

A mixture of the above intermediate and NaOH solution (2N in H₂O, 0.114 mL, 0.228 mmol) in THF-MeOH (0.6 mL-0.6 mL) was stirred for 18 h and concentrated. CH₂Cl₂ and water were added, and the mixture was acidified with 1N HCl. The organic phase was separated and the aqueous phase was extracted with CH₂Cl₂. The combined organic layers were dried, concentrated, and purified by column chromatography to give 30 mg (60%, 3 steps) of the title compound as a white solid; 1H NMR (400 MHz, MeOD) δ 0.86 (d, J=6.60 Hz, 6H), 1.41-1.50 (m, 1H), 1.57-1.65 (m, 1H), 1.84-1.92 (m, 1H), 3.22 (s, 3H), 3.62-3.70 (m, 1H), 6.88 (d, J=8.80 Hz, 2H), 7.17 (d, J=1.71 Hz, 1H), 7.30-7.39 (m, 4H), 7.62-7.72 (m, 4H); Calcd for C27H25F6NO2 (M+H) 510.18, Found 510.

### Example 14

### 4-Methyl-2-{5-[(3-methyl-butyl)-(4-trifluoromethyl-phenyl)-amino]-4′-trifluoromethyl-biphenyl-3-yl}-pentanoic Acid

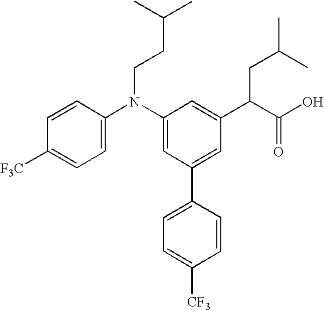

A mixture of compound 13a, 1-iodo-3-methyl-butane, and Cs₂CO₃ were reacted according to the procedure of Example 13, followed by ester hydrolysis to give the title compound; 1H NMR (400 MHz, MeOD) δ 0.87-0.96 (m, 12H), 1.51-1.74 (m, 5H), 1.96 (dt, J=13.69, 7.58 Hz, 1H), 3.75 (t, J=7.83 Hz, 1H), 3.80-3.86 (m, 2H), 6.92 (d, J=8.56 Hz, 2H), 7.22-7.26 (m, 1H), 7.36-7.46 (m, 4H), 7.70-7.78 (m, 4H); Calcd for C31H33F6NO2 (M+H) 566.24, Found 566.

### Example 15

### 2-{5-[(4-Chloro-phenyl)-(3-methyl-butyl)-amino]-4′-trifluoromethyl-biphenyl-3-yl}-4-methyl-pentanoic Acid

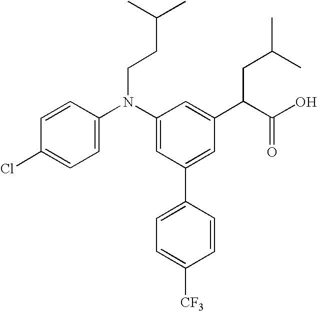

Using the reaction conditions from Example 13a, compound 5a was reacted with 4-chloro-aniline; subsequent reactions as described in Example 14 gave the title compound; 1H NMR (400 MHz, MeOD) δ ppm 0.78-0.89 (m, 12H), 1.41-1.53 (m, 3H), 1.56-1.64 (m, 2H), 1.83 (dt, J=13.51, 7.55 Hz, 1H), 3.59 (t, J=7.83 Hz, 1H), 3.68-3.74 (m, 2H), 6.91-6.96 (m, 3H), 7.04 (s, 1H), 7.11 (s, 1H), 7.15-7.19 (m, 2H), 7.63 (s, 4H); Calcd for C30H33ClF3NO2 (M+H) 532.22, Found 532.

### Example 16

### 2-[5-(3-Isopropyl-phenoxy)-4′-trifluoromethyl-biphenyl-3-yl]-4-methyl-pentanoic Acid

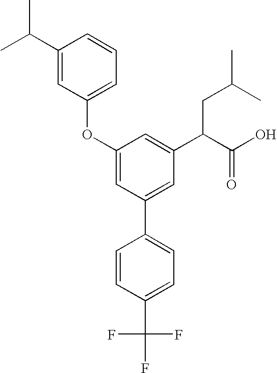

### a) 2-[5-(3-Isopropyl-phenoxy)-4′-trifluoromethyl-biphenyl-3-yl]-4-methyl-pentanoic Acid Methyl Ester

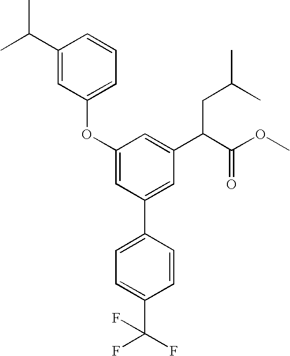

A mixture of 2-(5-hydroxy-4′-trifluoromethyl-biphenyl-3-yl)-4-methyl-pentanoic acid methyl ester, compound 1f, (50 mg, 0.14 mmol), 3-isopropylphenyl boronic acid (45 mg, 0.27 mmol), copper acetate (26 mg, 0.14 mmol), triethylamine (57 μL, 0.4 mmol) and powdered 4 Å molecular sieves in DCM (1 mL) were stirred at room temperature for 2 days. The reaction mixture was concentrated in vacuo. Purification by flash chromatography (EtOAc: petroleum ether) gave the title compound (32 mg, 48%).

### b) 2-[5-(3-Isopropyl-phenoxy)-4′-trifluoromethyl-biphenyl-3-yl]-4-methyl-pentanoic Acid

A mixture of 2-[5-(3-isopropyl-phenoxy)-4′-trifluoromethyl-biphenyl-3-yl]-4-methyl-pentanoic acid methyl ester from the previous step (33 mg, 0.07 mmol), THF (0.6 mL), 10% aq. LiOH (0.2 mL) and MeOH (0.6 mL) was stirred at 30° C. for 3 h. The solution was concentrated and the residue was diluted with H₂O (1 mL) and then acidified with concentrated HCl. The aqueous solution was extracted with DCM (3×1 mL) and the organic layers were filtered through a PTFE filter. The solution was concentrated in vacuo to give a solid residue. The solid was purified using reverse phase preparative HPLC (MeCN, H₂O) to afford the title compound (21.6 mg, 67%).

¹H-NMR (CD₃Cl; 400 MHz): δ 7.64 (dd, 4H), 7.29-7.23 (m, 2H), 7.11 (br. s, 1H), 7.05 (br. s), 7.00 (dd, 1H), 6.96-6.93 (m, 1H), 6.84 (d, 1H), 3.80-3.65 (m, 1H), 2.95-2.85 (m, 1H), 2.02-1.90 (m, 1H), 1.80-1.65 (m, 1H), 1.60-1.45 (m, 1H), 1.23 (d, 6H), 0.92 (d, 6H).

### Example 17

### 4-Methyl-2-[4′-chloro-3′-trifluoromethyl-5-(3-fluoro-5-trifluoromethylphenoxy)-biphenyl-3-yl]-pentanoic Acid

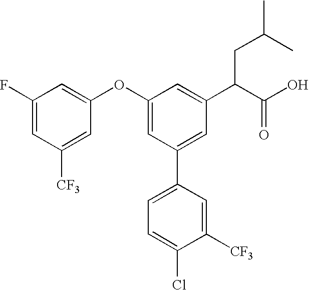

### a) 2-(4′-Chloro-5-hydroxy-3′-trifluoromethyl-biphenyl-3-yl)-4-methyl-pentanoic Acid Methyl Ester

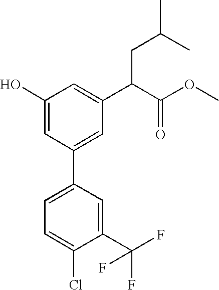

The title compound was prepared from compound 1c (under the conditions described in Example 1 steps (d-f) using 4-chloro-5-trifluoromethylphenylboronic acid in step (d).

¹H-NMR (400 MHz, CDCl₃): δ 7.84 (s, 1H), 7.65 (d, 1H), 7.55 (d, 1H), 7.04 (s, 1H), 6.92 (m, 1H), 6.86 (m, 1H), 4.98 (br s, 1H), 3.68 (m, 4H), 1.97 (m, 1H), 1.68 (m, 1H), 1.49 (m, 1H), 0.92 (d, 6H); Mass Spectrum (m/z, ESI) 399 (M−H)

### b) 4-Methyl-2-[4′-chloro-3′-trifluoromethyl-5-(3-fluoro-5-trifluoromethylphenoxy)-biphenyl-3-yl]-pentanoic Acid Methyl Ester

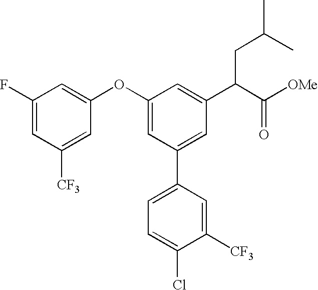

The title compound was prepared in 50% yield from 2-(5-hydroxy-4′-chloro-3′-trifluoromethyl-biphenyl-3-yl)-4-methyl-pentanoic acid methyl ester (from the previous example) and 3-fluoro-5-trifluoromethylphenylboronic acid under the conditions described in Example 16 step (a).

### c) 4-Methyl-2-[4′-chloro-3′-trifluoromethyl-5-(3-fluoro-5-trifluoromethylphenoxy)-biphenyl-3-yl]-pentanoic Acid

The title compound was prepared in 90% yield from 4-methyl-2-[4′-chloro-3′-trifluoromethyl-5-(3-fluoro-5-trifluoromethylphenoxy)-biphenyl-3-yl]-pentanoic acid methyl ester (from the previous example) under the conditions described in Example 16 step (b).

¹H-NMR (400 MHz, CDCl₃): δ 7.79 (d, 1H), 7.55 (d, 1H), 7.50 (d, 1H), 7.28 (d, 1H), 7.05 (m, 4H), 6.82 (d, 1H), 3.59 (t, 1H), 1.84 (m, 1H), 1.64 (m, 1H), 1.41 (m, 1H), 0.83 (d, 6H).

### Example 18

### 2-[5-(3-Fluoro-5-trifluoromethyl-phenoxy)-4′-trifluoromethyl-biphenyl-3-yl]-4-methyl-pentanoic Acid

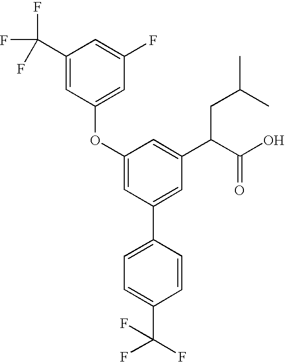

### a) 2-[5-(3-Fluoro-5-trifluoromethyl-phenoxy)-4′-trifluoromethyl-biphenyl-3-yl]-4-methyl-pentanoic Acid Methyl Ester

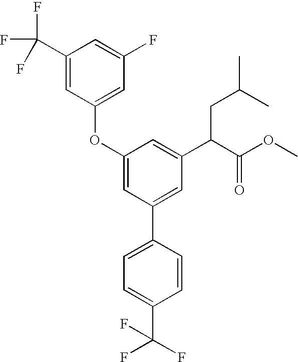

The title compound was prepared from 2-(5-hydroxy-4′-trifluoromethyl-biphenyl-3-yl)-4-methyl-pentanoic acid methyl ester (prepared in Example 1, step (f)) and 3-fluoro,5-trifluoromethylbenzene boronic acid under the conditions described in Example 16, step (a).

### b) 2-[5-(4-Chloro-phenoxy)-4′-trifluoromethyl-biphenyl-3-yl]-4-methyl-pentanoic Acid

The title compound was prepared from 2-[5-(3-fluoro-5-trifluoromethyl-phenoxy)-4′-trifluoromethyl-biphenyl-3-yl]-4-methyl-pentanoic acid methyl ester (from the previous step) under the conditions described in Example 16, step (b).

¹H-NMR (400 MHz, CDCl₃): δ 7.68 (d, 2H, J=8.3 Hz), 7.63 (d, 2H, J=8.1 Hz), 7.37 (m, 1H), 7.16 (m, 1H), 7.08 (m, 1H), 7.06 (m, 2H), 6.87 (dt, 2H, J=9.6, 2.3 Hz), 3.71 (t, 1H, J=7.8 Hz), 1.95 (m, 1H), 1.71 (m, 1H), 1.50 (m, 1H), 0.90 (dd, 6H, J=6.6, 2.3 Hz).

### Example 19

### 2-[5-(2,6-Difluoro-pyridin-4-yl)-4′-trifluoromethyl-biphenyl-3-yl]-4-methyl-pentanoic Acid

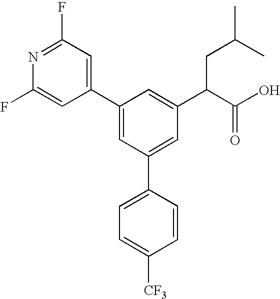

A mixture of compound 5a (40 mg, 0.078 mmol), 2,6-difluoro-pyridin-4-boronic acid (52.5 mg, 0.117 mmol), Pd(PPh₃)₄ (18 mg, 0.0156 mmol) and Na₂CO₃ solution (2N in H₂O, 0.078 mL, 0.156 mmol) in DME (1 mL) was heated to 85° C. for 3 h. After cooling to room temperature, the solution was partitioned between EtOAc and H₂O. The organic layer was dried (Na₂SO₄), concentrated and purified by column chromatography to give an ethyl ester intermediate.

To a solution of the above intermediate in THF—H₂O (1 mL-0.3 mL) was added LiOH.H₂O (32.8 mg, 0.78 mmol). The reaction mixture was stirred at room temperature for 24 h and concentrated. CH₂Cl₂ and water were added, and the mixture was acidified with 1N HCl. The organic phase was separated and the aqueous phase was extracted with CH₂Cl₂. The combined organic layers were dried, concentrated, and purified by column chromatography to give 22 mg (63%, 2 steps) of the title compound as a white solid; ¹H NMR (400 MHz, MeOD) δ 0.83-0.92 (m, 6H), 1.48 (dt, J=13.39, 6.63 Hz, 1H), 1.69 (ddd, J=13.82, 7.09, 6.97 Hz, 1H), 1.99 (ddd, J=13.57, 7.83, 7.70 Hz, 1H), 3.81 (t, J=7.83 Hz, 1H), 7.24-7.31 (m, 2H), 7.70 (ddd, J=6.05, 3.97, 1.96 Hz, 4H), 7.78-7.88 (m, 3H); Calcd for C24H20F5NO2 (M+H) 450.14, Found 450.

### Example 20

### 4-Methyl-2-[4′-trifluoromethyl-5-(5-trifluoromethyl-pyridin-2-yl)-biphenyl-3-yl]-pentanoic Acid

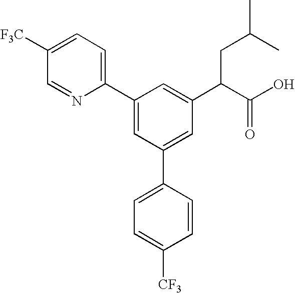

Using the procedure of Example 19, reaction of compound 5a with 5-(trifluoromethyl)pyridine-2-boronic acid pinacol ester, followed by 2N NaOH in H₂O and MeOH hydrolysis gave the title compound; ¹H NMR (400 MHz, MeOD) δ 0.88 (ddd, J=19.81, 6.48, 3.55 Hz, 6H), 1.49 (dt, J=13.39, 6.63 Hz, 1H), 1.70 (ddd, J=13.82, 7.09, 6.97 Hz, 1H), 1.93-2.04 (m, 1H), 3.81 (t, J=7.70 Hz, 1H), 7.71 (ddd, J=3.91, 2.32, 2.08 Hz, 3H), 7.83 (d, J=8.31 Hz, 2H), 8.04-8.14 (m, 3H), 8.22 (t, J=1.59 Hz, 1H), 8.89 (s, 1H); Calcd for C25H21F6NO2 (M+H) 482.15, Found 482.1.

### Example 21

### 2-{5-[1-(3,5-Difluoro-phenyl)-4-methyl-pentyloxy]-4′-trifluoromethyl-biphenyl-3-yl}-4-methyl-pentanoic Acid

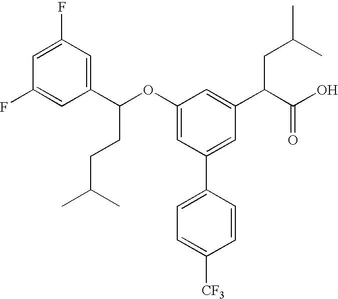

### a) 1-(3,5-Difluoro-phenyl)-4-methyl-pentan-1-ol

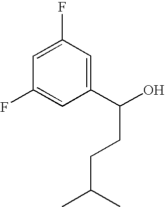

A flame-dried 3-neck flask under N₂ was charged with 1-bromo-3-methyl-butane (1.5 g, 9.9 mmol), magnesium turnings (241 mg, 9.9 mmol), a catalytic amount of HgCl₂, and dry ether (12 mL). The reaction mixture was heated to 40° C. for 3 h and cooled to generate 3-methyl-n-butylmagnesium bromide.

To a solution of 3,5-difluoro-benzaldehyde (300 mg, 2.11 mmol) in THF (2 mL) was added half of the above freshly made Grignard reagent. The reaction mixture was stirred for 17 h and partitioned between EtOAc and saturated NH₄Cl solution. The organic layer was dried (Na₂SO₄), concentrated and purified by column chromatography to give 150 mg (66%) of the title compound, as a clear oil; ¹H NMR (400 MHz, CHLOROFORM-D) δ 0.79-0.89 (m, 17H) 1.50-1.60 (m, 8H) 2.79-2.86 (m, 5H) 6.88-6.97 (m, 3H) 7.35 (s, 1H) 7.37 (dd, J=6.11, 1.71 Hz, 4H); Calcd for C12H16F2O (M+H) 215.12, Found 215.1.

### b) 2-{5-[1-(3,5-Difluoro-phenyl)-4-methyl-pentyloxy]-4′-trifluoromethyl-biphenyl-3-yl}-4-methyl-pentanoic Acid

A solution of compound 21a (50 mg, 0.131 mmol) compound 1f (50 mg, 0.233 mmol), Ph₃P (61 mg, 0.233 mmol) and diisopropylazodicarboxylate (0.047 mL, 0.233 mmol) in THF (1.5 mL) was stirred at room temperature for 24 h and concentrated. The residue was purified by column chromatography to give an ethyl ester intermediate.

The ethyl ester intermediate was hydrolyzed following the same hydrolysis procedure as in Example 13 to give the title compound; ¹H NMR (400 MHz, CHLOROFORM-D) δ 0.83-0.92 (m, 12H), 1.22-1.32 (m, 1H), 1.36-1.48 (m, 2H), 1.54-1.62 (m, 2H), 1.79-2.00 (m, 3H), 3.56-3.66 (m, 1H), 5.05-5.10 (m, 1H), 6.60-6.70 (m, 1H), 6.82 (d, J=1.47 Hz, 1H), 6.86-6.95 (m, 3H), 7.05-7.09 (m, 1H), 7.54-7.60 (m, 2H), 7.63-7.68 (m, 2H); Calcd for C31H33F5O3 (M+Na⁺) 571.23, Found 571.2.

### Example 22

### 2-[5-(3,5-Dichloro-benzoyl)-4′-trifluoromethyl-biphenyl-3-yl]-4-methyl-pentanoic Acid

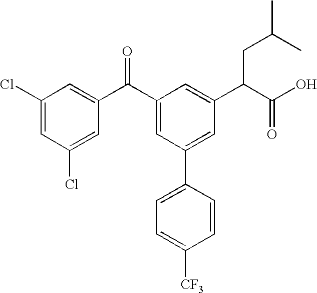

A mixture of compound 5a (100 mg, 0.195 mmol), 3,5-dichloro-phenyl-boronic acid (63 mg, 0.33 mmol), Pd(dppf)₂Cl₂ (14.3 mg, 0.020 mmol), K₂CO₃ (81 mg, 0.585 mmol) and KI (97 mg, 0.585 mmol) in anisole (2 mL) at 85° C. under a CO atmosphere using a balloon filled with CO gas was heated for 24 h. After cooling to room temperature, the solution was partitioned between EtOAc and H₂O. The organic layer was dried (Na₂SO₄), concentrated and purified by column chromatography to give an ethyl ester intermediate.

The above ester intermediate was hydrolyzed under the condition described in Example 13 to give the title compound; ¹H NMR (400 MHz, MeOD) δ 0.96 (dd, J=6.60, 1.47 Hz, 6H), 1.53 (ddd, J=13.57, 6.72, 6.60 Hz, 1H), 1.77 (ddd, J=13.82, 7.70, 6.36 Hz, 1H), 2.00 (dt, J=13.51, 7.67 Hz, 1H), 3.88 (t, J=7.83 Hz, 1H), 7.71-7.80 (m, 6H), 7.83-7.88 (m, 2H), 7.95-7.98 (m, 2H); Calcd for C26H21Cl2F3O3 (M+H) 509.08, Found 509.1.

### Example 23

### 2-[5-(2-Biphenyl-4-yl-vinyl)-4′-trifluoromethyl-biphenyl-3-yl]-4-methyl-pentanoic Acid

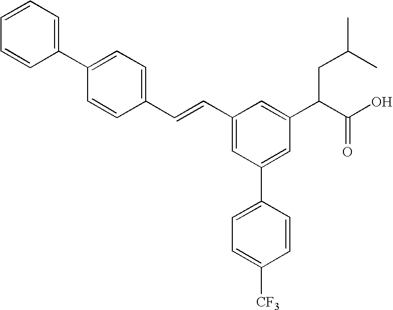

A mixture of compound 5a (60 mg, 0.117 mmol), trans-2-(4-biphenyl)-vinyl-boronic acid (45 mg, 0.199 mmol), Pd(dppf)₂Cl₂ (10 mg, 0.0117 mmol) and K₂CO₃ (32.3 mg, 0.234 mmol) in 1,4-dioxane-water (0.8 mL-0.8 mL) was heated to 85° C. for 15 h. After cooling to room temperature, the solution was partitioned between EtOAc and H₂O. The organic layer was dried (Na₂SO₄), concentrated and purified by column chromatography to give an ethyl ester intermediate.

The above ester intermediate was hydrolyzed following the same hydrolysis procedure of Example 13 to give the title compound; ¹H NMR (400 MHz, MeOD) δ 0.88-0.99 (m, 6H), 1.57 (dt, J=13.39, 6.63 Hz, 1H), 1.74 (ddd, J=13.82, 7.09, 6.97 Hz, 1H), 1.98-2.09 (m, 2H), 3.80 (t, J=7.70 Hz, 1H), 7.26-7.34 (m, 3H), 7.42 (t, J=7.58 Hz, 2H), 7.52 (s, 1H), 7.57-7.65 (m, 7H), 7.74 (d, J=9.05 Hz, 3H), 7.78-7.84 (m, 2H); Calcd for C33H29F3O2 (M+H) 515.21, Found 515.2.

### Example 24

### 2-[5-(2-Biphenyl-4-yl-ethyl)-4′-trifluoromethyl-biphenyl-3-yl]-4-methyl-pentanoic Acid

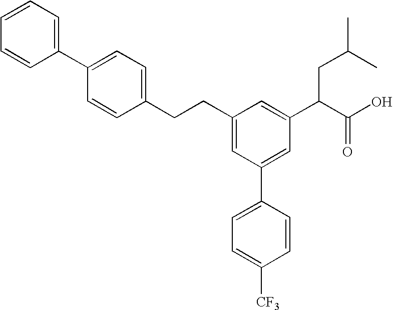

A mixture of compound 23 (20 mg, 0.039 mmole), 10% Pd/C (10 mg), and MeOH (5 mL) was hydrogenated under H₂ (40 psi) in par-shaker for 20 h. The resulting reaction mixture was filtered through a celite pad and the filtrate was concentrated to give the title compound (19 mg, 98%) as a white solid; ¹H NMR (300 MHz, MeOD) δ 0.83-0.92 (m, 6H), 1.46 (ddd, J=13.38, 6.59, 6.41 Hz, 1H), 1.63 (ddd, J=13.75, 7.16, 6.97 Hz, 1H), 1.87-1.98 (m, 1H), 2.94-3.07 (m, 4H), 3.68 (t, J=7.72 Hz, 1H), 7.15-7.23 (m, 3H), 7.25-7.33 (m, 2H), 7.36-7.44 (m, 3H), 7.50 (d, J=8.29 Hz, 2H), 7.57 (d, J=7.54 Hz, 2H), 7.64-7.71 (m, 4H); Calcd for C33H31F3O2 (M+Na) 539.23, Found 539.2.

### Example 25

### 4-Methyl-2-[4′-trifluoromethyl-5-(3-trifluoromethyl-benzoylamino)-biphenyl-3-yl]-pentanoic Acid

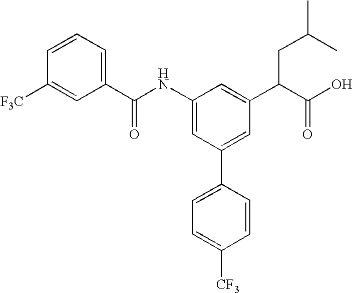

A mixture of compound 5a (40 mg, 0.078 mmol), 3-trifluoromethyl-benzamide (25 mg, 0.132 mmol), Pd(OAc)₂ (6.6 mg, 0.029 mmol), racemic-2-(di-t-butylphosphino)-1,1′-binaphthyl (35 mg, 0.088 mmol), and NaOt-Bu (11.3 mg, 0.12 mmol) in toluene (1.5 mL) was heated to 85° C. for 17 h. After cooling to room temperature, the solution was partitioned between EtOAc and H₂O. The organic layer was dried (Na₂SO₄), concentrated and purified by column chromatography to give an ester intermediate.

The above ester intermediate was hydrolyzed following the same hydrolysis procedure of Example 13 to give the title compound; ¹H NMR (400 MHz, MeOD) δ 0.97 (dd, J=6.60, 2.20 Hz, 6H), 1.56 (dt, J=13.39, 6.63 Hz, 1H), 1.75 (ddd, J=13.69, 7.21, 6.97 Hz, 1H), 2.02 (dt, J=13.69, 7.70 Hz, 1H), 3.77 (t, J=7.70 Hz, 1H), 7.45 (s, 1H), 7.71-7.79 (m, 4H), 7.81-7.92 (m, 3H), 8.05 (s, 1H), 8.24 (d, J=7.82 Hz, 1H), 8.30 (s, 1H); Calcd for C27H23F6NO3 (M+H) 524.16, Found 524.

### Example 26

### (R*)4-Methyl-2-(4′-trifluoromethyl-5-{1-[1-(4-trifluoromethyl-phenyl)-propyl]-piperidin-3-yl}-biphenyl-3-yl)-pentanoic Acid; (R* Refers to the Stereochemistry as Draw not Determined)

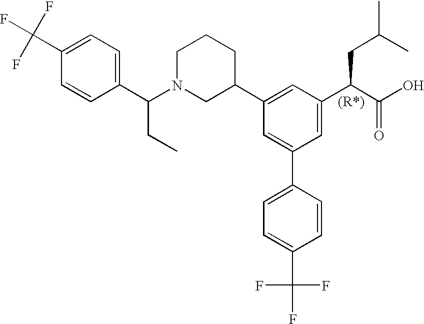

### a) 4-Methyl-2-(5-pyridin-3-yl-4′-trifluoromethyl-biphenyl-3-yl)-pentanoic Acid Ethyl Ester

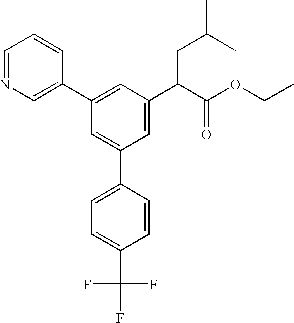

To a solution of 4-methyl-2-(5-trifluoromethanesulfonyloxy-4′-trifluoromethyl-biphenyl-3-yl)-pentanoic acid ethyl ester, prepared in Example 5 step (a) (1.26 g, 2.46 mmol) in dimethoxyethane (16 mL) was added 3-pyridine boronic acid (0.60 g, 4.9 mmol), and 2 M Na₂CO₃ (3.7 mL). The reaction was degassed, tetrakis (triphenylphosphine) palladium (0) (0.28 g, 0.35 mmol) was added, and the reaction was degassed and heated to 80° C. After 2 hours, the reaction was cooled to RT, diluted with EtOAc, washed with sat. NaHCO₃, washed with brine, dried and filtered to give the crude product. Purification via silica gel chromatography employing the Isco purification system gave the title compound (1.0 g, 92%) as a yellow oil. ¹H NMR (300 MHz, CHLOROFORM-D) δ ppm 0.96 (d, J=6.41 Hz, 6H) 1.25 (t, J=7.16 Hz, 3H) 1.49-1.59 (m, 1H) 1.75 (ddd, J=13.75, 7.16, 6.97 Hz, 1H) 2.08 (dt, J=13.56, 7.72 Hz, 1H) 3.77-3.84 (m, 1H) 4.09-4.24 (m, 2H) 7.40 (dd, J=7.54, 5.27 Hz, 1H) 7.55-7.62 (m, 2H) 7.65-7.70 (m, 1H) 7.70-7.77 (m, 4H) 7.93 (dt, J=7.91, 2.07 Hz, 1H) 8.64 (dd, J=4.90, 1.51 Hz, 1H) 8.89 (d, J=1.88 Hz, 1H); Calc'd for C₂₆H₂₆F₃NO₂ (M+H)⁺ 441.49, Found 442.3

### b) 4-Methyl-2-(5-piperidin-3-yl-4′-trifluoromethyl-biphenyl-3-yl)-pentanoic Acid Ethyl Ester

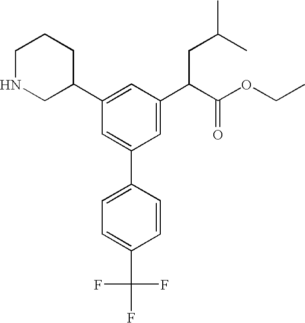

A solution of 4-methyl-2-(5-pyridin-3-yl-4′-trifluoromethyl-biphenyl-3-yl)-pentanoic acid ethyl ester, from the previous step (1.0 g, 2.3 mmol), 4 N HCl/Dioxane (0.62 mL, 2.5 mmol) in MeOH (75 mL) was hydrogenated at 40 psi with PtO₂ (51 mg, 0.1 mmol). The reaction was filtered through celite, washed with MeOH, and concentrated in vacuo. The free base was obtained by treatment with Na₂CO₃ and extraction twice into CH₂Cl₂. The organic extracts were dried, filtered and evaporated to give the title compound (977 mg, 97%) as a yellow oil. ¹H NMR (300 MHz, CHLOROFORM-D) δ ppm 0.85-0.96 (m, 6H) 1.23 (t, J=7.16 Hz, 3H) 1.49-1.56 (m, 1H) 1.58-1.74 (m, 4H) 1.81 (td, J=5.75, 2.45 Hz, 1H) 1.96-2.09 (m, 2H) 2.62-2.77 (m, 3H) 3.12 (d, J=12.43 Hz, 1H) 3.21 (d, J=8.29 Hz, 1H) 3.65-3.74 (m, 1H) 4.06-4.21 (m, 2H) 7.21 (s, 1H) 7.31 (s, 1H) 7.39 (s, 1H) 7.62-7.71 (m, 4H); Calc'd for C₂₆H₃₂F₃NO₂ (M+H)⁺ 447.53, Found 448.3

### c) 1-(4-Trifluoromethyl-phenyl)-propan-1-ol

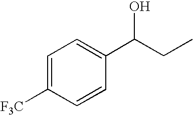

To 4-trifluoromethylpropiophenone (1.0 g, 5.0 mmol) in MeOH (25 mL, 0.20 M), was added NaBH₄ (187 mg, 5.0 mmol). After 3 hours at RT, the reaction was concentrated in vacuo, partitioned between H₂O and CH₂Cl₂, dried, filtered and concentrated to give the title compound as a white solid (0.97 g, 96%). ¹H NMR (300 MHz, CHLOROFORM-D) δ ppm 0.93 (t, J=7.54 Hz, 3H) 1.71-1.85 (m, 2H) 1.85-1.92 (m, 1H) 4.69 (td, J=6.41, 3.39 Hz, 1H) 7.44-7.50 (m, 2H) 7.61 (d, J=8.29 Hz, 2H).

### d) Methanesulfonic acid 1-(4-trifluoromethyl-phenyl)-propyl Ester

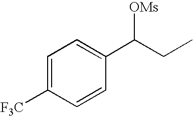

To a solution of compound 26 c from the above reaction (918 mg, 4.5 mmol) in anhydrous CH₂Cl₂ (30 mL, 0.15 M) at 0° C. was added triethylamine (2.54 mL, 18 mmol), and methanesulfonylchloride (1.0 mL, 13.5 mmol). The cold bath was removed and the reaction stirred at RT. Once complete, the reaction was quenched with 1N HCl, diluted with H₂O and extracted. The organics were washed with H₂O and brine, dried, filtered and concentrated to give the title compound as a yellow oil. ¹H NMR (300 MHz, CHLOROFORM-D) δ ppm 0.98 (t, J=7.35 Hz, 3H) 1.92 (ddd, J=13.47, 7.16, 6.88 Hz, 1H) 2.08 (dt, J=14.41, 7.30 Hz, 1H) 2.80 (s, 3H) 5.48-5.54 (m, 1H) 7.50 (d, J=8.29 Hz, 2H) 7.67 (d, J=7.91 Hz, 2H)

### e) 4-Methyl-2-(4′-trifluoromethyl-5-{1-[1-(4-trifluoromethyl-phenyl)-propyl]-piperidin-3-yl}-biphenyl-3-yl)-pentanoic Acid Ethyl Ester

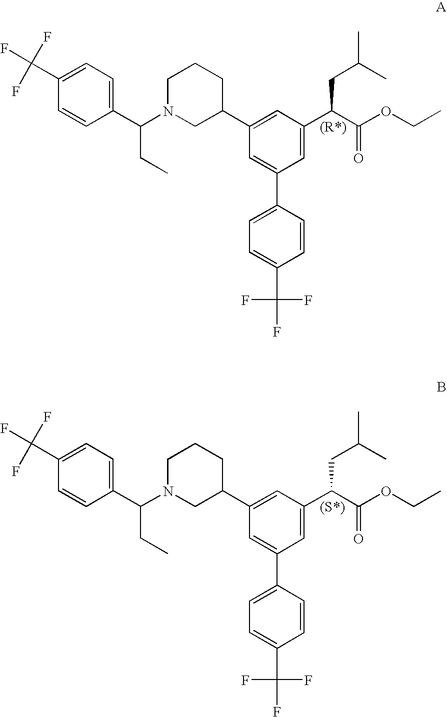

To a solution of compound 26 d obtained from the above reaction in DMF (4 mL) was added compound 26 b (1.3 g, 4.5 mmol) and Cs₂CO₃ (2.0 g, 6.0 mmol). After stirring 17 hours at RT, the reaction was poured into EtOAc, washed with NaHCO₃, H₂O (3×) and brine, dried, filtered and concentrated to give a yellow oil. Purification via silica gel chromatography employing the Isco purification system gave two mixtures of diastereomers. The stereochemistry of the alpha-chain (C-2) of two diastereomer are tentatively assigned as shown; Compound A: ¹H NMR (300 MHz, CHLOROFORM-D) δ ppm 0.80 (t, J=6.97 Hz, 3H) 0.87-0.96 (m, 6H) 1.24 (t, J=6.97 Hz, 3H) 1.43-1.67 (m, 4H) 1.95-2.10 (m, 5H) 2.40-2.60 (m, 3H) 3.35-3.60 (m, 3H) 3.69 (dd, J=8.29, 6.78 Hz, 1H) 4.05-4.21 (m, 2H) 7.15 (d, J=4.90 Hz, 1H) 7.29 (d, J=1.88 Hz, 2H) 7.47 (s, 2H) 7.57-7.76 (m, 6H); Calc'd for C₃₆H₄₁F₆NO₂ (M+H)⁺ 633.71, Found 634.3. Compound B: ¹H NMR (300 MHz, CHLOROFORM-D) δ ppm 0.73 (t, J=7.16 Hz, 1H) 0.85-0.96 (m, 7H) 1.18-1.28 (m, 3H) 1.44-2.2 (m, 9H) 2.88-2.96 (m, 4H) 3.32-3.98 (m, 4H) 4.06-4.22 (m, 2H) 7.29 (s, 1H) 7.41 (s, 1H) 7.40-7.71 (m, 9H); Calc'd for C₃₆H₄₁F₆NO₂ (M+H)⁺ 633.71, Found 634.3.

### f) (R*) 4-Methyl-2-(4′-trifluoromethyl-5-{1-[1-(4-trifluoromethyl-phenyl)-propyl]-piperidin-3-yl}-biphenyl-3-yl)-pentanoic Acid

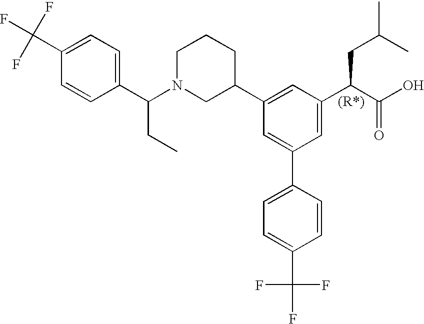

Compound A, obtained from the above reaction (125 mg, 0.197 mmol) in EtOH (10 mL) and 2M KOH (0.4 mL, 0.79 mmol) was heated to 78° C. for 2 hours, then cooled and concentrated in vacuo for 30 minutes. The concentrate was diluted with CH₂Cl₂ and H₂O; adjusting the pH to 7 with 10% citric acid, the organics were extracted 3× with CH₂Cl₂, dried and filtered. Purification via silica gel chromatography employing the Isco purification system gave the product as an oil. ¹H NMR (300 MHz, MeOD) δ ppm 0.61 (t, J=7.16 Hz, 3H) 0.79-0.89 (m, 6H) 1.44-1.71 (m, 6H) 1.91-2.20 (m, 3H) 2.82-2.43 (m, 2H) 2.75-2.89 (m, 1H) 3.09 (d, J=10.17 Hz, 1H) 3.32 (d, J=8.67 Hz, 1H) 3.53-3.64 (m, 1H) 3.88 (td, J=7.35, 3.77 Hz, 1H) 7.18-7.27 (m, 2H) 7.43-7.52 (m, 3H) 7.55-7.66 (m, 6H); Calc'd for C₃₄H₃₇F₆NO₂ (M+H)⁺ 605.65, Found 606.2. The oil was concentrated with 1N HCl/Ether to provide the title compound as the HCl salt.

### Example 27

### 4-Methyl-2-[4′-trifluoromethyl-5-(6-trifluoromethyl-piperidin-2-yl)-biphenyl-3-yl]-pentanoic Acid

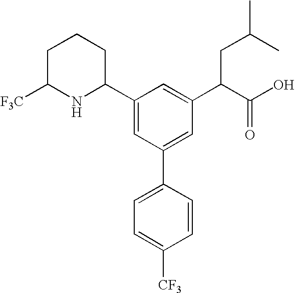

### a) 4-Methyl-2-[4′-trifluoromethyl-5-(6-trifluoromethyl-pyridin-2-yl)-biphenyl-3-yl]-pentanoic Acid Ethyl Ester

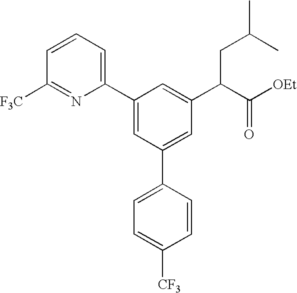

6-(Trifluoromethyl)pyridine-2-boronic acid pinacol ester was coupled with compound 5a following the same Suzuki coupling procedure as described in Example 5, step (b) to give the title compound; ¹H NMR (400 MHz, CHLOROFORM-D) δ 0.79-0.90 (m, 6H), 1.11-1.22 (m, 3H), 1.42-1.52 (m, 1H), 1.60-1.72 (m, 1H), 1.95-2.05 (m, 1H), 3.76 (t, J=7.70 Hz, 1H), 4.02-4.13 (m, 2H), 7.57-7.62 (m, 3H), 7.64-7.71 (m, 4H), 7.86-7.98 (m, 3H); Calcd for C27H25F6NO2 (M+H) 510.18, Found 510.

### b) 4-Methyl-2-[4′-trifluoromethyl-5-(6-trifluoromethyl-piperidin-2-yl)-biphenyl-3-yl]-pentanoic Acid Ethyl Ester

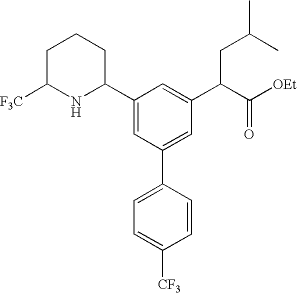

A mixture of compound 28a (970 mg, 1.9 mmol), PtO₂ (43 mg, 0.19 mmol) and 4N HCl/dioxane (0.524 mL, 2.17 mmol) in EtOH (10 mL) was hydrogenated under H₂ (20 psi) in a Parr-shaker for 1 h. The resulting reaction mixture was filtered through a celite pad and the filtrate was concentrated to give the title compound (971 mg, 99%) as a white solid;

¹H NMR (300 MHz, CHLOROFORM-D) δ 0.81-0.96 (m, 6H), 1.19-1.33 (m, 4H), 1.46-1.60 (m, 3H), 1.67 (dt, J=13.66, 6.92 Hz, 1H), 1.85-1.92 (m, 1H), 1.95 (s, 1H), 1.99-2.06 (m, 2H), 3.25-3.39 (m, 1H), 3.63-3.78 (m, 2H), 4.06-4.22 (m, 2H), 7.36 (s, 1H), 7.42-7.56 (m, 2H), 7.66-7.76 (m, 4H); Calcd for C27H31F6NO2 (M+H) 516.23, Found 516.

### c) 4-Methyl-2-[4′-trifluoromethyl-5-(6-trifluoromethyl-piperidin-2-yl)-biphenyl-3-yl]-pentanoic Acid

Compound 27a was hydrolyzed following the same hydrolysis procedure of Example 13 to give the title compound; 1H NMR (300 MHz, MeOD) δ 0.79-0.89 (m, 6H), 1.45 (dt, J=12.90, 6.55 Hz, 1H), 1.55-1.69 (m, 1H), 1.76-1.87 (m, 2H), 1.97 (dd, J=7.54, 6.03 Hz, 1H), 2.01-2.11 (m, 2H), 2.13-2.24 (m, 1H), 3.75 (t, J=7.72 Hz, 1H), 4.34 (d, J=6.78 Hz, 1H), 4.48 (dd, J=10.17, 3.77 Hz, 1H), 4.77 (s, 7H), 7.54 (s, 1H), 7.66 (s, 1H) 7.67-7.81 (m, 5H); Calcd for C25H27F6NO2 (M+H) 488.19, Found 488.1.

### Example 28

### 4-Methyl-2-{5-[1-(3-methyl-butyl)-6-trifluoromethyl-piperidin-2-yl]-4′-trifluoromethyl-biphenyl-3-yl}-pentanoic Acid

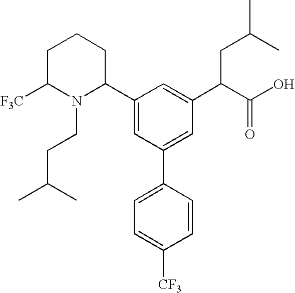

A mixture of compound 27b (240 mg, 0.465 mmol) and isovaleraldehyde (0.15 mL, 1.4 mmol) in THF (4 mL) was stirred for 1 h followed by addition of NaBH(OAc)₃ (297 mg, 1.4 mmol). The reaction mixture was stirred for 2 days and then partitioned in EtOAc and saturated NaHCO₃ solution. The organic layer was dried (Na₂SO₄), concentrated and purified by column chromatography to give the ester of the title compound, 180 mg (69%) as a white solid.

The above obtained ester was hydrolyzed following the same hydrolysis procedure as Example 13 to give the title compound; ¹H NMR (400 MHz, CHLOROFORM-D) δ 0.54-0.60 (m, 3H), 0.62-0.67 (m, 3H), 0.86-0.94 (m, 6H), 1.17-1.28 (m, 4H), 1.41-1.54 (m, 2H), 1.69-1.77 (m, 4H), 1.95-2.06 (m, 2H), 2.40-2.51 (m, 1H), 2.56-2.67 (m, 1H), 3.24 (ddd, J=9.17, 6.97, 4.16 Hz, 1H), 3.63 (dd, J=11.37, 3.06 Hz, 1H), 3.73 (td, J=7.83, 3.67 Hz, 1H), 7.34 (d, J=5.38 Hz, 1H), 7.42 (d, J=1.71 Hz, 1H), 7.53 (d, J=1.96 Hz, 1H), 7.65-7.72 (m, 4H); Calcd for C30H37F6NO2 (M+H) 558.27, Found 558.2.

### Example 29

### 4-Methyl-2-(5-{[(3-methyl-butyl)-(3,4,5-trifluoro-benzyl)-amino]-methyl}-4′-trifluoromethyl-biphenyl-3-yl)-pentanoic Acid

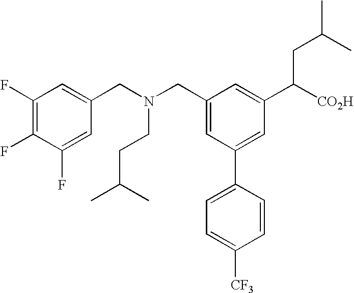

### a) 2-(5-Cyano-4′-trifluoromethyl-biphenyl-3-yl)-4-methyl-pentanoic Acid Ethyl Ester

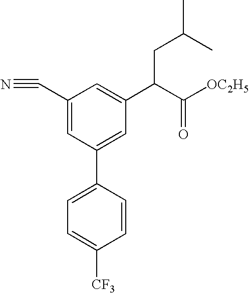

Following a literature procedure (Chackal-Catoen, S. et al. Bioorg. Med. Chem. 2006, 14, 7434), solution of compound 5a (2.18 g, 4.21 mmol) in 19.5 mL of DMF was added to a sealed tube, and zinc cyanide (1.04 g, 8.84 mmol) was added. The resulting suspension was degassed with argon for 10 min, and then tetrakis(triphenylphosphine) palladium (0) (0.49 g, 0.421 mmol) was added. The reaction flask was placed in a preheated 150° C. oil bath and was heated 24 hours. After this period, the reaction mixture was cooled and saturated aqueous NaHCO₃ solution was added. The aqueous layer was extracted with EtOAc three times. The organic extracts were combined and washed five times with brine. After drying over MgSO₄ and filtration, the resulting solution was concentrated in vacuo to provide 2.05 g of a golden brown oil. This material was purified on an ISCO chromatographic system using pure hexanes to 2:1 hexanes:ethyl acetate gradient as eluent to yield 0.93 g (57%) of 2-(5-isocyano-4′-trifluoromethyl-biphenyl-3-yl)-4-methyl-pentanoic acid ethyl ester as a colorless oil.

MH⁺ 390.3

¹H NMR (300 MHz, CDCl₃): δ0.94 (dd, J=6.6, 1.6 Hz, 6H), 1.25 (t, J=7.2 Hz, 3H), 1.42-1.55 (m, 1H), 1.63-1.76 (m, 1H), 1.96-2.10 (m, 1H), 3.75 (t, J=7.8 Hz, 1H), 4.04-4.28 (m, 2H), 7.62-7.71 (m, 3H), 7.73 (br s, 1H), 7.74-7.79 (m, 3H).

### b) 2-(5-Aminomethyl-4′-trifluoromethyl-biphenyl-3-yl)-4-methyl-pentanoic Acid Ethyl Ester

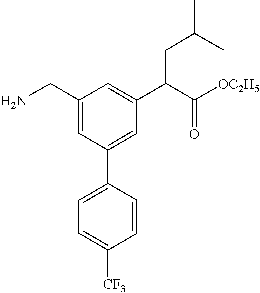

Following a literature procedure (Suh, Y.-G. et al. J. Med. Chem. 2005, 18, 7434), solution of compound 29a (0.28 g, 0.719 mmol) was dissolved in 20 mL of ethanol in a Parr hydrogenation bottle. The solution was cooled in ice, and 10% palladium on carbon (0.026 g) and concentrated (12 N) hydrochloric acid solution (0.48 mL) was added. The flask was shaken on a Parr hydrogenation apparatus at 14.5 psi for 5.25 h. After the reaction was terminated, the reaction mixture was filtered through Celite® 545 filter aid. The filtrate was concentrated in vacuo to afford a cream-colored solid. This material was dissolved in dichloromethane, and the resulting solution was washed twice with saturated aqueous Na₂CO₃ solution. After drying over Na₂SO₄ and subsequent filtration, the resulting solution was concentrated in vacuo to provide 0.28 g (quantitative yield) of 2-(5-aminomethyl-4′-trifluoromethyl-biphenyl-3-yl)-4-methyl-pentanoic acid ethyl ester as a light grey oil.

MH⁺ 394.4

¹H NMR (300 MHz, CDCl₃): δ0.93 (br d, J=6.2 Hz, 6H), 1.23 (br t, J=7.0 Hz, 3H), 1.42-1.60 (m, 1H), 1.62-1.78 (m, 1H), 1.95-2.12 (m, 1H), 2.40-2.85 (br s, 2H), 3.75 (m, 1H), 3.96 (br s, 2H), 3.96-4.23 (br m, 2H), 7.34 (br s, 1H), 7.45 (br s, 1H), 7.48 (br s, 1H), 7.68 (br s, 4H).

### c) 4-Methyl-2-{5-[(3-methyl-butylamino)-methyl]-4′-trifluoromethyl-biphenyl-3-yl}-pentanoic Acid Ethyl Ester

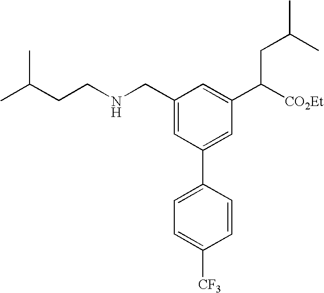

To a solution of compound 29b (0.24 g, 0.610 mmol) in 10 mL of anhydrous methanol, was added isovaleraldehyde (0.058 g, 0.07 mL, 0.671 mmol). The solution was stirred 45 minutes, and then sodium borohydride (0.046 g, 1.22 mmol) was added. After 20 h of stirring, the reaction mixture was cooled in ice, and HCl (1 N solution, 1 mL) was added. The reaction mixture was stirred for one minute, and then saturated aqueous Na₂CO₃ solution was added until the pH was basic. The solution was extracted three times with dichloromethane. The organic extracts were combined and washed with saturated aqueous Na₂CO₃ solution, dried (Na₂SO₄), and filtered. The filtrate was concentrated to provide a 0.27 g of a pale yellow glass. Purification on a flash silica gel column using 95:4.5:0.5 CH₂Cl₂:MeOH:NH₄OH provided 0.25 g (89%) of 4-methyl-2-{5-[(3-methyl-butylamino)-methyl]-4′-trifluoromethyl-biphenyl-3-yl}-pentanoic acid ethyl ester as a colorless oil.

MH⁺ 464.4

¹H NMR (300 MHz, CDCl₃): δ0.89 (d, J=6.7 Hz, 6H), 0.93 (d, J=6.6 Hz, 6H), 1.23 (t, J=7.1 Hz, 3H), 1.39-1.59 (m, 3H), 1.58-1.75 (m, 2H), 1.96-2.08 (m, 2H), 2.68 (br t, J=7.5 Hz, 2H), 3.71 (dd, J=7.3, 1.1 Hz 1H), 3.87 (s, 2H), 3.96 (br s, 2H), 4.02-4.23 (br m, 2H), 7.32 (br s, 1H), 7.45 (br d, J=1.5 Hz, 1H), 7.49 (br s, 1H), 7.69 (AB quartet, J=9.1 Hz, 4H).

### d) 4-Methyl-2-(5-{[(3-methyl-butyl)-(3,4,5-trifluoro-benzyl)-amino]-methyl}-4′-trifluoromethyl-biphenyl-3-yl)-pentanoic Acid Ethyl Ester

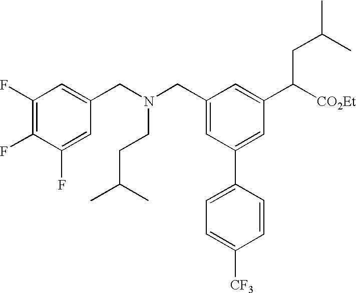

To a solution of 29c (0.037 g, 0.091 mmol) in 5 mL of anhydrous dichloromethane was added 3,4,5-trifluorobenzoaldehyde (0.029 g, 0.182 mmol). The reaction mixture was stirred 30 minutes, and then sodium triacetoxyborohydride (0.0385 g, 0.182 mmol) was added. After 18 h, 1N NaOH solution was added to the reaction mixture. The resulting mixture was extracted three times with dichloromethane. The organic extracts were combined and washed with 1N NaOH solution, dried (Na₂SO₄), filtered, and concentrated to yield a cloudy film. Purification on a flash silica gel column using 1% (5% NH₄OH in MeOH): CH₂Cl₂ yielded 0.07 g (quantitative yield) of 4-methyl-2-(5-{[(3-methyl-butyl)-(3,4,5-trifluoro-benzyl)-amino]-methyl}-4′-trifluoromethyl-biphenyl-3-yl)-pentanoic acid ethyl ester as a colorless glass.

MH⁺ 608.4

¹H NMR (300 MHz, CDCl₃): δ0.75 (d, J=6.5 Hz, 6H), 0.85 (dd, J=6.6, 1.8 Hz, 6H), 1.15 (t, J=7.1 Hz, 3H), 1.28-1.68 (m, 5H), 1.86-1.99 (m, 1H), 2.38 (br t, J=7.4 Hz, 2H), 3.40 (br s, 2H), 3.53 (br s, 2H), 3.64 (t, J=7.7 Hz 1H), 3.96-4.16 (br m, 2H), 6.92 (dd, J=8.3, 6.8 Hz, 2H), 7.28 (br s, 1H), 7.34 (br d, J=1.4 Hz, 2H), 7.61 (AB quartet, J=8.9 Hz, 4H).

### e) 4-Methyl-2-(5-{[(3-methyl-butyl)-(3,4,5-trifluoro-benzyl)-amino]-methyl}-4′-trifluoromethyl-biphenyl-3-yl)-pentanoic Acid

To a solution of 29d (0.07 g, 0.115 mmol) in 5 mL of methanol was added 3N NaOH solution (0.1 mL). The mixture was heated to 60° C. for 3 h. After cooling to ambient temperature, the reaction mixture was concentrated in vacuo. To the residue was added 3N HCl solution. This solution was extracted three times with dichloromethane. The organic extracts were combined, dried (Na₂SO₄), filtered, and concentrated to provide a milky film. Analysis by LC-MS indicated this material was a 1:2 mixture of the desired acid and the corresponding methyl ester. This material was resubjected to the reaction conditions described above for an additional 4 h and then was worked up as before to yield 0.05 g of a white foam. LC-MS indicated this material was a 1:1 mixture of the desired acid and the corresponding methyl ester. The foam was dissolved in 10 mL of methanol, and 1 mL of N NaOH solution was added. The reaction mixture was heated to 80° C. for 6 h. After cooling, the workup described before provided 0.03 g (45%) of 4-methyl-2-(5-{[(3-methyl-butyl)-(3,4,5-trifluoro-benzyl)-amino]-methyl}-4′-trifluoromethyl-biphenyl-3-yl)-pentanoic acid as a pale pink foam.

MH⁺ 580.3

¹H NMR (300 MHz, CDCl₃): δ0.73 (d, J=6.4 Hz, 6H), 0.83 (d, J=6.6 Hz, 6H), 1.32-1.48 (m, 2H), 1.54-1.70 (m, 3H), 1.83-2.00 (m, 1H), 2.59-2.96 (br s, 2H), 3.68 (t, J=7.6 Hz, 1H), 3.83-4.62 (br s, 4H), 7.28-7.45 (br s, 2H), 7.44-7.49 (br s, 1H), 7.50-7.55 (br s, 1H), 7.61 (AB quartet, J=8.3 Hz, 4H), 7.77-7.89 (br s, 1H), 12.0-12.65 (br s, 1H).

### Example 30

### 2-(5-{[(3,5-Bis-trifluoromethyl-benzyl)-(3-methyl-butyl)-amino]-methyl}-4′-trifluoromethyl-biphenyl-3-yl)-4-methyl-pentanoic Acid

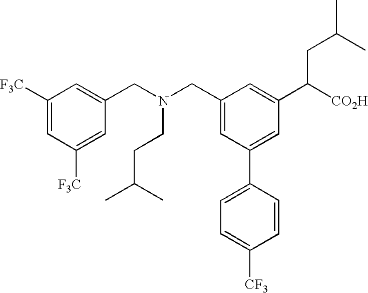

### a) 2-(5-{[(3,5-Bis-trifluoromethyl-benzyl)-(3-methyl-butyl)-amino]-methyl}-4′-trifluoromethyl-biphenyl-3-yl)-4-methyl-pentanoic Acid Ethyl Ester

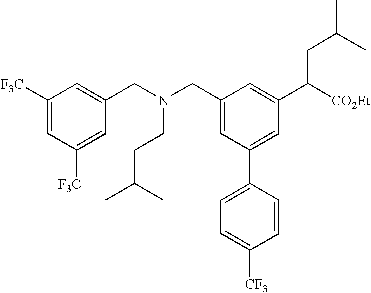

To a solution of compound 29c (0.028 g, 0.0697 mmol) in 5 mL of anhydrous dichloromethane was added 3,5-bis(trifluoromethyl)benzaldehyde (0.038 g, 0.139 mmol). The reaction mixture was stirred 30 minutes, and then sodium triacetoxyborohydride (0.0385 g, 0.182 mmol) was added. After 19 h of stirring, 1N NaOH solution was added to the reaction mixture. The resulting mixture was extracted three times with dichloromethane. The organic extracts were combined and washed with 1N NaOH solution, dried (Na₂SO₄), filtered, and concentrated to yield a cloudy film. Purification on a flash silica gel column using 99:0.5:0.5 CH₂Cl₂:MeOH:NH₄OH as the eluent yielded 0.07 g (quantitative yield) of 2-(5-{[(3,5-bis-trifluoromethyl-benzyl)-(3-methyl-butyl)-amino]-methyl}-4′-trifluoromethyl-biphenyl-3-yl)-4-methyl-pentanoic acid ethyl ester as a milky glass.

MH⁺ 690.3

¹H NMR (300 MHz, CDCl₃): δ0.73 (d, J=6.5 Hz, 6H), 0.84 (dd, J=6.6, 1.9 Hz, 6H), 1.13 (t, J=7.1 Hz, 3H), 1.12-1.28 (m, 1H), 1.31-1.48 (m, 2H), 1.48-1.68 (m, 2H), 1.86-2.03 (m, 1H), 2.42 (br t, J=7.3 Hz, 2H), 3.56 (s, 2H), 3.60 (s, 2H), 3.51-3.68 (m, 1H), 3.94-4.18 (br m, 2H), 7.27 (br s, 1H), 7.36 (br s, 1H), 7.37 (br s, 1H), 7.60 (AB quartet, J=8.9 Hz, 4H), 7.62 (br s, 1H), 7.78 (br s, 2H).

### b) 2-(5-{[(3,5-Bis-trifluoromethyl-benzyl)-(3-methyl-butyl)-amino]-methyl}-4′-trifluoromethyl-biphenyl-3-yl)-4-methyl-pentanoic Acid

To a solution of compound 30a (0.07 g, 0.115 mmol) in 5 mL of methanol was added 3N NaOH solution (0.06 mL). The mixture was heated to 55° C. for 4 h. After cooling to ambient temperature, the reaction mixture was concentrated in vacuo. To the residue was added 3N HCl solution, and the resulting solution was extracted three times with dichloromethane. The organic extracts were combined, dried (Na₂SO₄), filtered, and concentrated to provide a white foam. Analysis by LC-MS indicated this material was a 3:1 mixture of the desired acid and the corresponding methyl ester. This material was dissolved in MeOH (1 mL), and 3N NaOH solution (0.06 mL) was added. The mixture was heated to 55° C. for an additional 4 h and then was worked up as before to yield 0.06 g (79%) of 2-(5-{[(3,5-bis-trifluoromethyl-benzyl)-(3-methyl-butyl)-amino]-methyl}-4′-trifluoromethyl-biphenyl-3-yl)-4-methyl-pentanoic acid as a white foam.

MH⁺ 662.4

¹H NMR (300 MHz, CDCl₃): δ0.73 (d, J=6.3 Hz, 6H), 0.82 (d, J=6.5 Hz, 6H), 1.33-1.48 (m, 2H), 1.53-1.71 (m, 3H), 1.84-2.03 (m, 1H), 2.63-2.80 (br s, 2H), 3.70 (t, J=7.7 Hz, 1H), 3.65-4.38 (br s, 4H), 7.32-7.45 (br s, 1H), 7.45-7.52 (br s, 1H), 7.52 (br s, 1H), 7.61 (AB quartet, J=8.3 Hz, 4H), 7.75-7.85 (br s, 2H), 8.02-8.32 (br s, 2H), 12.20-12.82 (br s, 1H).

### Example 31

### 4-Methyl-2-{4′-trifluoromethyl-5-[1-(4-trifluoromethyl-benzyl)-piperidin-4-yl]-biphenyl-3-yl}-pentanoic Acid

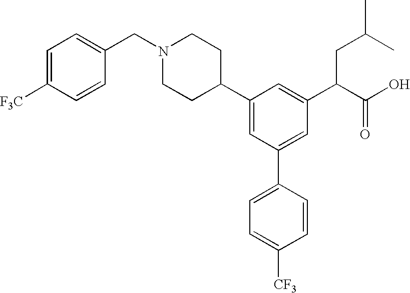

### a) Ethyl-2-(5-pyridin-4-yl-4′-trifluoromethyl-biphenyl-3-yl)-pentanoic Acid Ethyl Ester

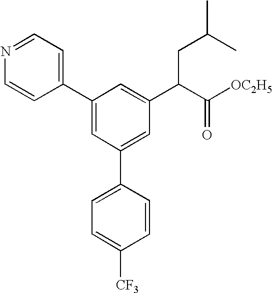

A mixture of 4-methyl-2-(5-trifluoromethanesulfonyloxy-4′-trifluoromethyl-biphenyl-3-yl)-pentanoic acid ethyl ester, compound 5a (2.0 g, 3.90 mol), pyridine-4-boronic acid (540 mg, 4.39 mol), 1,2-dimethoxyethane (8 mL) and aqueous Na₂CO₃ (2 M, 1.93 mL, 3.90 mol) was mechanically stirred while purging with N₂ at room temperature for 10 min. To this system was added Pd(PPh₃)₄ (75 mg, 0.06 mmol) and heated to reflux (95° C.) for 2 h. Another portion of Pd(PPh₃)₄ (75 mg, 0.06 mmol) was added and the reaction was heated to reflux (95° C.) for an additional 2 h. The red-brown mixture was diluted with EtOAc (25 mL) and washed with saturated aqueous NaHCO₃ solution (3×50 mL) and brine (2×50 mL). The organic fraction was dried (Na₂SO₄) and concentrated in vacuo. The crude mixture was purified by ISCO column chromatography to obtain 4-methyl-2-(5-pyridin-4-yl-4′-trifluoromethyl-biphenyl-3-yl)-pentanoic acid ethyl ester.

¹H-NMR (CDCl₃): δ 0.95 (d, 6H), 1.25 (t, 3H), 1.56 (m, 1H), 1.75 (m, 1H), 2.06 (m, 1H), 3.80 (t, 1H), 4.15 (m, 2H), 7.56 (dd, 2H), 7.63 (d, 2H), 7.73 (m, 5H), 8.70 (dd, 2H); Calcd for C26H26F3NO2 (M+H) 442.49, Found 442.67

### b) Ethyl-2-(5-piperidin-4-yl-4′-trifluoromethyl-biphenyl-3-yl)-pentanoic Acid Ethyl Ester

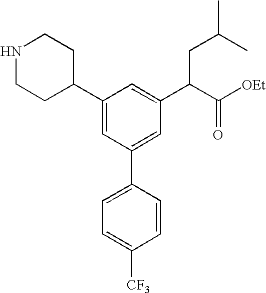

To a solution of 4-methyl-2-(5-pyridin-4-yl-4′-trifluoromethyl-biphenyl-3-yl)-pentanoic acid ethyl ester, compound 31a (1.15 g, 2.61 mmol) in MeOH (50 mL) was added 4N HCl (0.717 mL, 2.88 mmol) and the mixture was allowed to stand for 5 minutes. To this solution was added PtO₂ (25 mg). The suspension was stirred for 10 minutes and filtered through celite. To the filtrate was added another portion of PtO₂ (25 mg). The black suspension was hydrogenated at 40 psi overnight. The suspension was re-filtered. To the filtrate was added another portion of PtO₂ (25 mg) and 4N HCl (0.100 mL). The black suspension was hydrogenated at 40 psi for 2 days. The mixture was filtered through celite and the solvent was removed in vacuo. The crude mixture was purified by ISCO column chromatography to obtain a mixture of 4-methyl-2-(5-piperidin-4-yl-4′-trifluoromethyl-biphenyl-3-yl)-pentanoic acid methyl ester and 4-methyl-2-(5-piperidin-4-yl-4′-trifluoromethyl-biphenyl-3-yl)-pentanoic acid ethyl ester. Calcd for C25H30F3NO2 (M+H) 434.51, Found 434.23 (methyl ester) and Calcd for C26H32F3NO2 (M+H) 448.53, Found 448.43 (ethyl ester).

### c) Ethyl-2-{4′-trifluoromethyl-5-[1-(4-trifluoromethyl-benzyl)-piperidin-4-yl]-biphenyl-3-yl}-pentanoic Acid Ethyl Ester

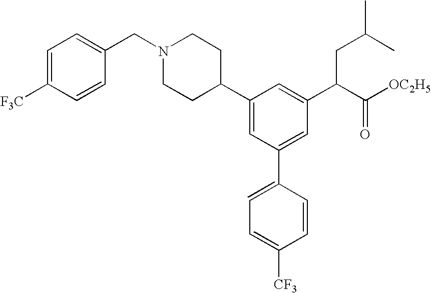

To a solution of a mixture of 4-methyl-2-{5-piperidin-4-yl-4′-trifluoromethyl-biphenyl-3-yl)-pentanoic acid ethyl ester and methyl ester, compound 31b (57 mg, 0.13 mmol) in 1,2-dichloroethane (1.0 mL) was added 4-trifluoromethyl-benzaldehyde (0.019 mL, 0.14 mmol) and sodiumtriacetoxyborohydride (35 mg, 0.17 mmol). The mixture was stirred for 2 h at room temperature. The reaction was quenched with water and extracted with dichloromethane. The organic was washed with saturated aqueous NaHCO₃ solution and brine. The organic fraction was dried (MgSO₄) and concentrated in vacuo. The crude mixture was purified by ISCO column chromatography to obtain a mixture of 4-methyl-2-{4′-trifluoromethyl-5-[1-(4-trifluoromethyl-benzyl)-piperidin-4-yl]-biphenyl-3-yl}-pentanoic acid ethyl ester and methyl ester. Calcd for C33H35F6NO2 (M+H) 592.63, Found 592.30 (methyl ester) and Calcd for C34H37F6NO2 (M+H) 606.65, Found 606.30 (ethyl ester).

### d) 4-Methyl-2-{4′-trifluoromethyl-5-[1-(4-trifluoromethyl-benzyl)-piperidin-4-yl]-biphenyl-3-yl}-pentanoic Acid

To a solution of a mixture of 4-methyl-2-{4′-trifluoromethyl-5-[1-(4-trifluoromethyl-benzyl)-piperidin-4-yl]-biphenyl-3-yl}-pentanoic acid ethyl ester and methyl ester, compound 31c (56 mg, 0.09 mmol) in MeOH (1 mL) was added 3N NaOH (0.060 mL) and heated to 50° C. for 2 h. The reaction was concentrated in vacuo to remove MeOH. The thick liquid was acidified to pH 2 with 2N HCl. The resulting acidic solution was extracted with EtOAc. The organic fraction was dried (MgSO₄) and concentrated in vacuo. The crude mixture was purified by ISCO column chromatography to obtain 4-methyl-2-{4′-trifluoromethyl-5-[1-(4-trifluoromethyl-benzyl)-piperidin-4-yl]-biphenyl-3-yl}-pentanoic acid.

¹H-NMR (DMSO-d₆): δ 0.88 (d, 6H), 1.42 (m, 1H), 1.60 (m, 1H), 1.92 (m, 1H), 2.01 (bs, 4H), 2.80-3.51 (m, 5H), 3.69 (t, 1H), 4.10 (bs, 1H), 4.47 (bs, 1H), 7.26 (s, 1H), 7.45 (s, 1H), 7.52 (s, 1H), 7.86 (m, 8H); Calcd for C32H33F6NO2 (M+H) 578.60, Found 578.

### Example 32

### 4-Methyl-2-(5-{1-[4-methyl-1-(4-trifluoromethyl-phenyl)-pentyl]-piperidin-4-yl}-4′-trifluoromethyl-biphenyl-3-yl)-pentanoic Acid

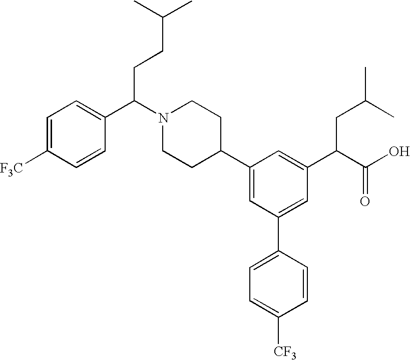

### a) 4-Methyl-2-(5-{1-[4-methyl-1-(4-trifluoromethyl-phenyl)-pentyl]-piperidin-4-yl}-4′-trifluoromethyl-biphenyl-3-yl)-pentanoic Acid Ethyl Ester

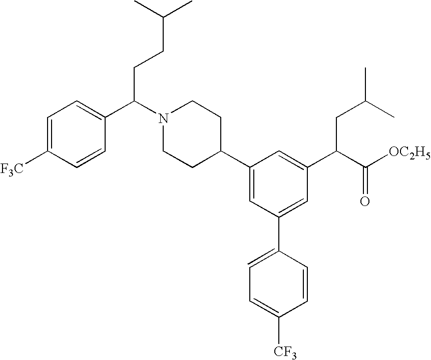

To a solution of a mixture of 4-methyl-2-(5-piperidin-4-yl-4′-trifluoromethyl-biphenyl-3-yl)-pentanoic acid ethyl ester and methyl ester 31b (54 mg, 0.12 mmol) in toluene (1.0 mL) was added 4-trifluoromethyl-benzaldehyde (0.017 mL, 0.13 mmol) and 1H-benzotriazole (16 mg, 0.13 mmol). The mixture was heated at 130° C. for 18 h under Dean-Stark condition. The reaction mixture was concentrated in vacuo and dried in the pump for 4 h. The resulting thick yellow oil was dissolved in dichloromethane (1 mL) and cooled to 8° C. To this cold solution was added 3-methylbutylzincbromide (0.5 M in THF, 0.720 mL, 0.36 mmol) drop-wise maintaining the internal temperature below 10° C. The resulting solution was stirred at 10° C. for 1 h and at room temperature for 24 h. To this incomplete reaction mixture was added another equivalent of 3-methylbutylzincbromide (0.5 M in THF, 0.240 mL, 0.12 mmol) and stirred for 2 more days. The reaction was quenched with saturated aqueous NH₄Cl solution and diluted with dichloromethane. The organic layer was washed with H₂O, dried over (MgSO₄) and concentrated in vacuo. The crude mixture was purified by ISCO column chromatography to obtain a mixture of 4-methyl-2-(5-{1-[4-methyl-1-(4-trifluoromethyl-phenyl)-pentyl]-piperidin-4-yl}-4′-trifluoromethyl-biphenyl-3-yl)-pentanoic acid ethyl ester and methyl ester. Calcd for C38H45F6NO2 (M+H) 662.76, Found 662.4 (methyl ester) and Calcd for C39H47F6NO2 (M+H) 676.79, Found 676.79 (ethyl ester).

### b) 4-Methyl-2-(5-{1-[4-methyl-1-(4-trifluoromethyl-phenyl)-pentyl]-piperidin-4-yl}-4′-trifluoromethyl-biphenyl-3-yl)-pentanoic Acid

To a solution of a mixture of 4-methyl-2-(5-{1-[4-methyl-1-(4-trifluoromethyl-phenyl)-pentyl]-piperidin-4-yl}-4′-trifluoromethyl-biphenyl-3-yl)-pentanoic acid ethyl ester and methyl ester from the previous step (56 mg, 0.09 mmol) in MeOH (1 mL) was added 3N NaOH (0.060 mL) and heated to 50° C. for 2 h. The reaction was concentrated in vacuo to remove MeOH. The thick liquid was acidified to pH 2 using 2N HCl. The resulting acidic solution was extracted with EtOAc. The organic fraction was dried (MgSO₄) and concentrated in vacuo. The crude mixture was purified by ISCO column chromatography to obtain 4-methyl-2-(5-{1-[4-methyl-1-(4-trifluoromethyl-phenyl)-pentyl]-piperidin-4-yl}-4′-trifluoromethyl-biphenyl-3-yl)-pentanoic acid.

¹H-NMR (DMSO-d₆): δ 0.78 (m, 1H), 0.86 (d, 6H), 0.88 (d, 6H), 1.00 (m, 1H), 1.42 (m, 1H), 1.57 (m, 2H), 1.83-2.10 (m, 5H), 2.22 (bs, 2H), 2.85 (bs, 4H), 3.70 (t, 1H), 3.81 (bd, 1H), 4.57 (bs, 1H), 7.22 (s, 1H), 7.41 (s, 1H), 7.53 (s, 1H), 7.78-7.99 (m, 8H), 9.82 (s, 1H); Calcd for C37H43F6NO2 (M+H) 648.73, Found 648.5

### Example 33

### 2-[5-(4-tert-Butyl-cyclohexylamino)-4′-trifluoromethyl-biphenyl-3-yl]-4-methyl-pentanoic Acid

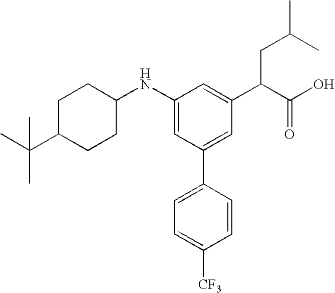

### a) 2-[5-(4-tert-Butyl-cyclohexylamino)-4′-trifluoromethyl-biphenyl-3-yl]-4-methyl-pentanoic Acid Ethyl Ester

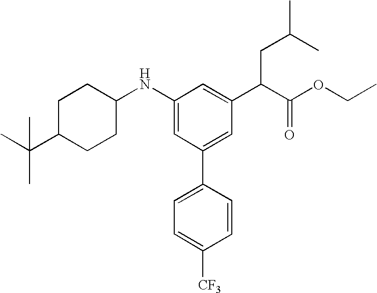

A mixture of 4-t-butylcyclohexylamine (0.17 mL, 0.95 mmol), compound 5a (0.24 g, 0.46 mmol), Pd₂(dba)₃ (0.04 g), dppbiphenyl (0.015 g), potassium phosphate tribasic (0.12 g, 0.6 mmol), DME (2 mL) was placed in a sealed reaction tube and heated to 100° C. for 10 min. LC/MS indicated 50% conversion. The solid was removed by filtration, and the solvent was removed by vacuum. The crude product was purified by Gilson HPLC to give 46 mg product. (20% yield), MH⁺ 518.4

### b) 2-[5-(4-tert-Butyl-cyclohexylamino)-4′-trifluoromethyl-biphenyl-3-yl]-4-methyl-pentanoic Acid

A solution of compound 33a (0.016 g) in MeOH/THF/NaOH (5 mL/5 ml/1N 0.5 ml) was stirred overnight. After Gilson HPLC purification, the TFA salt was converted to the Na salt, to give 13 mg of the title product. (80% yield). ¹H NMR (300 MHz, CD₃OD): δ0.8 (s, 9H), δ0.85 (m, 6H), δ1.1-1.8 (m, 10H), δ2.1 (m, 2H), δ3.21 (m, 1H), δ3.5 (m, 1H), δ6.56 (d, 1H, J=1.8 Hz), δ6.68 (t, 1H, J=1.8 Hz), δ6.73 (s, 1H), δ7.62 (m, 4H). MH⁺ 490.4

### Example 34

### 2-{5-[1-(3,5-Difluoro-phenyl)-4-methyl-pentylamino]-4′-trifluoromethyl-biphenyl-3-yl}-4-methyl-pentanoic Acid

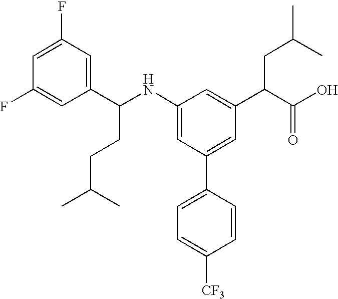

### a) 3-methyl-1-butyl Magnesium Bromide

To a dry 100 ml three-neck flask equipped with a dry ice condenser under N₂ was added Mg turnings (1.5 g, 0.0625 mol) HgCl₂ (0.1 g), ether (60 ml) and 1-Br-3-methylbutane (8 g, 0.053 mol). The resulting mixture was stirred at room temperature 20 min and then heated to reflux for 30 min. The obtained Grignard reagent was used for step (c).

### b) 3,5-Difluoro-N-methoxy-N-methyl-benzamide

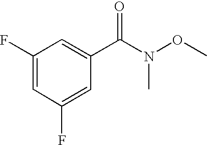

To an ice cooled mixture of 3,5 difluoro benzoic acid (2.0 g, 0.012 mol), 1-hydroxy-benzotriazole (HOBT, 2.5 g, 0.018 mol) and N,O-dimethylhydroxylamine HCl (2.35 g, 0.025 mol) in CH₂Cl₂ (100 mL) was added triethylamine (5.0 mL, 0.036 mol) and 1,3-dimethylamino propyl-3-ethylcarbodiimide (EDC, 3.8 g, 0.019 mol). The mixture was allowed to warm to room temperature and continuously stir overnight. The reaction mixture was added EtOAc (300 mL) and then washed by diluted HCl solution, NaHCO₃ and NaCl solution. The organic layer was collected and dried with Mg₂SO₄ and evaporated. The crude product was purified by column (0-50% EtOAc/Heptane) to give the title compound, 2.6 gm colorless oil. (100%)

c) 1-(3,5-Difluoro-phenyl)-4-methyl-pentan-1-one

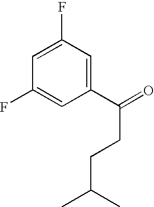

To a stirred solution of compound 34b (2.6 g, 0.012 mol) in THF (50 mL) at 0° C. was added the Grignard solution prepared in step (a) (30 mL, 0.026 mol) dropwise. After the addition, the reaction solution was stirred at room temperature for 20 min, followed by the addition of EtOAc (100 mL), and aqueous NaHCO₃. The EtOAc layer was collected and washed with aqueous NaCl. The organic layer was concentrated and the crude product purified by column (0-30% EtOAc/heptane) to give the title compound, 2.16 g, as a colorless oil. (79%).

### d) 1-(3,5-Difluoro-phenyl)-4-methyl-pentylamine

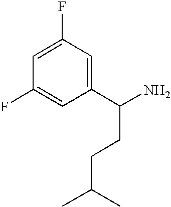

To a sodium ethoxide solution, prepared from sodium (0.26 g, 0.011 mol) in ethanol (10 mL), was added a solution of hydroxylamine hydrochloride (0.785 g, 0.011 mol) in water (5 mL). The resulting solution was stirred at room temperature for 30 min. The precipitate was filtered off and washed with alcohol (5 mL). To the combined filtrate was added compound 34c (2.16 g, 0.01 mol) and heated to reflux for 1 hour. The reaction mixture was diluted with EtOAc (100 mL) and then was washed with NaCl aq. The EtOAc layer was dried over Mg₂SO₄, filtered, and concentrated. The crude hydroxyl imine was then placed in a hydrogenation bottle with MeOH (30 mL), NH₄OH (1 mL) and Pd—C 10% (0.2 g) and subjected to hydrogenation under 5 psi for two hrs. The catalyst was filtered out and MeOH was removed by vacuum to give the title compound as an oil, 2.0 g (88% yield at 95% purity). MH⁺ 214.2

### e) 2-{5-[1-(3,5-Difluoro-phenyl)-4-methyl-pentylamino]-4′-trifluoromethyl-biphenyl-3-yl}-4-methyl-pentanoic Acid Ethyl Ester

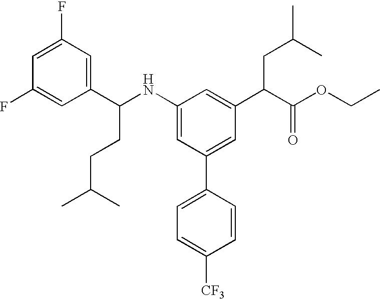

A solution of compound 34d (0.5 g, 2 mmol), triflate from compound 5a (0.5 g, 1 mmol), Pd₂(dba)₃ (0.09 g), dppbiphenyl (0.03 g), potassium phosphate tribasic (0.25 g, 1.2 mmol), DME (10 mL) in a microwave reaction tube was subjected to microwave irradiation (100° C., 20 min). The solvent was removed by vacuum and the crude product was purified by column to give the title compound, 0.35 gm oil (62%). MH⁺ 576.3

### f) 2-{5-[1-(3,5-Difluoro-phenyl)-4-methyl-pentylamino]-4′-trifluoromethyl-biphenyl-3-yl}-4-methyl-pentanoic Acid

A solution of compound 34e (0.08 g), 1N NaOH (1 mL) in THF/MeOH (10 mL/10 mL) was stirred at room temperature for two days. EtOAc (50 mL) added. The organic layer was washed with citric acid aq., NaCl aq., dried with Mg₂SO₄, and evaporated. The crude product was purified by prep TLC (40% EtOAc/Heptane) to give the title compound, 0.012 g acid product (16%).

MH⁺ 648.3

¹H NMR (300 MHz, CD₃OD): δ0.66 (m, 3H), δ0.74 (m, 3H), δ0.81 (m, 6H), δ1.2-1.8 (m, 8H), δ3.4 (m, 1H), δ4.5 (m, 1H), δ6.4 (m, 1H), δ6.7 (m, 2H), δ7.4 (s, 4H), δ7.7 (s, 1H), δ7.9 (s, 2H).

### Example 35

### (S*)4-Methyl-2-(4′-trifluoromethyl-5-{1-[1-(4-trifluoromethyl-phenyl)-propyl]-piperidin-3-yl}-biphenyl-3-yl)-pentanoic Acid

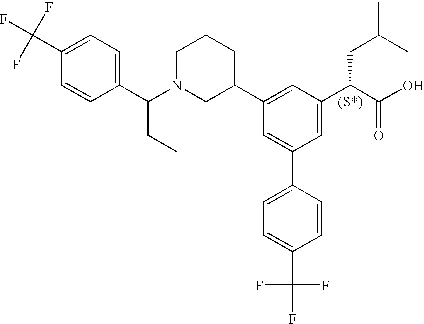

Compound B, prepared from Example 26, step (e) (200 mg, 0.316 mmol) in EtOH (15 mL) and 2M KOH (0.6 mL, 1.26 mmol) was heated to 78° C. for 2 hours, cooled and concentrated in vacuo for 30 minutes. The concentrate was diluted with CH₂Cl₂ and H₂O; adjusted to pH 7 with 10% citric acid, and the organics were extracted 3× with CH₂Cl₂, dried and filtered. Purification via silica gel chromatography employing the Isco purification system gave the product as an oil. ¹H NMR (300 MHz, MeOD) δ ppm 0.61 (t, J=7.35 Hz, 3H) 0.82-0.90 (m, 6H) 1.45-1.61 (m, 6H) 1.91-2.03 (m, 2H) 2.06-2.13 (m, 1H) 2.25-2.39 (m, 2H) 2.79-2.90 (m, 1H) 3.07 (d, J=11.30 Hz, 1H) 3.34 (d, J=10.17 Hz, 1H) 3.53-3.66 (m, 1H) 3.78-3.89 (m, 1H) 7.20-7.28 (m, 2H) 7.45-7.53 (m, 3H) 7.57-7.68 (m, 6H);

Calc'd for C34H₃₇F₆NO₂ (M+H)⁺ 605.65, Found 606.2.

The oil was concentrated with 1N HCl/Ether to provide the title compound as the HCl salt.

### Example 36

### 4-Methyl-2-{4′-trifluoromethyl-5-[1-(4-trifluoromethyl-benzyl)-piperidin-3-yl]-biphenyl-3-yl}-pentanoic Acid

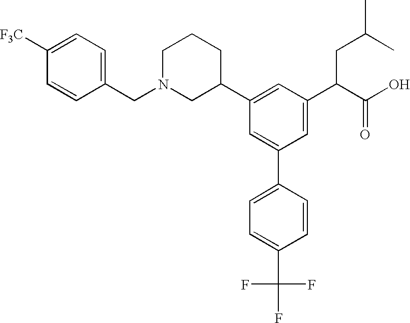

### a) 4-Methyl-2-{4′-trifluoromethyl-5-[1-(4-trifluoromethyl-benzyl)-piperidin-3-yl]-biphenyl-3-yl}-pentanoic Acid Ethyl Ester

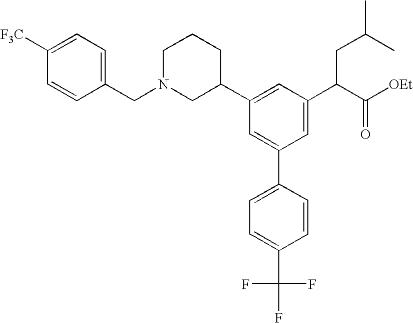

Compound 26b (357 mg, 0.800 mmol), 4-(trifluoromethyl)benzaldehyde (146 mg, 0.838 mmol), and benzotriazole (100 mg. 0.838 mmol) in toluene (4 mL) were combined and heated to reflux in a dean stark condenser for 22 hours. The reaction mixture solvent was carefully removed on a rotary evaporator and the residue was re-dissolved in THF and cooled to −10° C. To the cold stirred solution was added 3-methyl butyl zinc bromide (4.8 mL, 0.1M in THF obtained from Aldrich) dropwise. The reaction mixture was allowed to stir in the cold bath for 1 h, at room temperature overnight, and was quenched with sat. NH₄Cl solution. The mixture was diluted with CH₂Cl₂/H₂O and was filtered through a celite pad, extracted with CH₂Cl₂ (3×), dried, filtered and concentrated in vacuo. The residue was purified via silica gel chromatography employing the Isco purification system to give the title compound as a brown oil. (Note: The reaction did not give the desired product, compound 37a shown in Example 37, instead the reductive amination product with 4-(trifluomethylbenzaldehyde. It was possible due to the bad zinc bromide reagent obtained from Aldrich). ¹H NMR (300 MHz, CHLOROFORM-D) δ ppm 0.92 (d, J=6.78 Hz, 6H) 1.18-1.25 (m, 3H) 1.51-1.54 (m, 2H) 1.60-1.70 (m, 3H) 1.78-2.16 (m, 4H) 2.85-3.00 (m, 3H) 3.59 (s, 2H) 3.68 (dd, J=8.48, 6.97 Hz, 1H) 4.05-4.20 (m, 2H) 7.21 (s, 1H) 7.29-7.33 (m, 1H) 7.36-7.50 (m, 3H) 7.52-7.59 (m, 2H) 7.60-7.72 (m, 4H) Calc'd for C₃₄H₃₇F₆NO₂ (M+H)⁺ 605.65, Found 606.3

### b) 4-Methyl-2-{4′-trifluoromethyl-5-[1-(4-trifluoromethyl-benzyl)-piperidin-3-yl]-biphenyl-3-yl}-pentanoic Acid

Compound 36a (67 mg, 0.111 mmol) in EtOH (5.5 mL) and 2M KOH (0.6 mL, 1.1 mmol) was heated to reflux for 2 hours, cooled, and concentrated in vacuo for 30 minutes. The concentrate was diluted with CH₂Cl₂ and H₂O; adjusted to pH 7 with 10% citric acid, and the organics were extracted 3× with CH₂Cl₂, dried and filtered. Purification via silica gel chromatography employing the Isco purification system followed by lyophilization gave the title compound. ¹H NMR (300 MHz, MeOD) δ ppm 0.96 (d, J=6.41 Hz, 6H) 1.50-1.81 (m, 4H) 1.88-2.07 (m, 3H) 2.44-2.63 (m, 2H) 2.95-3.03 (m, 1H) 3.13-3.25 (m, 2H) 3.66-3.74 (m, 1H) 3.89-4.02 (m, 2H) 7.33 (d, J=3.77 Hz, 1H) 7.41 (s, 1H) 7.54 (s, 1H) 7.61-7.76 (m, 6H) 7.78-7.82 (m, 2H). Calc'd for C₃₂H₃₃F₆NO₂ (M+H)⁺ 577.60, Found 578.3.

### Example 37

### (R*)4-Methyl-2-(5-{1-[4-methyl-1-(4-trifluoromethyl-phenyl)-pentyl]-piperidin-3-yl}-4′-trifluoromethyl-biphenyl-3-yl)-pentanoic Acid

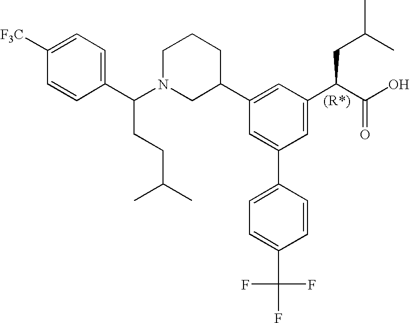

### a) 4-Methyl-2-(5-{1-[4-methyl-1-(4-trifluoromethyl-phenyl)-pentyl]-piperidin-3-yl}-4′-trifluoromethyl-biphenyl-3-yl)-pentanoic Acid Ethyl Ester

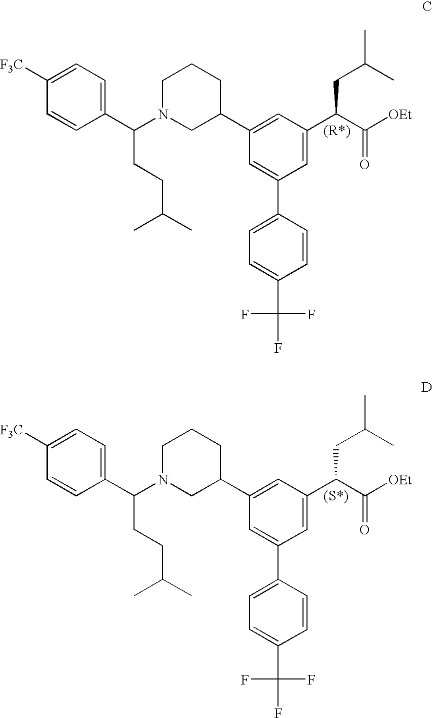

Compound 26b (341 mg, 0.762 mmol), 4-(trifluoromethyl)benzaldehyde (107 μL, 0.800 mmol), and benzotriazole (95.3 mg. 0.800 mmol) in toluene (4 mL) were combined and heated at reflux in a dean stark condenser for 18 hours. The cooled residue was concentrated, and pumped for several hours. The residue was dissolved in CH₂Cl₂ (8.0 mL, 0.1M), cooled to an internal temperature of <10° C., and 3-methylbutylzinc (4.6 mL, 2.3 mmol) was added while maintaining the <10° C. temperature. After 45 minutes, the bath was removed and the reaction continued at RT overnight. The reaction mixture was cooled to 0° C., quenched with sat. NH₄Cl (5.6 mL) and then stirred for 30 minutes before being diluted with CH₂Cl₂/H₂O. The solution was filtered through a pad of celite, extracted with CH₂Cl₂ (3×), dried, filtered and concentrated in vacuo. Purification via silica gel chromatography employing the Isco purification system gave the compounds as two disatereomers. The stereochemistry of the alpha-side chain of the two diastereomers are tentatively assigned as shown C(R*) and D (S*).

Compound C: ¹H NMR (400 MHz, CHLOROFORM-D) δ ppm 0.82 (dd, J=6.60, 3.18 Hz, 6H) 0.93 (d, J=6.60 Hz, 6H) 1.19-1.28 (m, 3H) 1.43-1.53 (m, 2H) 1.60-1.68 (m, 2H) 1.71-1.79 (m, 2H) 1.81-2.05 (m, 9H) 2.85-2.94 (m, 1H) 3.05-3.08 (m, 1H) 3.40 (dd, J=9.17, 5.26 Hz, 1H) 3.65-3.71 (m, 1H) 4.05-4.21 (m, 2H) 7.19 (s, 1H) 7.29-7.34 (m, 3H) 7.39 (s, 1H) 7.56 (d, J=8.31 Hz, 2H) 7.64-7.70 (m, 4H). Calc'd for C₃₉H₄₇F₆NO₂ (M+H)⁺ 675.79, Found 676.5.

Compound D: ¹H NMR (400 MHz, CHLOROFORM-D) δ ppm 0.83 (dd, J=6.60, 2.69 Hz, 6H) 0.87-0.94 (m, 6H) 1.21 (t, J=7.21 Hz, 3H) 1.36-1.52 (m, 4H) 1.64 (dt, J=13.69, 6.85 Hz, 1H) 1.70-1.82 (m, 4H) 1.87-1.96 (m, 3H) 1.98-2.05 (m, 2H) 2.76-2.81 (m, 1H) 2.91-2.99 (m, 2H) 3.44 (dd, J=9.05, 5.14 Hz, 1H) 3.64-3.69 (m, 1H) 4.05-4.18 (m, 2H) 7.15 (s, 1H) 7.29-7.38 (m, 4H) 7.55 (d, J=8.07 Hz, 2H) 7.62-7.69 (m, 4H). Calc'd for C₃₉H₄₇F₆NO₂ (M+H)⁺ 675.79, Found 676.5.

### b) (R*)4-Methyl-2-(5-{1-[4-methyl-1-(4-trifluoromethyl-phenyl)-pentyl]-piperidin-3-yl}-4′-trifluoromethyl-biphenyl-3-yl)-pentanoic Acid

A mixture of Compound C, (67 mg, 0.099 mmol) in EtOH (5.5 mL) and 2M KOH (0.5 mL, 0.99 mmol) was heated to reflux for 3 hours, cooled, and concentrated in vacuo. Purification via Gilson Preparative HPLC, subsequent salt exchange with 1N HCl, followed by lyophilization gave the title compound as the HCl salt.

¹H NMR (400 MHz, MeOD) δ ppm 0.50-0.61 (m, 1H) 0.64 (dd, J=6.60, 1.22 Hz, 6H) 0.73 (d, J=6.36 Hz, 6H) 0.83-0.93 (m, 1H) 1.27-1.39 (m, 2H) 1.41-1.48 (m, 2H) 1.59-1.90 (m, 4H) 2.05-2.14 (m, 2H) 2.6-2.8 (m, 2H) 3.00-3.10 (m, 1H) 3.28-3.31 (m, 1H) 3.54 (t, J=7.70 Hz, 2H) 3.60 (d, J=11.9 Hz, 1H) 4.21-4.24 (m, 1H) 7.09 (s, 1H) 7.28 (s, 1H) 7.35 (s, 1H) 7.51-7.59 (m, 5H) 7.63 (d, J=8.07 Hz, 2H). Calc'd for C₃₇H₄₃F₆NO₂ (M+H)⁺ 647.32, Found 648.5.

### Example 38

### (S*)4-Methyl-2-(5-{1-[4-methyl-1-(4-trifluoromethyl-phenyl)-pentyl]-piperidin-3-yl}-4′-trifluoromethyl-biphenyl-3-yl)-pentanoic Acid

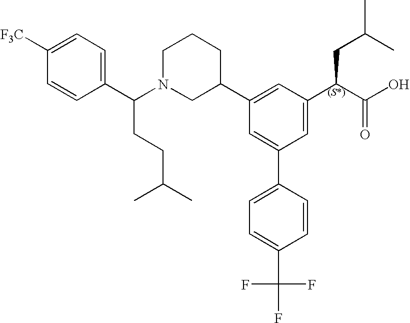

A mixture of Compound D, prepared in Example 37, step (a) (75.4 mg, 0.112 mmol) in EtOH (5.6 mL) and 2M KOH (0.6 mL, 1.12 mmol) was heated to reflux for 3 hours, cooled, and concentrated in vacuo. Purification via Gilson Preparative HPLC, subsequent salt exchange with 1N HCl, followed by lyophilization gave the title compound as the HCl salt.

¹H NMR (400 MHz, MeOD) δ ppm 0.74-0.82 (m, 1H) 0.86 (dd, J=6.60, 3.42 Hz, 7H) 0.90-0.95 (m, 6H) 1.06-1.16 (m, 1H) 1.47 (dt, J=13.39, 6.63 Hz, 1H) 1.58 (ddd, J=13.08, 6.72, 6.60 Hz, 1H) 1.84 (s, 1H) 1.93-2.05 (m, 3H) 2.08 (s, 2H) 2.30 (dd, J=10.52, 5.14 Hz, 2H) 2.91-3.02 (m, 2H) 3.12-3.21 (m, 1H) 3.45-3.52 (m, 1H) 3.74 (t, J=7.83 Hz, 1H) 3.82 (s, 1H) 4.48 (dd, J=11.00, 4.16 Hz, 1H) 7.27 (s, 1H) 7.46 (s, 1H) 7.55 (s, 1H) 7.73-7.84 (m, 8H). Calc'd for C₃₇H₄₃F₆NO₂ (M+H)⁺ 647.32, Found 648.5.

### Example 39

### 2-{5-[1-(3,5-Bis-trifluoromethyl-phenyl)-4-methyl-pentylamino]-4′-trifluoromethyl-biphenyl-3-yl}-4-methyl-pentanoic Acid

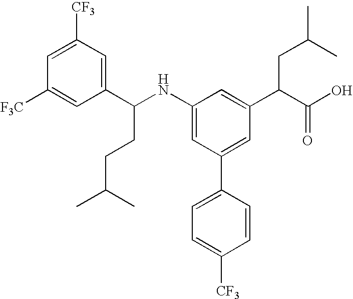

### a) N-Methoxy-N-methyl-3,5-bis-trifluoromethyl-benzamide

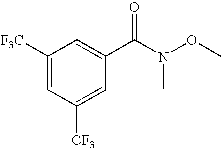

The title compound was prepared via the same procedure as the described in Example 34 step (b) using 3,5-ditrifluoromethylbenzoic acid.

### b) 1-(3,5-Bis-trifluoromethyl-phenyl)-4-methyl-pentan-1-one

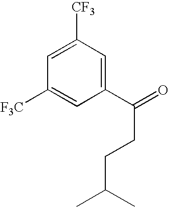

The title compound was prepared via the same procedure as the described in Example 34 step (c) musing compound 39a.

### c) 1-(3,5-Bis-trifluoromethyl-phenyl)-4-methyl-pentylamine

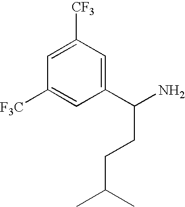

The title compound was prepared via the same procedure as the described in Example 34 step (d) using compound 39b.

MH⁺ 314.3

### d) 2-{5-[1-(3,5-Bis-trifluoromethyl-phenyl)-4-methyl-pentylamino]-4′-trifluoromethyl-biphenyl-3-yl}-4-methyl-pentanoic Acid Ethyl Ester

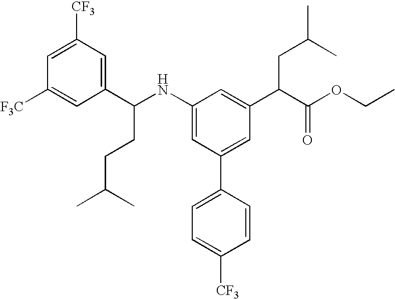

A solution of compound 39c, (0.2 g, 0.64 mmol), triflate from Example 5, step (a) (0.11 g, 0.21 mmol), Pd₂(dba)₃ (0.03 g), dppbiphenyl (0.0 μg), potassium phosphate tribasic (0.11 g, 0.5 mmol), DME (3 mL) in a microwave reaction tube was subjected to microwave irradiation (100° C., 20 min). The solvent was removed by vacuum to give the title compound, 0.08 g crude product. (55%)

MH⁺ 676.3

### e) 2-{5-[1-(3,5-Bis-trifluoromethyl-phenyl)-4-methyl-pentylamino]-4′-trifluoromethyl-biphenyl-3-yl}-4-methyl-pentanoic Acid

A solution of compound 39d (0.08 g), 1N NaOH (1 mL) in THF/MeOH (10 mL/10 mL) was stirred at room temperature for two days. EtOAc (50 mL) added. The organic layer was washed by citric acid aq., NaCl aq., dried with Mg₂SO₄ and evaporated. The crude product was purified by prep TLC (40% EtOAc/Heptane) to give the title compound, 0.012 g (16%)

MH⁺ 648.3

¹H NMR (300 MHz, CD₃OD): δ0.66 (m, 3H), δ0.74 (m, 3H), δ0.81 (m, 6H), δ1.2-1.8 (m, 8H), δ3.4 (m, 1H), δ4.5 (m, 1H), δ6.4 (m, 1H), δ6.7 (m, 2H), δ7.4 (s, 4H), δ7.7 (s, 1H), δ7.9 (s, 2H).

### Example 40

### 2-{5-[(3,5-Difluoro-benzyl)-(3-methyl-butyl)-amino]-4′-trifluoromethyl-biphenyl-3-yl}-4-methyl-pentanoic Acid

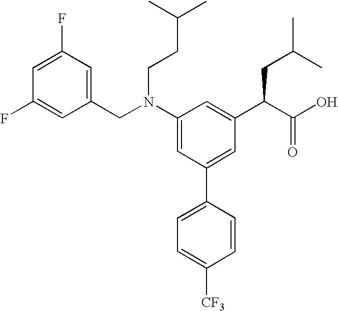

### a) 2-(5-Amino-4′-trifluoromethyl-biphenyl-3-yl)-4-methyl-pentanoic Acid Methyl Ester

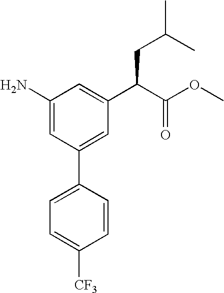

A solution of benzylphenone imine (2.9 mL, 16 mmol), triflate 5a (4 g, 8 mmol), Pd₂(dba)₃ (0.73 g), dppbiphenyl (0.25 g), potassium phosphate tribasic (2 g, 9.4 mmol), DME (20 mL) was placed in equal parts into four microwave reaction tubes. The reaction mixtures were heated in a microwave reactor (100° C., 20 min). LC/MS of the reaction mixture indicated a mixture of 1:1 of the aniline product and imine intermediate. The solid was removed and the filtrate was concentrated. The crude product was dissolved in MeOH and treated with NaOAc (2 g) and NH₂OH—HCl (1 g), and the resulting mixture was stirred at room temperature for 20 min. The imine intermediate was converted to the product as expected. The solvent was removed and the residue was re-dissolved in EtOAc and the solution was washed with water and dried over Na₂SO₄. The crude product was purified by column chromatography (0-30% EtOAc/Heptane) to give the title compound, 2.5 g brown oil. (42% yield), MH⁺ 366.1

### b) 2-[5-(3,5-Difluoro-benzylamino)-4′-trifluoromethyl-biphenyl-3-yl]-4-methyl-pentanoic Acid Methyl Ester

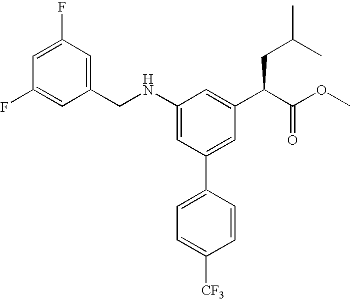

A solution of 3,5-difluor-benzaldehyde (0.09 g, 0.6 mmol), compound 40a (0.23 g, 0.6 mmol) in MeOH (10 mL) was stirred at room temperature for one hour and then NaBH₄ (0.05 g, 13 mmol) was added. The solution was stirred another 30 min. After removing of the solvent, the crude oil was purified by column chromatography (0-30% EtOAc/heptane) to give the title compound, 0.15 g as a colorless oil. (50%). MH⁺ 492.1

### c) 2-{5-[(3,5-Difluoro-benzyl)-(3-methyl-butyl)-amino]-4′-trifluoromethyl-biphenyl-3-yl}-4-methyl-pentanoic Acid Methyl Ester

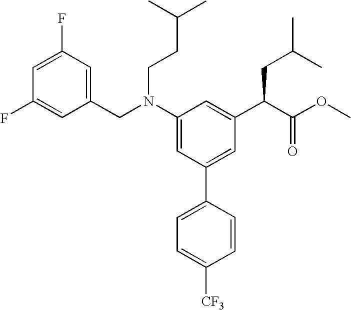

A solution of compound 40b (0.08 g, 0.16 mmol), isovaleraldehyde (0.17 mL, 1.6 mmol) and AcOH (one drop) in DCM (10 mL) was stirred at room temperature overnight. NaBH(OAc)₃ (0.07 g, 0.33 mmol) added and the solution was stirred another 30 min. The solution was treated with EtOAc (50 mL), washed with NaOH (1N) solution and saturated aq. NaCl. The organic layer was dried over Mg₂SO₄ and evaporated. The crude product was purified by column chromatography to give the title compound, 0.07 (76%) MH⁺ 562.3

### d) 2-{5-[(3,5-Difluoro-benzyl)-(3-methyl-butyl)-amino]-4′-trifluoromethyl-biphenyl-3-yl}-4-methyl-pentanoic Acid

A solution of compound 40e (0.07 g) in MeOH/THF/NaOH (1N) (5 mL/5 mL/0.5 mL) was stirred overnight. The reaction solution was concentrated and the residue was purified by Gilson reverse phase HPLC purification. The obtained TFA salt was then converted to the Na salt to yield the title compound, 40 mg (56%).

MH⁺ 548.4

¹H NMR (300 MHz, CD₃OD): δ0.85 (dd, J=6.45 Hz, J=1.12 Hz, 6H), 60.99 (d, 6H, J=6.3 Hz), δ1.5-1.8 (m, 6H), δ3.55 (m, 3H), δ4.6 (s, 2H), δ6.75 (m, 3H), δ6.88 (d, 2H, J=6.5 Hz), δ7.0 (s, 1H), δ7.5 (m, 4H).

While the foregoing specification teaches the principles of the present invention, with examples provided for the purpose of illustration, it will be understood that the practice of the invention encompasses all of the usual variations, adaptations and/or modifications as come within the scope of the following claims and their equivalents. All publications disclosed in the above specification are hereby incorporated by reference in full.

We claim:

1. A method of using compounds of Formula I 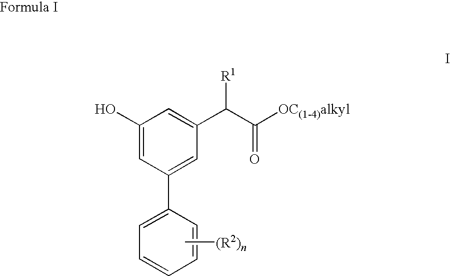
wherein:
R¹ is C₍₁₋₄₎alkyl, or C₍₁₋₅₎alkenyl;
R² is selected from the group consisting of —H, —C₍₁₋₄₎alkyl, —OC₍₁₋₄₎alkyl, —NO₂, —CN, —NH₂, —F, —Br, —Cl, and —CF₃; and
n is an integer from 1-3
to make γ-secretase modulators of Formulas XIII-XXVII 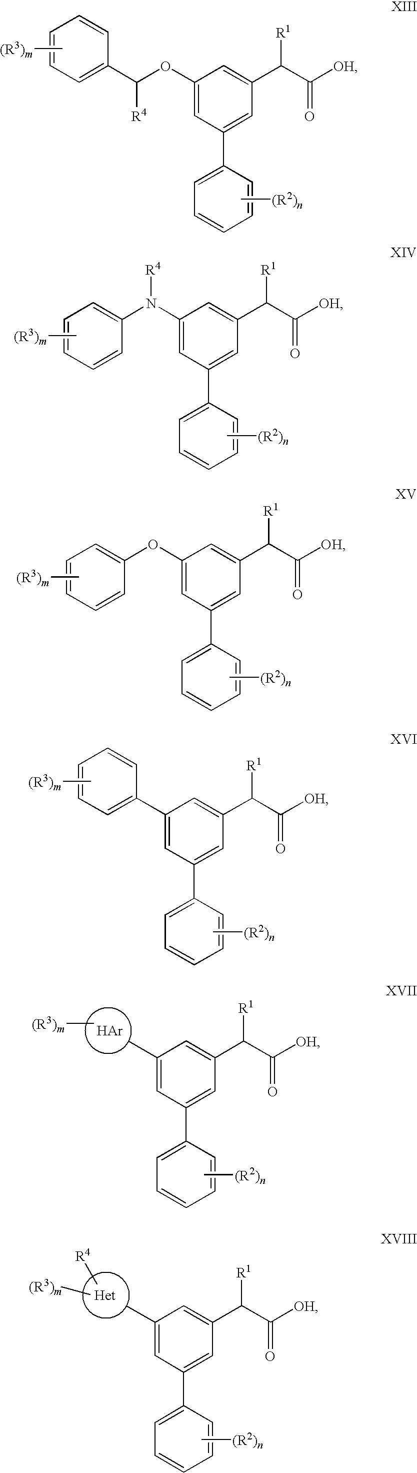 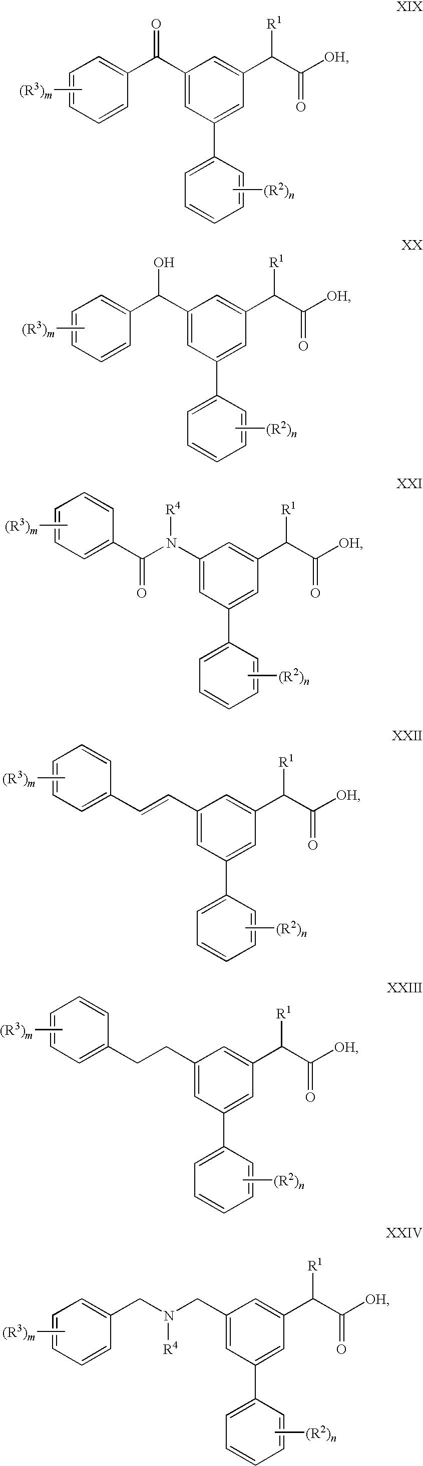 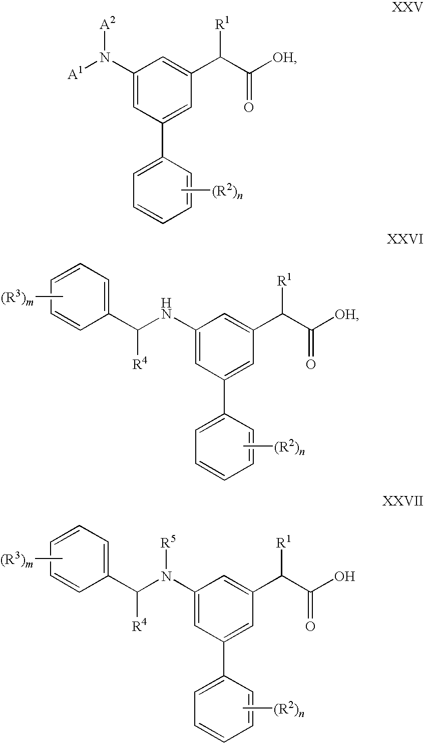
wherein
Het is heterocyclyl;
HAr is heteroaryl;
R³ is selected from the group consisting of —H, —C₍₁₋₄₎alkyl, —OC₍₁₋₄₎alkyl, —NO₂, —CN, —NH₂, —F, —Br, —Cl, and —CF₃;
R⁴ is C₍₁₋₄₎alkyl;
R⁵ is C₍₁₋₄₎alkyl;
A¹ is H or —C₍₁₋₄₎alkyl;
A² is —C₍₁₋₄₎alkyl;
alternatively, A¹ and A² may be taken together to form a nitrogen containing heterocyclic ring selected from the following: 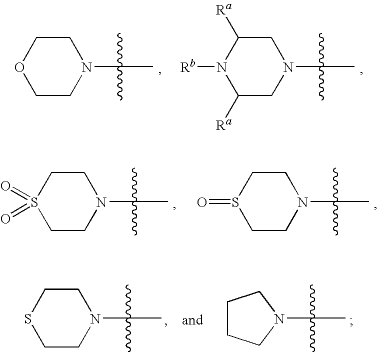
wherein:
R^{a} is H, CH₃, or CH₂CH₃; and
R^{b} is H, or CH₃;
m is an integer from 1-3;
n is an integer from 1-3; and
solvates, hydrates, prodrugs, and pharmaceutically acceptable salts thereof.

2. The method of claim 1, wherein said γ-secretase modulators are of Formula XIII 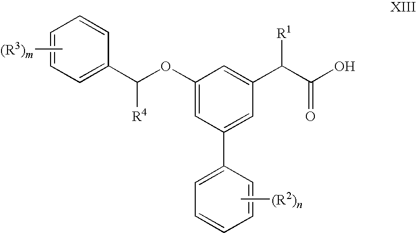
and solvates, hydrates, prodrugs, and pharmaceutically acceptable salts thereof.

3. The method of claim 1, wherein said γ-secretase modulators are of Formula XIV 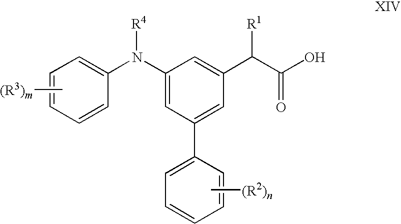
and solvates, hydrates, prodrugs, and pharmaceutically acceptable salts thereof.

4. The method of claim 1, wherein said γ-secretase modulators are of Formula XV 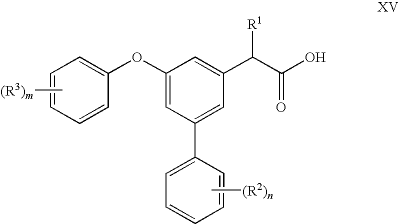
and solvates, hydrates, prodrugs, and pharmaceutically acceptable salts thereof.

5. The method of claim 1, wherein said γ-secretase modulators are of Formula XVI 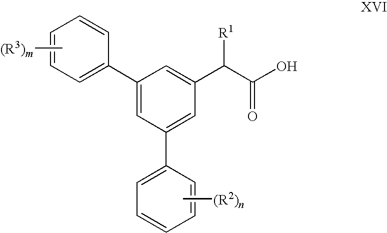
and solvates, hydrates, prodrugs, and pharmaceutically acceptable salts thereof.

6. The method of claim 1, wherein said γ-secretase modulators are of Formula XVII 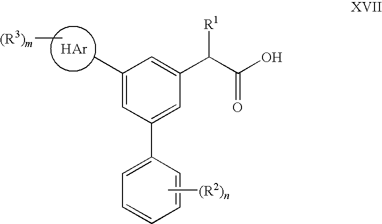
and solvates, hydrates, prodrugs, and pharmaceutically acceptable salts thereof.

7. The method of claim 1, wherein said γ-secretase modulators are of Formula XVIII 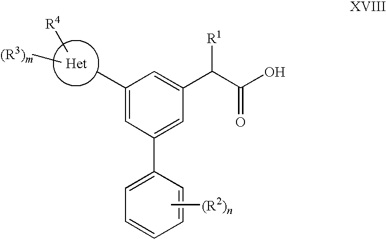
and solvates, hydrates, prodrugs, and pharmaceutically acceptable salts thereof.

8. The method of claim 1, wherein said γ-secretase modulators are of Formula XIX 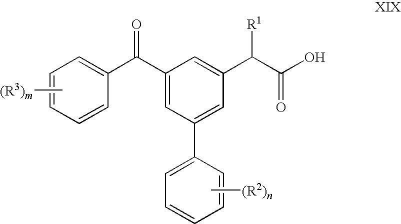
and solvates, hydrates, prodrugs, and pharmaceutically acceptable salts thereof.

9. The method of claim 1, wherein said γ-secretase modulators are of Formula XX 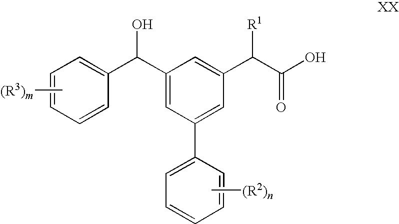
and solvates, hydrates, prodrugs, and pharmaceutically acceptable salts thereof.

10. The method of claim 1, wherein said γ-secretase modulators are of Formula XXI 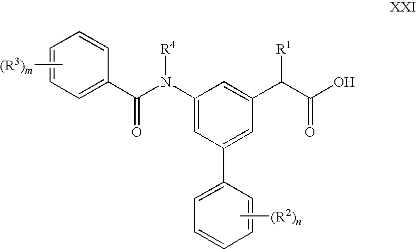
and solvates, hydrates, prodrugs, and pharmaceutically acceptable salts thereof.

11. The method of claim 1, wherein said γ-secretase modulators are of Formula XXII 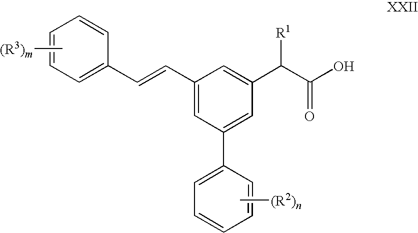
and solvates, hydrates, prodrugs, and pharmaceutically acceptable salts thereof.

12. The method of claim 1, wherein said γ-secretase modulators are of Formula XXIII 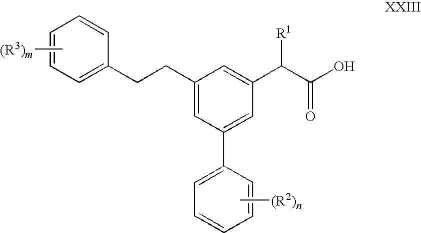
and solvates, hydrates, prodrugs, and pharmaceutically acceptable salts thereof.

13. The method of claim 1, wherein said γ-secretase modulators are of Formula XXIV 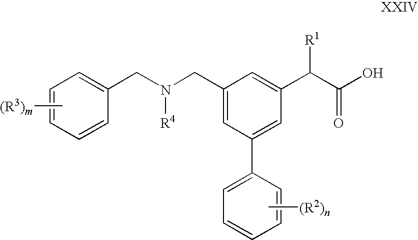
and solvates, hydrates, prodrugs, and pharmaceutically acceptable salts thereof.

14. The method of claim 1, wherein said γ-secretase modulators are of Formula XXV 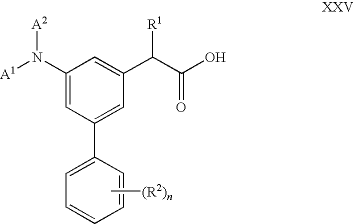
and solvates, hydrates, prodrugs, and pharmaceutically acceptable salts thereof.

15. The method of claim 1, wherein said γ-secretase modulators are of Formula XXVI 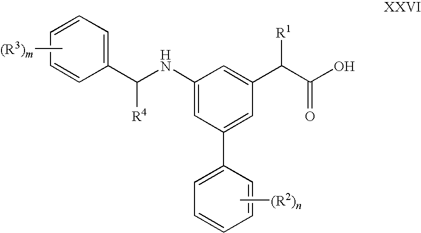
and solvates, hydrates, prodrugs, and pharmaceutically acceptable salts thereof.

16. The method of claim 1, wherein said γ-secretase modulators are of Formula XXVII 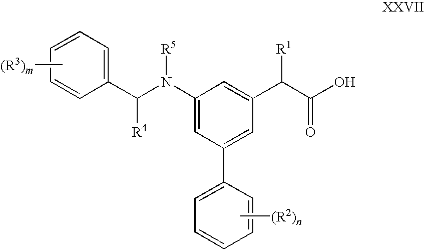
and solvates, hydrates, prodrugs, and pharmaceutically acceptable salts thereof.

17. A method of using compounds of Formula I 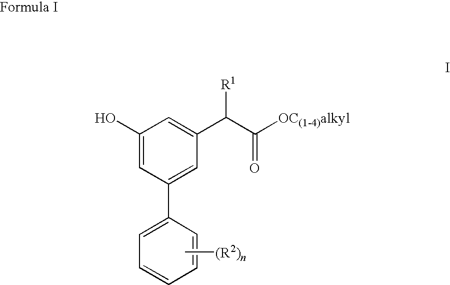
wherein:
R¹ is C₍₁₋₄₎alkyl;
R² is selected from the group consisting of —F, —Br, —Cl, and —CF₃; and
n is an integer from 1-3
to make γ-secretase modulators of Formulas XIII-XXVII 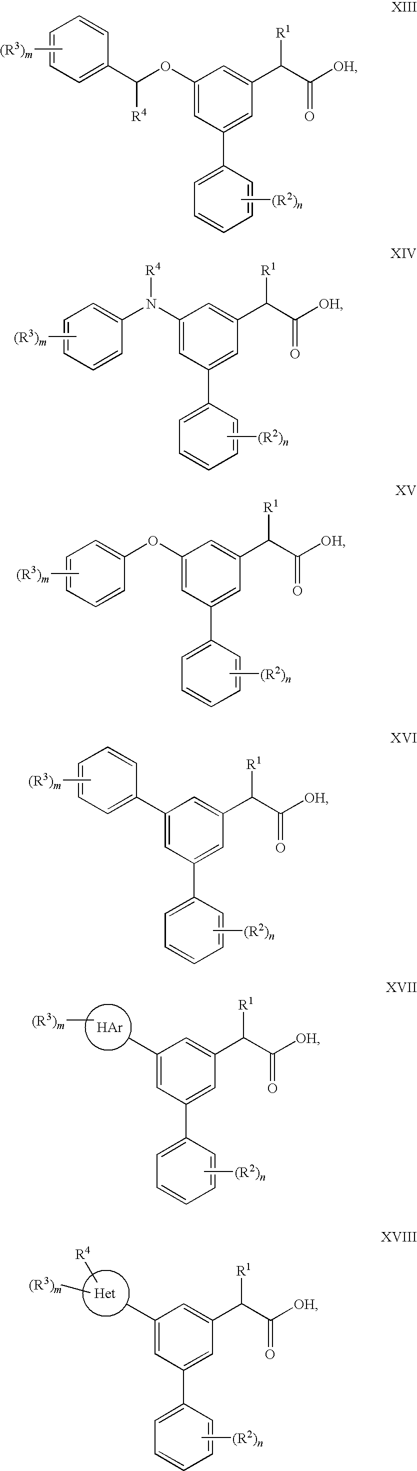 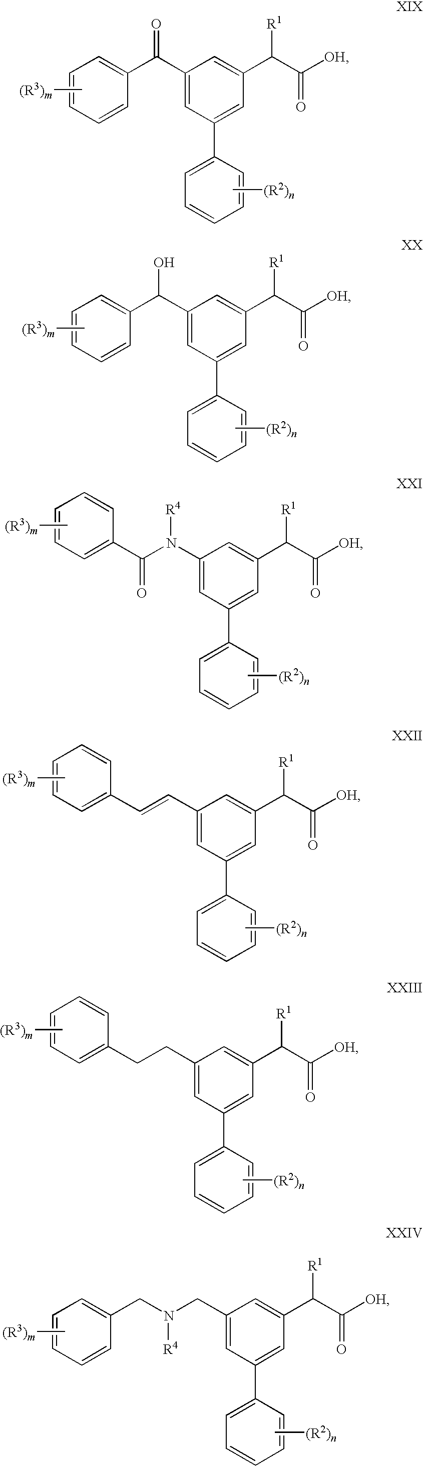 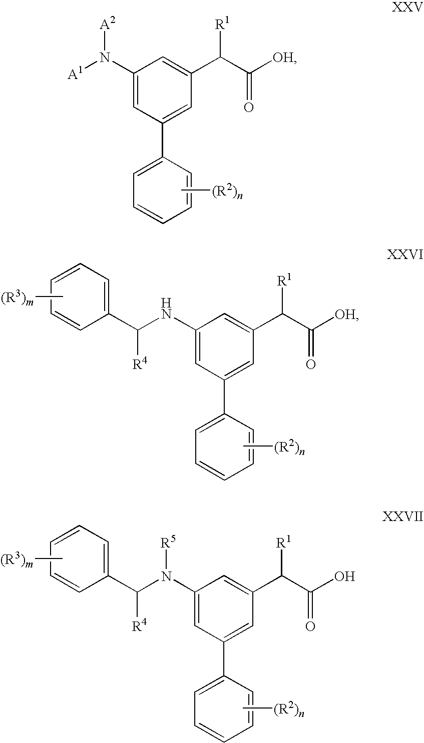
wherein
Het is heterocyclyl;
HAr is heteroaryl;
R³ is selected from the group consisting of —F, —Br, —Cl, and —CF₃;
R⁴ is C₍₁₋₄₎alkyl;
R⁵ is C₍₁₋₄₎alkyl;
A¹ is H or —C₍₁₋₄₎alkyl;
A² is —C₍₁₋₄₎alkyl;
alternatively, A¹ and A² may be taken together to form a nitrogen containing heterocyclic ring selected from the following: 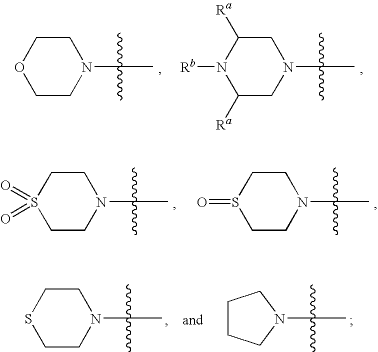
wherein:
R^{a} is H, CH₃, or CH₂CH₃; and
R^{b} is H, or CH₃;
m is an integer from 1-3;
n is an integer from 1-3; and
solvates, hydrates, prodrugs, and pharmaceutically acceptable salts thereof.

18. A method of selective mono-debenzylation of a 3,5 bis-benzyloxy moiety, such as: 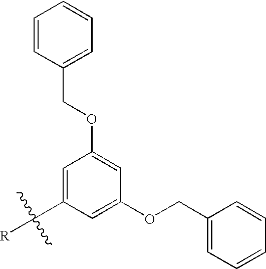
characterized by the use of 1 to 1.1 equivalents of a base selected from the group consisting of NaH, KH, NaOH, KOH, LiOH, KOtBu, NaOtBu, K₂CO₃, Na₂CO₃, Cs₂CO₃ and NaN(Si(CH₃)₃)₂, or LiN(Si(CH₃)₃)₂ and one equivalent of hydrogen.
